(19) United States
(12) Patent Application Publication
(10) Pub. No.: US 2026/0193289 A1
(43) Pub. Date: Jul. 9, 2026

(54) PROCESSABLE STATINS AND USE FOR THE SAME

(71) Applicant: Ripple Therapeutics Corporation, Toronto (CA)

(72) Inventors: Matthew Alexander John STATHAM, Milton (CA); Jonathan DAY, Toronto (CA); Gregory Allan WHITTON, Toronto (CA); Kyle Giovanni BATTISTON, Toronto (CA); Ian Charles PARRAG, Mississauga (CA); Madeline Jenna SIMPSON, Toronto (CA); Wendy Alison NAIMARK, Toronto (CA)

(73) Assignee: Ripple Therapeutics Corporation, Toronto (CA)

(21) Appl. No.: 19/424,652

(22) Filed: Dec. 18, 2025

Related U.S. Application Data
(60) Provisional application No. 63/736,324, filed on Dec. 19, 2024.
(60) Provisional application No. 63/736,331, filed on Dec. 19, 2024.
(60) Provisional application No. 63/797,800, filed on Apr. 30, 2025.
(60) Provisional application No. 63/797,807, filed on Apr. 30, 2025.

(51) Int. Cl. C07J 43/00; A61K 9/00; A61K 31/58; A61P 27/02

(52) U.S. Cl. CPC C07J 43/003; A61K 9/0051; A61K 31/58; A61P 27/02

(57) ABSTRACT

Described herein are formulations for the controlled release of statin therapies and methods related thereto.

### CROSS-REFERENCE

This application claims the benefit of U.S. Provisional Application No. 63/736,324, filed Dec. 19, 2024, U.S. Provisional Application No. 63/736,331, filed Dec. 19, 2024, U.S. Provisional Application No. 63/797,800, filed Apr. 30, 2025, and U.S. Provisional Application No. 63/797,807, filed Apr. 30, 2025, each of which are incorporated herein by reference in their entirety.

### BACKGROUND OF THE INVENTION

The global controlled release drug delivery market size in 2022 was estimated at over $50B. While there are some controlled delivery systems for delivering an active pharmaceutical ingredient (API) to a treatment site, the systems generally contain polymer carriers that can create technical and/or regulatory challenges, such as, for example, burst release kinetics, low drug loading, and adverse inflammatory responses. Providing a therapeutic consisting entirely or almost entirely of API could address such technical and regulatory challenges.

### SUMMARY OF THE INVENTION

Provided in some embodiments herein are compounds comprising a first radical (D1) and a second radical (D2) (e.g., having the formula: D1-L-D2). In certain instances, D1 is a processable group (also referred to herein as a processable radical), L is a linker, and D2 is a drug (also referred herein as a drug radical). In some embodiments, L is a hydrolyzable linker, such that when the compound of formula D1-L-D2 is (e.g., intraocularly) administered (or when present in or otherwise exposed to an aqueous environment, such as a buffering solution, tears, serum, or the like), D1 and D2 are released (e.g., in their free, non-radical form). In certain instances, a drug (such as a therapeutically active agent (e.g., a statin or an active form thereof) provided herein) is joined with a processable group (such as a steroid or other radical of a formula described herein, such as Formula (V) or Formula (V′)). In certain instances, a drug (such as a statin (or an active form thereof) provided herein) is joined with a processable group (such as a steroid or other radical of a formula described herein, such as Formula (V) or Formula (V′)). In certain instances, the processable group D1 may or may not itself be processable when in free form, but when combined with D2 (e.g., through a linker L) provides a solid (e.g., at a physiological temperature) that is processable (e.g., at a temperature above a physiological temperature).

Provided in certain instances herein is a platform for providing compounds and implants (e.g., with high drug content, low excipient content (e.g., that would otherwise need to be removed), and other benefits, such as described herein) that provide long-lasting release of therapeutics (e.g., a therapeutically active agent (e.g., statin, steroid, or active forms thereof) provided herein) in biological and therapeutic applications, such as in ocular (e.g., implant) administration and/or via implantation or insertion. Provided in certain instances herein is a platform for providing compounds and implants (e.g., with high drug content, low excipient content (e.g., that would otherwise need to be removed), and other benefits, such as described herein) that provide long-lasting release of therapeutics (e.g., statins, steroids, and/or active forms thereof) in biological and therapeutic applications such as via implantation and/or in ocular (e.g., implant) administration or insertion.

In some instances, compounds provided herein are processable into forms (e.g., implants or other bodies), such as forms suitable for being administered to or inserted into (e.g., an eye of) an individual. In some instances, such compounds are processable without the need for additional excipients or materials (e.g., controlled release polymers, matrices, or other components). In certain instances, no or low amounts of additional excipients or materials facilitate high levels of drug delivery, while limiting impact of drug delivery (e.g., a small implant can have high quantities of drug).

In certain instances, such compounds (or implants comprising such compounds) are administered to (e.g., implanted or inserted into) an individual, such that sustained and/or otherwise controlled (e.g., local) delivery of the drug is achieved. In some instances, delivery of the compounds (e.g., in the form of an implant, etc.) facilitate delivery of a drug component for an extended period of time, such as for weeks, months, or more. In certain instances, compounds, formulations, and implants provided herein facilitate the longterm delivery of drugs to an individual, such as without the need for frequent dosing.

In some embodiments, the group D2 of a compound described herein is an active agent or drug. In some embodiments, the group D1 of a compound described herein is also an active agent or drug. In some embodiments, D1 and D2 of a compound described herein are both effective in the treatment of a single indication, such that administration of a compound herein provides a combination therapeutic effect. In some instances, D1 provides a therapeutic benefit (e.g., in its free form) and D2 treats or prevents an effect (e.g., a side-effect) of D1 (in its free form). In some embodiments, D1 is an inactive or benign agent (e.g., an agent that (in its free form) does not provide a (e.g., significant, measurable, and/or direct) therapeutic effect and/or benefit (e.g., to an individual (e.g., in need thereof)) and D2 is an active agent (e.g., a therapeutically active agent) (or drug). In some embodiments, the therapeutically active agent is released (e.g., at a controlled rate (e.g., over an extended period of time)) from a composition provided herein (e.g., an article or implant). In some embodiments, such as wherein the compound is formulated as or with an implant, the inactive agent acts as a carrier for providing a therapeutically active agent to a (e.g., treatment site of) an individual (e.g., in need thereof) (e.g., for an extended period of time) and the therapeutic agent provides a therapeutically beneficial effect for an extended period of time.

Provided in some embodiments herein are compounds, such as described herein, (e.g., pharmaceutical) compositions comprising compounds described herein, and methods of making and using compounds provided herein. In some embodiments, methods of using the compounds provided herein include methods of treating disorders in individuals in need thereof, such as disorders treatable by a drug D2 (e.g., in its free form). In some embodiments, methods of treatment provided herein comprise methods of treating ocular disorders, such as (e.g., dry) age-related macular degeneration (AMD). It is to be understood that disclosures of methods provided herein explicitly include disclosures of pharmaceutical compositions comprising (e.g., an effective amount) of a compound provided herein for such uses.

In some instances, provided herein is a compound that delivers a therapeutically effective amount of (e.g., a free form of) a therapeutic agent (e.g., steroid and/or a statin), such as a therapeutically active agent (e.g., steroid, statin, or active forms thereof) described herein.

Provided in some embodiments herein is a compound having a structure represented by Formula I:

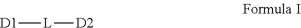

or a pharmaceutically acceptable salt thereof,
wherein:
   D1 is a radical of a steroid;
   D2 is a radical of a statin; and
   L is a bond.

In some embodiments, D1 has a structure provided in Table 1.

In some embodiments, D1 is a radical of anecortave.

In some embodiments, D2 has a structure provided in Table 2.

In some embodiments, D2 is a radical of a statin selected from the group consisting of atorvastatin (radical), rosuvastatin (radical), cerivastatin (radical), fluvastatin (radical), pitavastatin (radical), an active (e.g., carboxylate) form of lovastatin (radical), an active (e.g., carboxylate) form of mevastatin (radical), an active (e.g., carboxylate) form simvastatin (radical), and pravastatin (radical). In some embodiments, D2 is a radical of a statin selected from the group consisting of cerivastatin (radical), fluvastatin (radical), pitavastatin (radical), an active (e.g., carboxylate) form of lovastatin (radical), an active (e.g., carboxylate) form of mevastatin (radical), an active (e.g., carboxylate) form simvastatin (radical), and pravastatin (radical). In some embodiments, D2 is a radical of atorvastatin, rosuvastatin, cerivastatin, fluvastatin, or pitavastatin. In some embodiments, D2 is a radical of cerivastatin, fluvastatin, or pitavastatin. In some embodiments, D2 is a radical of an active (e.g., carboxylate) form of lovastatin, a radical of an active (e.g., carboxylate) form of mevastatin, a radical of an active (e.g., carboxylate) form of simvastatin, or pravastatin. In some embodiments, D2 is a radical of rosuvastatin.

Provided in some embodiments herein is a compound having a structure represented by Formula IC:

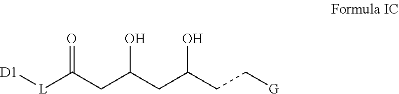

wherein:
   [symbol] is a single bond or a double bond;
   G is substituted heteroaryl or substituted carbocyclyl;
   D1 is a radical of a steroid; and
   L is a linker (e.g., a bond),
   or a pharmaceutically acceptable salt or solvate thereof.

In some embodiments, the hydroxyl groups of the structure represented by Formula IC are meso.

In some embodiments, D1 is a radical of a steroid selected from the group consisting of anecortave (radical), dexamethasone (radical), hydrocortisone (radical), triamcinolone (radical), or prednisolone (radical).

In some embodiments, D1 has a structure provided in Table 1 (e.g., anecortave desacetate).

In some embodiments, D1 is a radical of a steroid selected from the group consisting of anecortave (radical), dexamethasone (radical), hydrocortisone (radical), triamcinolone (radical), or prednisolone (radical). In some embodiments, D1 is a radical of anecortave, such as a radical of anecortave described herein.

In some embodiments, D1 is a radical of anecortave desacetate.

In some embodiments, D1 has a structure represented by Formula C1:

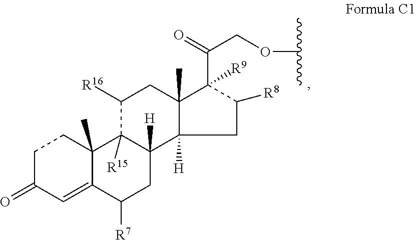

wherein:
   [symbol] is a single bond or a double bond;
   R⁷ is hydrogen or halogen;
   R⁸ is hydrogen or C₁-C₄ alkyl;
   R⁹ is absent, hydrogen, or hydroxyl;
   R¹⁵ is absent, hydrogen, or halogen; and
   R¹⁶ is hydrogen or hydroxyl,
   or a pharmaceutically acceptable salt or solvate thereof.

In some embodiments, R⁷ is hydrogen.

In some embodiments, R⁸ is hydrogen.

In some embodiments, R⁹ is hydroxyl.

In some embodiments, R¹⁵ is absent.

In some embodiments, R¹⁶ is hydrogen.

In some embodiments, D1 has a structure represented by Formula C2:

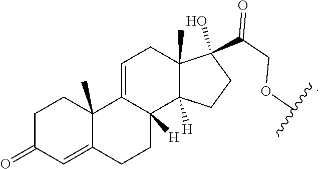 [SMILES: *OCC(=O)[C@@]1(O)CC[C@H]2[C@@H]3CCC4=CC(=O)CC[C@]4(C)C3=CC[C@@]21C]

In some embodiments, D1 is a radical of anecortave.

In some embodiments, G is substituted heteroaryl. In some embodiments, the substituted heteroaryl is selected from the group consisting of substituted pyrrolyl, substituted pyridinyl, substituted indolyl, substituted quinolinyl, and substituted pyrimidinyl.

In some embodiments, G is heteroaryl substituted with one or more substituents, each substituent being independently selected from the group consisting of substituted or unsubstituted amide, substituted or unsubstituted alkyl, substituted or unsubstituted heteroalkyl, substituted or unsubstituted aryl, substituted or unsubstituted carbocyclyl, and substituted or unsubstituted sulfonamide.

In some embodiments, G is heteroaryl substituted with one or more substituents, each substituent being independently selected from the group consisting of substituted or unsubstituted alkyl, substituted or unsubstituted heteroalkyl, substituted or unsubstituted aryl, substituted or unsubstituted carbocyclyl, and substituted or unsubstituted sulfonamide.

In some embodiments, G is pyridinyl substituted with branched alkyl (e.g., isopropyl), phenyl substituted with fluoro, and unsubstituted heteroalkyl (e.g., CH₂OCH3).

In some embodiments, G is pyridinyl substituted with branched alkyl (e.g., isopropyl) and phenyl substituted with fluoro.

In some embodiments, G is quinolinyl substituted with cyclopropyl and phenyl substituted with fluoro.

In some embodiments, G is pyrimidinyl substituted with branched alkyl (e.g., isopropyl), substituted aryl (e.g., phenyl substituted with fluoro), and sulfonamide substituted with alkyl (e.g., N-methylmethanesulfonamidyl). In some embodiments, G is pyrimidinyl substituted with branched alkyl (e.g., isopropyl), phenyl substituted with fluoro, and N-methylmethanesulfonamidyl.

Provided in some embodiments herein is a compound having a structure represented by Formula III:

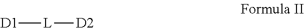

or a pharmaceutically acceptable salt thereof,
wherein:
   D1 is a radical of a steroid;
   D2 is a radical of rosuvastatin; and
   L is a linker (e.g., a bond).

Provided in some embodiments herein is a compound having a structure represented by Formula IIC:

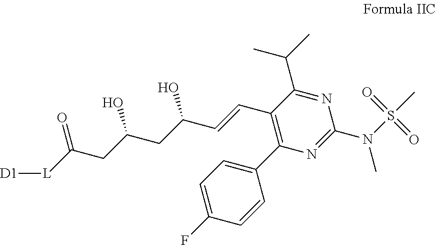

wherein:
   D1 is a radical of a steroid; and
   L is a linker (e.g., a bond),
   or a pharmaceutically acceptable salt or solvate thereof.

In some embodiments, D1 is a radical of anecortave.

In some embodiments, G is substituted carbocyclyl.

In some embodiments, a compound described herein has a structure represented by Formula D:

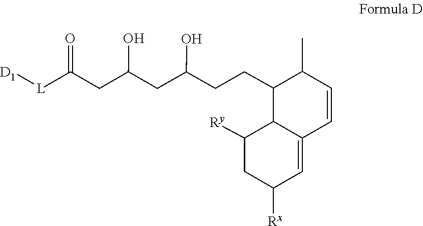

wherein:
   D1 is a radical of a steroid;
   L is a linker (e.g., a bond);
   R^{x} is hydrogen, alkyl, or hydroxyl; and
   R^{y} is substituted alkoxy.

In some embodiments, the hydroxyl groups of the structure represented by Formula D are meso.

In some embodiments, R^{x} is hydrogen. In some embodiments, R^{x} is methyl. In some embodiments, R^{x} is hydroxyl.

In some embodiments, R^{y} is branched alkoxy substituted with oxo. In some embodiments, R^{y} is —O(C═O)CH(CH₃)CH₂CH₃ or —O(C═O)C(CH₃)₂CH₂CH₃.

Provided in some embodiments herein is a compound having a structure represented by Formula IA:

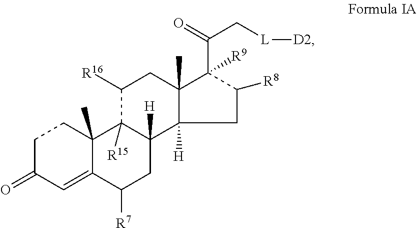

wherein:
   [symbol] is a single bond or a double bond;
   R⁷ is hydrogen or halogen;
   R⁸ is hydrogen or C₁-C₄ alkyl;
   R⁹ is absent, hydrogen, or hydroxyl;
   R¹⁵ is absent, hydrogen, or halogen;
   R¹⁶ is hydrogen or hydroxyl;
   D2 is a radical of a statin; and
   L is a linker (e.g., a bond),
   or a pharmaceutically acceptable salt or solvate thereof.

Provided in some embodiments herein is a compound having a structure represented by Formula IIA:

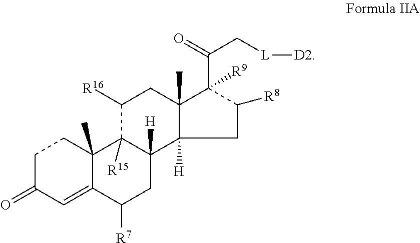

wherein:
   [symbol] is a single bond or a double bond;
   R⁷ is hydrogen or halogen;
   R⁸ is hydrogen or C₁-C₄ alkyl;
   R⁹ is absent, hydrogen, or hydroxyl;
   R¹⁵ is absent, hydrogen, or halogen;
   R¹⁶ is hydrogen or hydroxyl;
   D2 is a radical of rosuvastatin; and
   L is a linker (e.g., a bond),
   or a pharmaceutically acceptable salt or solvate thereof.

In some embodiments, R⁷ is hydrogen.

In some embodiments, R⁸ is hydrogen.

In some embodiments, R⁹ is hydroxyl.

In some embodiments, R¹⁵ is absent.

In some embodiments, R¹⁶ is hydrogen.

In some embodiments, a compound described herein has a structure represented by Formula IB:

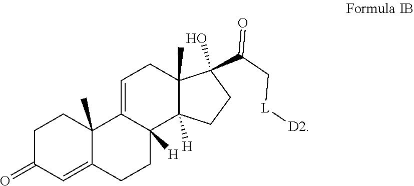

Unless stated otherwise, Formula IB and Formula IIB are referred to interchangeably herein.

In some embodiments, D2 has a structure represented by Formula B1:

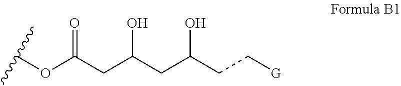

wherein:
   [symbol] is a single bond or a double bond; and
   G is substituted heteroaryl or substituted carbocyclyl.

In some embodiments, the hydroxyl groups of the structure represented by Formula B1 are meso.

In some embodiments, G is substituted carbocyclyl.

In some embodiments, D2 has a structure represented by Formula B2:

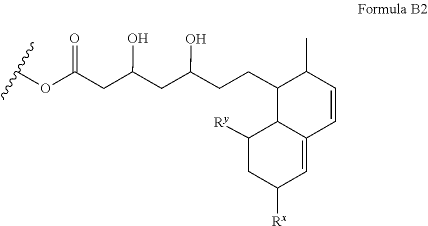

wherein:
   R^{x} is hydrogen, alkyl, or hydroxyl; and
   R^{y} is substituted alkoxy.

In some embodiments, the hydroxyl groups of the structure represented by Formula B2 are meso.

In some embodiments, R^{x} is hydrogen. In some embodiments, R^{x} is methyl. In some embodiments, R^{x} is hydroxyl.

In some embodiments, R^{y} is branched alkoxy substituted with oxo. In some embodiments, R^{y} is —O(C═O)CH(CH₃)CH₂CH₃ or —O(C═O)C(CH₃)₂CH₂CH₃.

In some embodiments, G is substituted heteroaryl. In some embodiments, the substituted heteroaryl is selected from the group consisting of substituted pyrrolyl, substituted pyridinyl, substituted indolyl, substituted quinolinyl, and substituted pyrimidinyl.

In some embodiments, G is heteroaryl substituted with one or more substituents, each substituent being independently selected from the group consisting of substituted or unsubstituted amide, substituted or unsubstituted alkyl, substituted or unsubstituted heteroalkyl, substituted or unsubstituted aryl, substituted or unsubstituted carbocyclyl, and substituted or unsubstituted sulfonamide.

In some embodiments, G is heteroaryl substituted with one or more substituents, each substituent being independently selected from the group consisting of substituted or unsubstituted alkyl, substituted or unsubstituted heteroalkyl, substituted or unsubstituted aryl, substituted or unsubstituted carbocyclyl, and substituted or unsubstituted sulfonamide.

In some embodiments, G is pyridinyl substituted with branched alkyl (e.g., isopropyl), phenyl substituted with fluoro, and unsubstituted heteroalkyl (e.g., CH₂OCH3).

In some embodiments, G is pyridinyl substituted with branched alkyl (e.g., isopropyl) and phenyl substituted with fluoro.

In some embodiments, G is quinolinyl substituted with cyclopropyl and phenyl substituted with fluoro.

In some embodiments, G is pyrimidinyl substituted with branched alkyl (e.g., isopropyl), substituted aryl (e.g., phenyl substituted with fluoro), and sulfonamide substituted with alkyl (e.g., N-methylmethanesulfonamidyl).

In some embodiments, G is pyrimidinyl substituted with branched alkyl (e.g., isopropyl), phenyl substituted with fluoro, and N-methylmethanesulfonamidyl.

In some embodiments, D2 has a structure represented by Formula A:

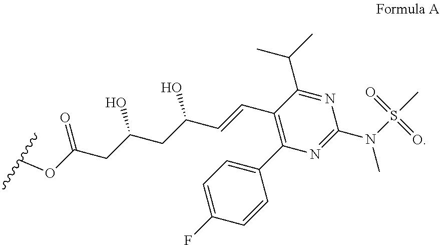 [SMILES: *OC(=O)C[C@H](O)C[C@H](O)/C=C/c1c(-c2ccc(F)cc2)nc(N(C)S(C)(=O)=O)nc1C(C)C]

In some embodiments, the linker (e.g., L) is or comprises (e.g., a diradical (e.g., a molecular species (e.g., an organic compound) with two electrons occupying degenerate molecular orbitals) of) one or more linker groups, each linker group being independently selected from any linker or linker group provided herein (e.g., any linker or linker group provided in Table 3).

In some embodiments, L is a bond, substituted or unsubstituted alkyl, substituted or unsubstituted heteroalkyl, substituted or unsubstituted alkoxy, substituted or unsubstituted carbocyclyl, or substituted or unsubstituted heterocyclyl.

In some embodiments, the linker (e.g., L) is a bond.

In some embodiments, L is a bond.

In some embodiments, the linker (e.g., L) is or comprises substituted or unsubstituted alkyl. In some embodiments, the linker (e.g., L) is substituted alkyl. In some embodiments, the linker (e.g., L) is or comprises alkyl substituted with oxo. In some embodiments, the linker (e.g., L) is or comprises —(O═C)CH₂—.

In some embodiments, the linker (e.g., L) is or comprises:

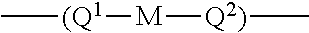

wherein:
   Q¹ and Q² are each independently absent or (C═X¹)X²;
      X¹ is O or S;
      X² is O, S, or NR¹;
         R¹ is hydrogen or C₁-C₆ alkyl; and
   M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl).

In some embodiments, Q¹ and Q² are each independently absent, C═O, C═S, (C═O)O, (C═O)S, (C═O)N, or (C═S)S. In some embodiments, Q¹ and Q² are each independently absent, C═O, or (C═O)O.

In some embodiments, M is substituted or unsubstituted alkyl (e.g., C₁-C₆ alkyl), substituted or unsubstituted heteroalkyl (e.g., C₁-C₆ heteroalkyl), or substituted or unsubstituted aryl.

In some embodiments, M is substituted or unsubstituted alkyl (e.g., C₁-C₆ alkyl (e.g., alkyl-carbocyclyl-alkyl)) or substituted or unsubstituted heteroalkyl (e.g., C₁-C₆ heteroalkyl).

In some embodiments, a compound described herein has a structure of a compound in Table 4.

Provided in some embodiments herein is a pharmaceutical implant or article comprising a compound described herein, or a pharmaceutically-acceptable salt thereof.

In some embodiments, the implant or article comprises at least 50 wt. % of the compound or pharmaceutically acceptable salt thereof. In some embodiments, the implant or article comprises at least 70 wt. % of the compound or pharmaceutically acceptable salt thereof.

In some embodiments, the implant or article comprises at least 90 wt. % of the compound or pharmaceutically acceptable salt thereof.

In some embodiments, the implant or article undergoes surface erosion to release the compound, D1 (in its free form), and/or D2 (in its free form).

In some embodiments, D1 (e.g., a steroid radical) and D2 (e.g., a statin radical, such as a rosuvastatin radical described herein) are released (in their free form) from the pharmaceutical implant or article at near zero-order in a buffered solution or in vivo.

In some embodiments, D1 (e.g., a steroid radical) and D2 (e.g., a statin radical, such as a rosuvastatin radical described herein) are released from the pharmaceutical implant or article (in their free form) at 37° C. in 100% bovine serum or at 37° C. in phosphate buffered saline (PBS) at a rate such that t₁₀ is greater than or equal to 1/10 of t₅₀.

In some embodiments, D1 (e.g., a steroid radical) and D2 (e.g., a statin radical) are released from the pharmaceutical implant or article (in their free form) at 37° C. in 1% fetal bovine serum (FBS) in phosphate buffered saline (PBS) at a rate such that t₁₀ is greater than or equal to 1/10 of t₅₀.

In some embodiments, the implant or article is in a form suitable for an administration route described herein. In some embodiments, the implant or article is in a form suitable for ophthalmic administration (e.g., intraocular insertion). In some embodiments, the implant or article is in a form suitable for intravitreal administration. In some embodiments, the implant or article is a shaped article. In some embodiments, the implant or article is cylindrical.

In some embodiments, an implant or article provided herein is about 0.01 millimeters (mm) to about 50 mm in diameter. In some embodiments, an implant or article provided herein is about 0.1 millimeters (mm) to about 25 mm in diameter. In some embodiments, the implant or article is 0.5 mm to 25 mm in length. In some embodiments, the implant or article is 0.01 to 1 mm in diameter and 0.5 to 15 mm in length. In some embodiments, the implant or article is about 0.2 mm in diameter and about 1 mm in length. In some embodiments, the implant or article is about 0.2 mm in diameter and about 6 mm in length.

In some embodiments, any implant or article provided herein is an implant, such as suitable for intraocular insertion. In some embodiments, the implant is a rod, fiber, pellet, or particle (e.g., microparticle).

In some embodiments, the implant or article is in a form suitable for intraocular insertion.

Provided in some embodiments herein is a method of administering (e.g., inserting) an implant or article described herein to (e.g., an eye of) an individual. In some embodiments, the implant or article is administered to treat an indication described herein.

Provided in some embodiments herein is a method of modulating lipids in an eye of an individual, the method comprising administering to the eye of the individual a compound, pharmaceutically acceptable salt, implant, article, or composition of any embodiments provided herein. In some embodiments, modulating lipids comprises reducing lipoprotein levels, reducing lipoprotein production, reducing drusen levels, reducing drusen formation, reducing cholesterol levels, reducing cholesterol synthesis, reducing lipid peroxidation, or a combination thereof (in the eye of the individual).

Provided in some embodiments herein is a method of reducing inflammation in an eye of an individual, the method comprising administering to the eye of the individual a compound, pharmaceutically acceptable salt, implant, article, or composition of any embodiments provided herein. In some embodiments, reducing inflammation comprises decreasing oxidative stress, lipid peroxidation, and/or pro-inflammatory factors in the eye of the individual.

Provided in some embodiments herein is a method of treating an ophthalmic disease or disorder in an individual in need thereof, the method comprising administering to the individual a compound, pharmaceutically acceptable salt, implant, article, or composition described herein. In some embodiments, the ophthalmic disease or disorder is macular degeneration. In some embodiments, the ophthalmic disease or disorder is age-related macular degeneration (AMD). In some embodiments, the ophthalmic disease or disorder is wet macular degeneration. In some embodiments, the ophthalmic disease or disorder is wet AMD. In some embodiments, the ophthalmic disease or disorder is dry macular degeneration. In some embodiments, the ophthalmic disease or disorder is dry AMD (e.g., intermediate AMD or geographic atrophy). In some embodiments, the ophthalmic disease or disorder is intermediate age-related macular degeneration. In some embodiments, the ophthalmic disease or disorder is geographic atrophy.

In some embodiments, the article or implant described herein is at least partially biodegradable. In some embodiments, the article or implant is non-biodegradable.

In some embodiments, removal of the article or implant is not required (e.g., because the implant is completely or almost completely (e.g., bio- or physiologically) degraded or degradable (e.g., at least 80 wt. %, at least 85 wt. %, at least 90 wt. %, at least 95 wt. %, at least 98 wt. %, at least 99 wt. %, or the like)). In some embodiments, the article or implant is not removed (e.g., because the implant is completely or almost completely (e.g., bio- or physiologically) degraded or degradable (e.g., at least 80 wt. %, at least 85 wt. %, at least 90 wt. %, at least 95 wt. %, at least 98 wt. %, at least 99 wt. %, or the like)).

In some embodiments, any article or implant provided herein releases (e.g., by surface erosion) the compound or a free form of a compound released therefrom, such as for one week or more. In some embodiments, the article or implant releases (e.g., by surface erosion) the compound or a free form of a compound released therefrom one month or more (e.g., two or more months, three or more months, or four or more months). In some embodiments, the article or implant is tolerated in the (e.g., eye of the) individual for one month or more.

In some embodiments, any article or implant provided herein releases (e.g., a free form of) D1 (a steroid) and D2 (a statin, such as a rosuvastatin radical described herein) into the eye of the individual. In some embodiments, the article or implant releases a statin into the eye into the eye of the individual.

In some embodiments, the article or implant releases (into the eye) a compound having the following structure:

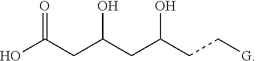

In some embodiments, [symbol] is a single bond or a double bond. In some embodiments, G is substituted heteroaryl or substituted carbocyclyl.

In some embodiments, the article or implant releases (into the eye) a compound having the following structure:

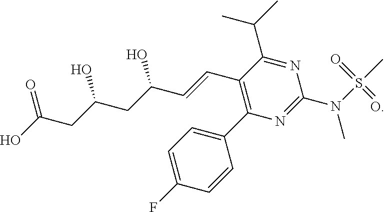 [SMILES: CC(C)c1nc(N(C)S(C)(=O)=O)nc(-c2ccc(F)cc2)c1/C=C/[C@@H](O)C[C@@H](O)CC(=O)O]

In some embodiments, any article or implant provided herein releases the statin or conjugate or compound provided herein for period of one month or more (e.g., two or more months, three or more months, or four or more months).

In some embodiments, any method provided herein comprises intravitreally administering the compound, pharmaceutically acceptable salt, implant, article, or composition into the eye of the individual.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A shows the chemical structure of a steroid-statin compound, rosuvastatin-anecortave (Compound 1), exemplified herein.

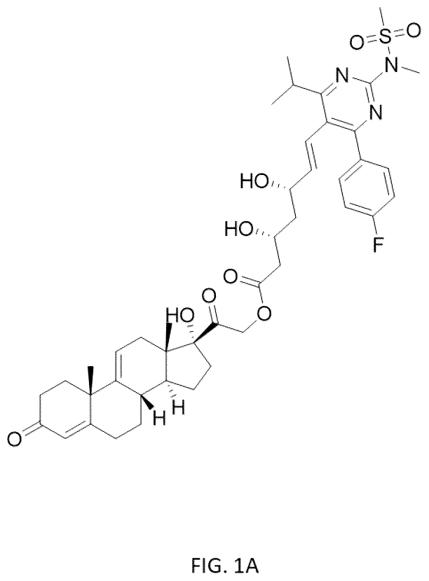

FIG. 1B shows the heat processed pellet of Compound 1.

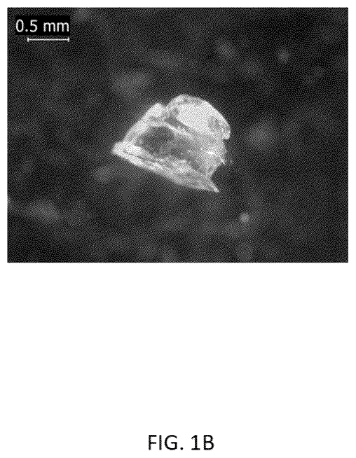

FIG. 1C shows the drug release profile for Compound 1 (pellet) in phosphate-buffered saline (PBS) and in fetal bovine serum (FBS).

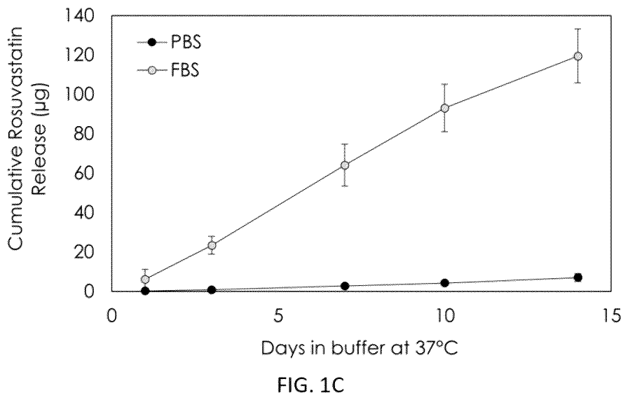

FIG. 1D shows the progression of surface erosion drug release of Compound 1 at day 0 (Panel 1), at day 7 (Panel 2), and at day 14 (Panel 3) in PBS.

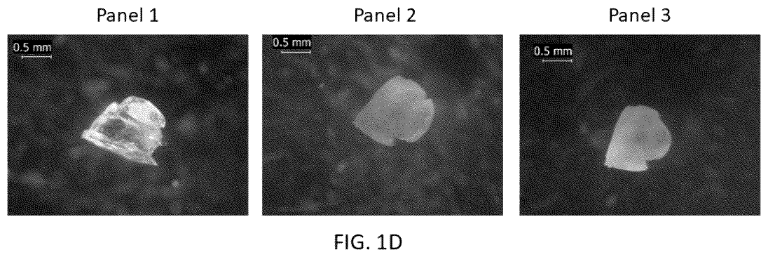

FIG. 1E shows the progression of surface erosion drug release of Compound 1 at day 0 (Panel 1), at day 7 (Panel 2), and at day 14 (Panel 3) in FBS.

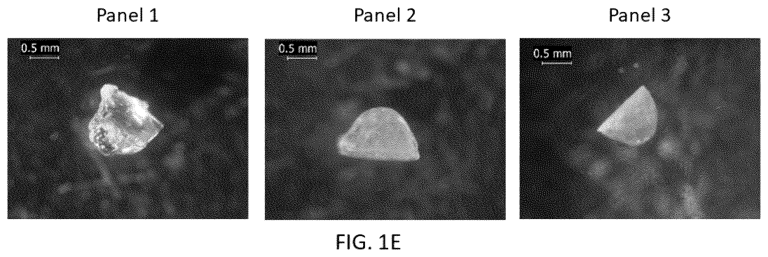

FIG. 2A shows the chemical structure of a steroid-statin compound, rosuvastatin-Gly-anecortave (Compound 2), exemplified herein.

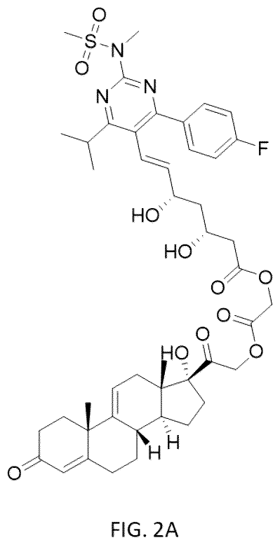

FIG. 2B shows the heat processed pellet of Compound 2.

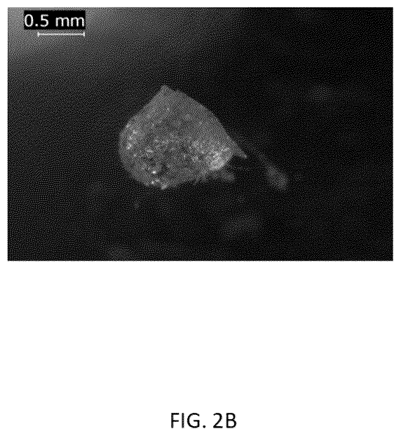

FIG. 2C shows the drug release profile for Compound 2 (pellet) in PBS and in FBS.

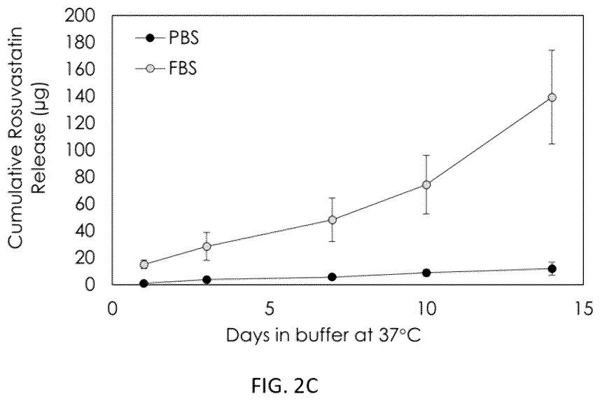

FIG. 2D shows the progression of surface erosion drug release of Compound 2 at day 0 (Panel 1), at day 7 (Panel 2), and at day 14 (Panel 3) in PBS.

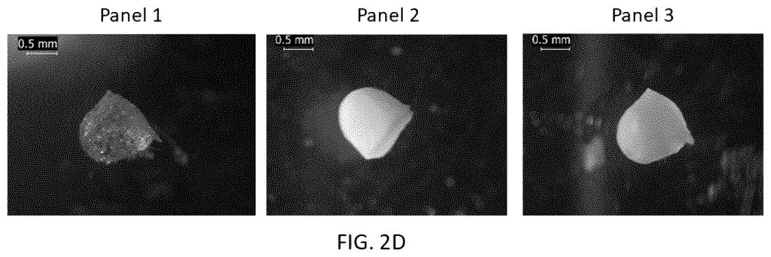

FIG. 2E shows the progression of surface erosion drug release of Compound 2 at day 0 (Panel 1), at day 7 (Panel 2), and at day 14 (Panel 3) in FBS.

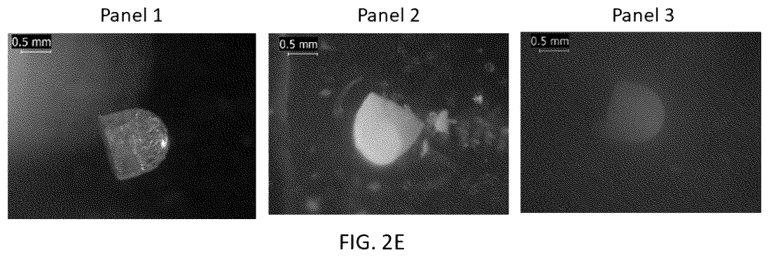

FIG. 3A shows an extruded implant of a steroid-statin compound, rosuvastatin-anecortave (Compound 1), exemplified herein.

FIG. 3B shows the drug release profile for Compound 1 (extruded implant) in PBS and FBS.

FIG. 3C shows the progression of surface erosion drug release of Compound 1 (extruded implant) at day 1 (Panel 1), at day 28 (Panel 2), at day 35 (Panel 3), at day 49 (Panel 4), at day 63 (Panel 5), and at day 112 (Panel 6) in PBS.

FIG. 4A and FIG. 4B show the chemical structure for a prodrug ester and an active pharmaceutical ingredient for a steroid (e.g., anecortave acetate (FIG. 4A) and anecortave desacetate (FIG. 4B), respectively).

FIG. 5 illustrates exemplary insertion locations for various routes of administration.

FIG. 6 illustrates exemplary pathway and targets for treating or modulating ocular disease, such as disease related to retinal pigment epithelium (RPE) cells.

FIG. 7 illustrates exemplary rod implant and near zero order in vitro release profile for the implant over about 150 days.

FIG. 8 illustrates exemplary rod implants that have undergone ocular insertion, specifically a rosuvastatin-anecortave compound having a diameter of 0.2 mm and length of 1 mm.

FIG. 9 illustrates exemplary rod implants having good ocular tolerability after four months.

FIG. 10 shows a relative decrease in levels of (ocular) components of cholesterol biosynthesis pathway after an implant described herein is administered to the eye.

### DETAILED DESCRIPTION OF THE INVENTION

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other some embodiments, for example, an embodiment of any composition of matter, composition, method, or process, or the like, described herein, may “consist of” or “consist essentially of” the described features.

The terms “treat,” “treating,” or “treatment” as used herein, include reducing, alleviating, abating, ameliorating, managing, relieving, or lessening the symptoms associated with a disease, disease state, condition, or indication (e.g., provided herein) in either a chronic or acute therapeutic scenario. Also, treatment of a disease or disease state described herein includes the disclosure of use of such compound or composition for the treatment of such disease, disease state, disorder, or indication.

“Amino” refers to the —NH₂ radical.

“Cyano” refers to the —CN radical.

“Nitro” refers to the —NO₂ radical.

“Oxo” refers to the ═O radical.

“Hydroxyl” refers to the —OH radical.

“Alkyl” generally refers to an acyclic (e.g., straight or branched) or cyclic hydrocarbon (e.g., chain) radical consisting solely of carbon and hydrogen atoms, such as having from one to fifteen carbon atoms (e.g., C₁-C₁₅ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In some embodiments, an alkyl comprises one to thirteen carbon atoms (e.g., C₁-C₁₃ alkyl). In some embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a}(where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In some embodiments, an alkenyl comprises two to eight carbon atoms. In other embodiments, an alkenyl comprises two to four carbon atoms. The alkenyl is optionally substituted as described for “alkyl” groups.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like. Unless stated otherwise specifically in the specification, an alkylene chain is optionally substituted as described for alkyl groups herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. In some instances, an aryl provided herein is a multicyclic ring system, such as a multicyclic ring system of a compound provided in Table 1 or Table 2). The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, ortrifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{C} is a straight or branched alkylene or alkenylene chain, and where each of the above substituents is unsubstituted unless otherwise indicated.

“Aralkyl” or “aryl-alkyl” refers to a radical of the formula —R^{c}-aryl where R^{C} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In some instances, a cycloalkyl provided herein is a fused or bridged ring system, such as a fused or bridged ring system of a compound provided in Table 1 or Table 2). Unless stated otherwise specifically in the specification, “carbocyclyl” and “cycloalkyl” are used interchangeably herein. In some embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a}(where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{C} is a straight or branched alkylene or alkenylene chain, and where each of the above substituents is unsubstituted unless otherwise indicated.

“Carbocyclylalkyl” refers to a radical of the formula —R^{c}-carbocyclyl where R^{C} is an alkylene chain as defined above. The alkylene chain and the carbocyclyl radical is optionally substituted as defined above.

“Carbocyclylalkenyl” refers to a radical of the formula —R^{c}-carbocyclyl where R^{C} is an alkenylene chain as defined above. The alkenylene chain and the carbocyclyl radical is optionally substituted as defined above.

“Carbocyclylalkoxy” refers to a radical bonded through an oxygen atom of the formula —O—R^{c}-carbocyclyl where R^{C} is an alkylene chain as defined above. The alkylene chain and the carbocyclyl radical is optionally substituted as defined above.

“Halo” or “halogen” refers to fluoro, bromo, chloro, or iodo substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a fluoroalkyl, such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. In some embodiments, heteroalkyl includes alkylamino, alkylaminoalkyl, aminoalkyl, heterocycloalkyl, heterocycloalkyl, heterocyclyl, and heterocycloalkylalkyl, as defined herein. Unless stated otherwise specifically in the specification, heteroalkyl does not include alkoxy as defined herein. Unless stated otherwise specifically in the specification, a heteroalkyl group is optionally substituted as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, “heterocyclyl” and “heterocycloalkyl” are used interchangeably herein. Unless stated otherwise specifically in the specification, the heterocyclyl radical is a monocyclic, bicyclic, tricyclic or tetracyclic ring system, which optionally includes fused or bridged ring systems. In some instances, a heterocyclyl provided herein is a fused or bridged ring system (e.g., bicyclic, tricyclic or tetracyclic ring system), such as a fused or bridged ring system of a compound provided in Table 1 or Table 2). The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated. In some instances, the heterocyclyl radical is saturated and substituted. In some instances, the heterocyclyl radical is unsaturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a}(where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. In some instances, an heteroaryl provided herein is a fused or bridged ring system, such as a fused or bridged ring system of a compound provided in Table 1 or Table 2). The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

In general, optionally substituted groups are each independently substituted or unsubstituted. Each recitation of an optionally substituted group provided herein, unless otherwise stated, includes an independent and explicit recitation of both an unsubstituted group and a substituted group (e.g., substituted in some embodiments, and unsubstituted in certain other embodiments). Unless otherwise stated, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl).

The compounds disclosed herein, reference to any atom includes reference to isotopes thereof. For example, reference to H includes reference to any isotope thereof, such as a ¹H, ²H, ³H, or mixtures thereof.

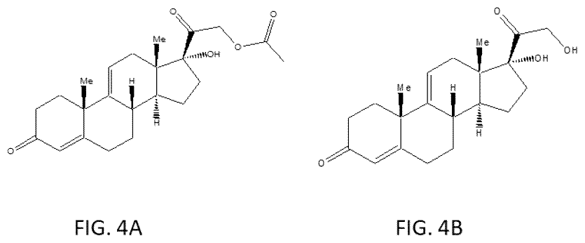

Generally, recitations of “anecortave” herein refer to “anecortave” in the desacetate form of FIG. 4B; however, where applicable, recitations of “anecortave” include disclosure of each of the “desacetate” and “acetate” forms.

The term “pellet,” as used herein, refers to the shape of the pharmaceutical compositions of the disclosure that is rounded, spherical, cylindrical, or a combination thereof. In some embodiments, the pellet has a mean diameter from about 0.2 to 5 mm, e.g., from about 0.2 to 1 mm, from about 0.2 to 2 mm, from about 0.3 to 3 mm, from about 1.5 to 5 mm, from about 2 to 5 mm, from about 2.5 to 5 mm, from about 3 to 5 mm, from about 3.5 to 5 mm, from about 4 to 5 mm, or from about 4.5 to 5 mm.

“Pharmaceutically acceptable salt” includes both acid and base addition salts. A pharmaceutically acceptable salt of any one of the pharmacological agents described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

In some instances, producing a final medicinal product from an active pharmaceutical ingredient (API) that is a solid or a liquid at room temperature is an extensive and costly process. In some instances, pharmaceutical formulation of an API (e.g., that is a liquid at room temperature) require, for example, excipients (e.g., stabilizers, additives, adjuvants, etc.) or conjugation to another molecule (e.g., a polymer) to stabilize and/or produce a processable and/or storable product. In some instances, APIs (e.g., that are solids at room temperature) are used for pharmaceutical formulations, such as avoiding the additional processing and formulation for producing medicinal products from liquid APIs; albeit, still incurring significant cost to formulation to a final medicinal product. In some instances, the cost of formulating solid or liquid APIs as well as the limited processability of liquid or solid APIs limit the efficacy and/or adoption of potentially beneficial therapeutics.

In some instances, patient compliance is an unresolved issue in the clinic. In some instances, modified-release pharmaceuticals improve patient compliance. For example, extended-release (ER) dosage forms, such as sustained-release (SR) or controlled-release (CR) dosage forms, may facilitate compliance with a therapeutic regimen in some instances. In some instances, SR and CR dosage forms are designed to liberate an API at a certain rate, such as to maintain a particular drug concentration over a period of time. For example, SR maintains drug release over a sustained period but not at a constant rate, while CR maintains drug release over a sustained period at a more consistent (e.g., nearly constant) rate (e.g., zero-order). Despite their ability to extend the dosing of an active, such dosage forms can be difficult to develop. In some instances, such dosage forms include controlled release excipients (e.g., polymers) and/or controlled release matrices to facilitate controlled release. In the case of liquid or otherwise low melting point active agents, controlled release formulations, in some instances, are even more difficult to develop. Even in the best circumstances, controlled release forms, in some instances, have limited durations of active release (e.g., 24-hour release windows), so patient compliance remains an issue.

Provided in certain embodiments herein are processable compounds that address the burden of medicinal product formulation as well as patient compliance. In certain embodiments, compounds described herein are solids at body temperature (e.g., about 37° C., or lower). In certain embodiments, compounds provided herein comprise a first group or radical (e.g., a structure provided in any one of Formula (V), Formula (V′), Formula C1, Formula 1C, Formula C2, Formula 2C, or a radical of a compound provided in Table 1) (e.g., covalently) joined (e.g., conjugated) to a second group. In some embodiments, the first group is a radical of Formula (V), Formula (V′), Formula C1, Formula 1C, Formula C2, or Formula 2C, or a radical of a compound provided in Table 1. In some embodiments, the second group is a group that is itself not processable in free form (e.g., has a melting temperature that is higher than its degradation temperature, is generally insoluble or too soluble, such as in aqueous media, or is otherwise not suited for processing). In certain embodiments, the second group is a group that is not processable in dimer form (e.g., when conjugated directly to itself or via a linker, such as described herein). In certain embodiments, the second group is a group that is processable in dimer form (e.g., when conjugated directly to itself or via a linker, such as described herein). In some embodiments, the second group is a group that has a melting point and/or glass transition temperature of less than 50° C., less than 40° C., less than 37° C., or the like. Generally, such compounds, even if solid at room temperature, may not be suitable for use as implants due to the possibility of melting or deformation in a physiological environment. In some embodiments, the compound is formed into an implantable article (e.g., a pellet), such as using methods described herein (e.g., as described in the examples). In some embodiments, the implantable article has a (e.g., zero-order) controlled release rate over an extended period (e.g., 1 week, 2 weeks, 3 weeks, 4 weeks, 6 weeks, 12 weeks, 52 weeks, or more) in an aqueous medium (e.g., a buffer solution, serum, biological environment (e.g., in the eye), in vivo, or the like). In some embodiments, a compound provided herein (or implant comprising such a compound) is administered to an individual suffering an acute or a chronic disease or condition (e.g., as a therapy for the acute or chronic disease or condition) in any suitable manner (e.g., route of administration, such as by implanting or inserting, and/or frequency of dosing), such as a single dose or a series of doses (e.g., once or twice every 1 week, 2 weeks, 3 weeks, 4 weeks, 6 weeks, 12 weeks, 52 weeks, or more).

In certain instances, compounds (e.g., conjugates) provided herein are used to improve treatment options and/or patient compliance for acute diseases and/or disorders. In some instances, processable compounds described herein are used to improve treatment options and/or patient compliance for chronic diseases and/or disorders. In some embodiments, the processable compounds described herein are used to improve treatment options and/or patient compliance for ophthalmological uses.

Provided in some embodiments herein are compounds (e.g., conjugates) that are processable (e.g., into an article or implant). A processable compound is a compound that can be processed with heat or solvent to form a solid, such as with little (e.g., less than 20 wt. %, less than 10 wt. %, or less than 5 wt. %) or no addition of further excipient. In certain instances, the solid prepared following processing is an amorphous solid or a solid having a highly amorphous morphology (e.g., as discussed in more detail herein). In some instances, the solid prepared following processing starts as an amorphous solid, and, upon further processing (e.g., addition to an aqueous environment), the amorphous solid forms a (e.g., partially or substantially) crystalline solid. In some instances, the (e.g., partially or substantially) crystalline solid has suitable mechanical properties, such as to release (e.g., through surface erosion) a compound and/or radical (in its free form) provided herein. In certain instances, a processable compound provided herein is a solid at room (e.g., 20° C.) and/or physiological temperature (e.g., 37° C.). In some instances, the compound is thermally processable, such as having a melt or glass transition temperature of at least 37° C., at least 40° C., at least 50° C., at least 60° C., at least 70° C., at least 100° C., or the like. In some embodiments, it is beneficial for the compound to be solid at room temperature, but processable at a temperature that is not prohibitively high. In some embodiments, a compound provided herein has a melt and/or glass transition temperature of less than 200° C., less than 150° C., less than 140° C., less than 130° C., less than 125° C., less than 120° C., or the like.

In some embodiments, the compounds have any suitable morphology, such as to facilitate processing and/or pharmacodynamic effects (e.g., release profile). In certain embodiments, the compound (or implant or pharmaceutical composition comprising the compound) is amorphous (or comprises a highly amorphous content). In some embodiments, a compound (e.g., morphology) provided herein is a solid, such as at a physiological temperature (e.g., having a melting point (Tₘ) and/or glass transition temperature (T_{g}) of at least 37° C.). In some embodiments, the compound is a crystalline solid, film, glass, or amorphous solid (e.g., at a temperature of at least 37° C.). In some instances, such as when the compound is further processed (e.g., introduced to an aqueous environment), the compound (or composition, article, or implant comprising the compound) provided herein has a crystallinity of 15% or more (e.g., determined by PXRD, DSC, or polarized light microscopy). In some embodiments, the compound (or composition, article, or implant comprising the compound) has a crystallinity of at most 15% (e.g., determined by PXRD, DSC, or polarized light microscopy). In some embodiments, the compound (or composition, article, or implant comprising the compound) has a crystallinity of at most about 15% or less (e.g., determined by PXRD, DSC, or polarized light microscopy) after processing the compound into an article or implant provided herein (e.g., via extrusion processing described herein) but has a crystallinity of 15% or more after further processing (e.g., addition to an aqueous environment). In some embodiments, the compound (or composition, article, or implant comprising the compound) is substantially non-crystalline (e.g., determined by PXRD, DSC, or polarized light microscopy). In some embodiments, the compound (or composition, article, or article comprising the compound) is amorphous (e.g., determined by PXRD, DSC, or polarized light microscopy). In some embodiments, the compound (e.g., morphology) has a thermal melting point (Tₘ) that is greater than or equal to the glass transition temperature (T_{g}). In some embodiments, the compound has a melting point of at least 37° C. In some embodiments, the compound (e.g., morphology) has a melting point of at least 100° C. In some embodiments, either one or both of the first and/or second radicals (or (e.g., active) fragments or metabolites thereof) of the compounds (e.g., drug conjugates) and (e.g., active) agents are released (e.g., in their free form), the release being controlled release and/or extended release. In some embodiments, either one or both of the first and/or second radicals of the compounds and agents are released (e.g., in their free form) for at least 15 days (e.g., in solution, buffer solution, serum, biological environment, in vivo, or the like).

Described in certain embodiments herein are processable agents (e.g., compounds) formed from a processable group (e.g., a radical that makes a non-processable radical processable when linked or joined thereto) and a non-processable moiety (e.g., a radical that, if in its free form, would not be processable, such as by thermal techniques, e.g., because of a melting point that is below a physiological temperature). In some embodiments, the processable agents described herein are processable into a solid (e.g., at a temperature of at least 20° C., 25° C., 30° C., 37° C., or more). In some embodiments, provided herein are compounds useful in therapies for treating acute, chronic, or both disease or condition. In some instances, the conjugates provided herein represent a significant advance in the art, e.g., as processable compounds suitable for being formed into or formulated into controlled and/or extended release articles, or implants, or other pharmaceutical compositions that are beneficial for treating acute and/or chronic diseases or disorders, such as with infrequent (e.g., a single, or weekly, monthly, or less frequent) administration.

Provided in some embodiments herein is a compound having a structure represented by Formula 1: D1-L-D2. In some embodiments, D1 is a radical of a steroid or a radical of a statin. In some embodiments, D1 is a radical of a steroid. In some embodiments, D1 is a radical of a statin. In some embodiments, D2 is a radical of a statin. In some embodiments, L is a linker. In some embodiments, L is a bond. In some embodiments, D1 is described elsewhere herein. In some embodiments, D2 is described elsewhere herein. In some embodiments, L is described elsewhere herein. In some embodiments, the compound is a pharmaceutically acceptable salt.

Provided in some embodiments herein is a compound having a structure represented by Formula I: D1-L-D2. In some embodiments, D1 is a radical of a steroid. In some embodiments, D2 is a radical of a statin. In some embodiments, L is a linker. In some embodiments, L is a bond. In some embodiments, D1 is described elsewhere herein. In some embodiments, D2 is described elsewhere herein. In some embodiments, L is described elsewhere herein. In some embodiments, the compound is a pharmaceutically acceptable salt.

In some embodiments, D1 is a radical of a steroid. In some embodiments, D1 is a radical of an anti-inflammatory steroid (e.g., dexamethasone, hydrocortisone, or triamcinolone). In some embodiments, D1 is a radical of a benign steroid (e.g., anecortave, cholesterol, cholic acid, or deoxycholic acid)). In some instances, a benign steroid is a (steroid) carrier. In some instances, a benign steroid is a steroid that (in its free form) does not provide a significant therapeutic effect and/or benefit (e.g., to an individual (e.g., in need thereof)). In some embodiments, D1 is a radical of an angiostatic steroid (e.g., anecortave). In some embodiments, D1 is a radical of anecortave.

In some embodiments, D1 is a radical of a steroid selected from the group consisting of anecortave (radical), dexamethasone (radical), hydrocortisone (radical), triamcinolone (radical), or prednisolone (radical). In some embodiments, D1 is a radical of dexamethasone. In some embodiments, D1 is a radical of hydrocortisone. In some embodiments, D1 is a radical of anecortave (e.g., anecortave desacetate).

In some embodiments, D1 is a radical of anecortave (e.g., anecortave desacetate).

In some embodiments, D1 is not a radical of hydrocortisone.

In some embodiments, D1 is a radical of a statin (or an active form thereof). In some embodiments, D1 is a radical of a high intensity statin (or an active form thereof). In some embodiments, D1 is a radical of a medium intensity statin (or an active form thereof). In some embodiments, D1 is a radical of a low intensity statin (or an active form thereof). In some instances, the intensity of a statin is determined from the reduction in low-density lipoprotein (LDL) in an individual (e.g., compared to a baseline amount) after administration of the statin. In some embodiments, D1 is a radical of a hydrophilic statin (or an active form thereof). In some instances, a hydrophilic statin enters into a cell via protein transport. In some embodiments, D1 is a radical of a lipophilic statin (or an active form thereof). In some instances, a lipophilic statin (e.g., passively) enters into a cell through the cell membrane. In some embodiments, D1 is a radical of a statin selected from the group consisting of cerivastatin (radical), fluvastatin (radical), pitavastatin (radical), an active (e.g., carboxylate) form of lovastatin (radical), an active (e.g., carboxylate) form of mevastatin (radical), an active (e.g., carboxylate) form simvastatin (radical), and pravastatin (radical). In some embodiments, D1 is a radical of cerivastatin, fluvastatin, or pitavastatin. In some embodiments, D1 is a radical of lovastatin (or an active form thereof), a radical of mevastatin (or an active form thereof), a radical of simvastatin (or an active form thereof), or pravastatin. In some embodiments, D1 is a radical of a compound provided in Table 2.

In some embodiments, D1 is a radical of a carrier. In some embodiments, D1 is a radical of an inactive agent. In some embodiments, D1 is a radical of a non-medicinal agent. In some embodiments, D1 (e.g., the radical of an inactive agent or a non-medicinal agent) is an agent that (in its free form) does not provide a significant, measurable, and/or direct therapeutic effect and/or benefit (e.g., to an individual (e.g., in need thereof)). In some embodiments, D1 (e.g., the radical of an inactive agent or a non-medicinal agent) is an agent that (in its free form) does not provide a therapeutic effect and/or benefit (e.g., to an individual (e.g., in need thereof)). In some embodiments, D1 (the carrier agent) (e.g., in its free form) is not a therapeutically active agent (or drug). In some embodiments, D1 (the carrier agent) (e.g., in its free form) is a therapeutically active agent (or drug). In some embodiments, D1 is a radical of a natural compound. In some embodiments, D1 is naturally occurring in nature. In some embodiments, D1 is naturally occurring in the body.

In some embodiments, D1 comprises a three-ring core structure. In some embodiments, D1 comprises a three-ring core structure that is a processable (carrier) group (e.g., a processable group described herein).

In some embodiments, D1 comprises a core structure with three or more fused rings. In some embodiments, D1 has a 3-ring structure, a 4-ring structure, a 5-ring structure, or a 6-ring structure.

In some embodiments, D1 has a structure represented by Formula (V).

In some embodiments, D1 has a structure represented by Formula (V′).

In some embodiments, D1 has a structure represented Formula (V), Formula (V′), Formula C1, Formula 1C, Formula C2, Formula 2C, or a radical of a compound provided in Table 1

In some embodiments, D1 is any radical (e.g., carrier radical) provided herein (e.g., a radical of any compound provided in Table 1 or Table 2).

In some embodiments, D1 is described elsewhere herein, such as in Table 1 or Table 2.

In some embodiments, D1 is a radical of a compound having a structure represented by a compound provided in Table 1 or Table 2. In some embodiments, D1 is a radical of a compound having a structure represented by a compound provided in Table 1. In some embodiments, D1 is a radical of a compound having a structure represented by a compound provided in Table 2.

In some embodiments, D1 is a radical of an active agent. In some embodiments, D1 is a radical of a therapeutically active agent or a drug. In some embodiments, D1 is a radical of a therapeutically active agent. In some embodiments, D1 is a radical of a drug.

In some embodiments, D1 has a structure represented by Formula B1, Formula 1B, Formula B1′, Formula 1B′, Formula B1″, Formula 1B′″, Formula B2, Formula 2B, Formula B2′, Formula 2B′, Formula B2″, Formula 2B″, or a radical of a compound provided in Table 2.

In some embodiments, D2 is a radical of an active agent. In some embodiments, D2 is a radical of a therapeutically active agent or a drug. In some embodiments, D2 is a radical of a therapeutically active agent. In some embodiments, D2 is a radical of a drug.

In some embodiments, D2 is a radical of a statin described herein. In some embodiments, D2 is a radical of a statin selected from the group consisting of atorvastatin (radical), rosuvastatin (radical), cerivastatin (radical), fluvastatin (radical), pitavastatin (radical), an active (e.g., carboxylate) form of lovastatin (radical), an active (e.g., carboxylate) form of mevastatin (radical), an active (e.g., carboxylate) form simvastatin (radical), and pravastatin (radical). In some embodiments, D2 is a radical of a statin selected from the group consisting of cerivastatin (radical), fluvastatin (radical), pitavastatin (radical), lovastatin (radical), mevastatin (radical), simvastatin (radical), and pravastatin (radical). In some embodiments, D2 is a radical of atorvastatin, rosuvastatin, cerivastatin, fluvastatin, or pitavastatin. In some embodiments, D2 is a radical of cerivastatin, fluvastatin, or pitavastatin. In some embodiments, D2 is a radical of lovastatin, mevastatin, simvastatin, or pravastatin.

In some embodiments, D2 is a radical of rosuvastatin. In some embodiments, D2 is a carboxylate radical of rosuvastatin. In some embodiments, D2 is a carbonyl radical of rosuvastatin.

In some embodiments, D2 has a structure represented by Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2.

In some embodiments, D1 and D2 have a structure represented by Formula (V), Formula (V′), Formula (V), Formula (V′), Formula C1, Formula 1C, Formula C2, Formula 2C, Formula A, Formula A′, Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 1 or Table 2.

In some embodiments, D2 has a structure represented by Formula A or Formula A′. In some embodiments, D2 is a radical of any therapeutic agent (or drug) provided herein.

In some embodiments, D2 is any therapeutic agent (or drug or form) provided in Table 2.

In some embodiments, D2 is described elsewhere herein, such as in Table 2.

In some embodiments, D2 is a radical of a compound having a structure represented by a compound provided in Table 2.

Provided in some embodiments herein is a compound having a structure represented by Formula II: D1-L-D2. In some embodiments, D1 is a radical of a steroid. In some embodiments, D1 is a radical of anecortave, such as a (e.g., hydroxyl) radical of anecortave described herein. In some embodiments, D2 is a radical of rosuvastatin, such as a (e.g., carboxylate) radical of rosuvastain described herein. In some embodiments, L is a linker. In some embodiments, L is a bond. In some embodiments, D1 is described elsewhere herein. In some embodiments, D2 is described elsewhere herein. In some embodiments, L is described elsewhere herein. In some embodiments, the compound is a pharmaceutically acceptable salt.

In some instances, the linker (e.g., L) is a diradical. In some instances, the diradical is a molecular species (e.g., an organic compound) with two electrons occupying degenerate molecular orbitals.

In some embodiments, the linker (e.g., L) comprises one or more linker groups, each linker group being independently selected from any linker or linker group provided herein (e.g., any linker or linker group provided in Table 3).

In some embodiments, the linker (e.g., L) is any linker or linker group provided herein (e.g., any linker or linker group provided in Table 3).

In some embodiments, L (the linker) is described elsewhere herein, such as in Table 3.

In some embodiments, L (the linker) is a bond, substituted or unsubstituted alkyl, substituted or unsubstituted heteroalkyl, substituted or unsubstituted alkoxy, substituted or unsubstituted carbocyclyl, or substituted or unsubstituted heterocyclyl.

In some embodiments, L is a bond.

In some embodiments, L (the linker) is substituted or unsubstituted alkyl, substituted or unsubstituted heteroalkyl, substituted or unsubstituted alkoxy, substituted or unsubstituted carbocyclyl, or substituted or unsubstituted heterocyclyl.

In some embodiments, the linker (e.g., L) comprises substituted or unsubstituted alkyl.

In some embodiments, the linker (e.g., L) is substituted or unsubstituted alkyl.

In some embodiments, the linker (e.g., L) is substituted alkyl.

In some embodiments, the linker (e.g., L) comprises alkyl substituted with oxo. In some embodiments, the linker (e.g., L) is alkyl substituted with oxo.

In some embodiments, the linker (e.g., L) comprises —(O═C)CH₂—. In some embodiments, the linker (e.g., L) is —(O═C)CH₂—.

In some embodiments, the linker (e.g., L) is or comprises -(Q¹-M-Q²)-. In some embodiments, Q¹ and Q² are each independently absent or (C═X¹)X². In some embodiments, X¹ is O or S. In some embodiments, X² is O, S, or NR¹. In some embodiments, R¹ is hydrogen or C₁-C₆ alkyl. In some embodiments, M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl).

In some embodiments, Q¹ and Q² are each independently absent, C═O, C═S, (C═O)O, (C═O)S, (C═O)N, or (C═S)S.

In some embodiments, Q¹ and Q² are described elsewhere herein.

In some embodiments, X¹ and X² are described elsewhere herein.

In some embodiments, R¹ is described elsewhere herein.

In some embodiments, M is substituted or unsubstituted alkyl (e.g., C₁-C₆ alkyl), substituted or unsubstituted heteroalkyl (e.g., C₁-C₆ heteroalkyl), or substituted or unsubstituted aryl. In some embodiments, M is substituted or unsubstituted alkyl (e.g., C₁-C₆ alkyl (e.g., alkyl-carbocyclyl-alkyl)) or substituted or unsubstituted heteroalkyl (e.g., C₁-C₆ heteroalkyl).

In some embodiments, M is described elsewhere herein.

In some embodiments, D1 is a (e.g., hydroxyl) radical of a compound provided in Table 1.

TABLE 1

| Name | Structure |
|---|---|
| Hydrocortisone | 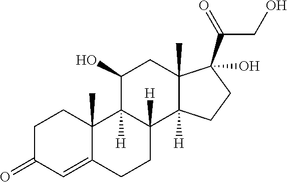 |
| Dexamethasone | 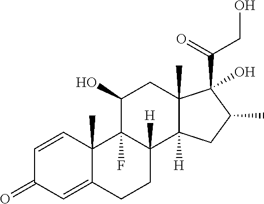 |
| Anecortave desacetate | 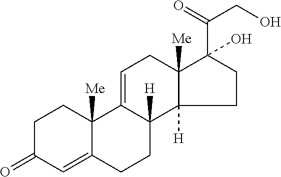 |
| Triamcinolone | 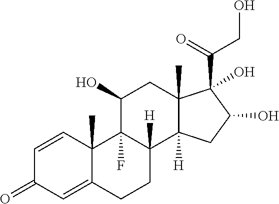 |
| Prednisolone | 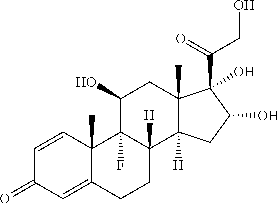 |

In some embodiments, D1 is a (e.g., carboxyl) radical of a compound provided in Table 2.

In some embodiments, D2 is a (e.g., carboxyl) radical of a compound provided in Table 2.

In some embodiments, D1 and D2 are each independently a (e.g., hydroxyl or carboxyl) radical of a compound provided in Table 2. In some embodiments, D1 is a steroid radical (e.g., a hydroxyl radical, a carboxylate radical, or a phosphate radical of a steroid) and D2 is a (e.g., a carboxyl) radical of a compound provided in Table 2.

TABLE 2

| Name | Structure |
|---|---|
| Cerivastatin | 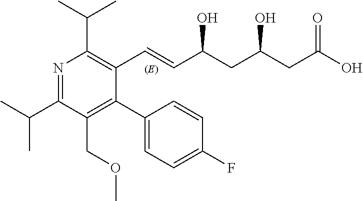 [SMILES: COCc1c(C(C)C)nc(C(C)C)c(/C=C/[C@@H](O)C[C@@H](O)CC(=O)O)c1-c1ccc(F)cc1] |
| Fluvastatin | 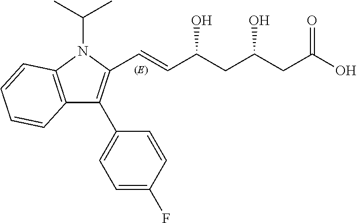 |
| Pitavastatin | 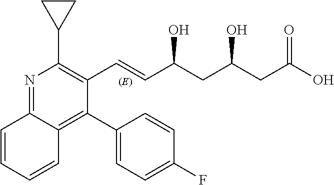 [SMILES: O=C(O)C[C@H](O)C[C@H](O)/C=C/c1c(C2CC2)nc2ccccc2c1-c1ccc(F)cc1] |
| Lovastatin (active form) | 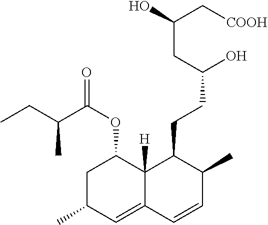 [SMILES: CC[C@H](C)C(=O)O[C@H]1C[C@@H](C)C=C2C=C[C@H](C)[C@H](CC[C@@H](O)C[C@@H](O)CC(=O)O)[C@H]21] |
| Mevastatin (active form) | 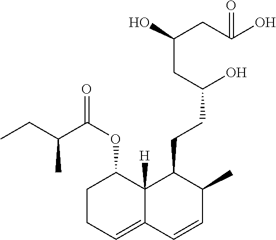 [SMILES: CC[C@H](C)C(=O)O[C@H]1CCC=C2C=C[C@H](C)[C@H](CC[C@@H](O)C[C@@H](O)CC(=O)O)[C@H]21] |
| Simvastatin (active form) | 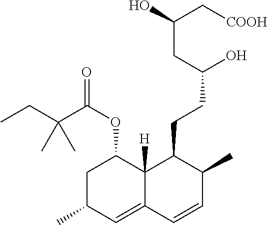 [SMILES: CCC(C)(C)C(=O)O[C@H]1C[C@@H](C)C=C2C=C[C@H](C)[C@H](CC[C@@H](O)C[C@@H](O)CC(=O)O)[C@H]21] |
| Pravastatin | 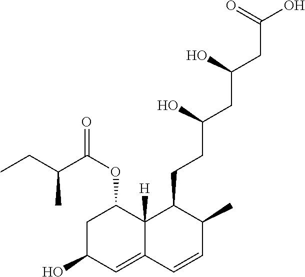 [SMILES: CC[C@H](C)C(=O)O[C@H]1C[C@H](O)C=C2C=C[C@H](C)[C@H](CC[C@@H](O)C[C@@H](O)CC(=O)O)[C@H]21] |
| Atorvastatin | 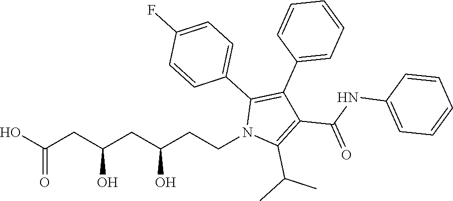 [SMILES: CC(C)c1c(C(=O)Nc2ccccc2)c(-c2ccccc2)c(-c2ccc(F)cc2)n1CC[C@@H](O)C[C@@H](O)CC(=O)O] |
| Rosuvastatin | 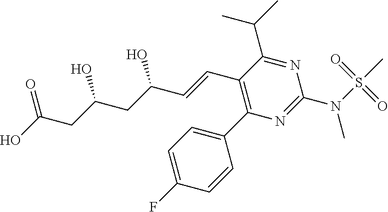 [SMILES: CC(C)c1nc(N(C)S(C)(=O)=O)nc(-c2ccc(F)cc2)c1/C=C/[C@@H](O)C[C@@H](O)CC(=O)O] |

In some embodiments, D1 is a steroid radical (e.g., a hydroxyl radical, a carboxylate radical, or a phosphate radical of a steroid) and D2 is a (e.g., a carboxyl) radical of rosuvastatin.

In some embodiments, D1 is a radical of a compound represented by a structure:

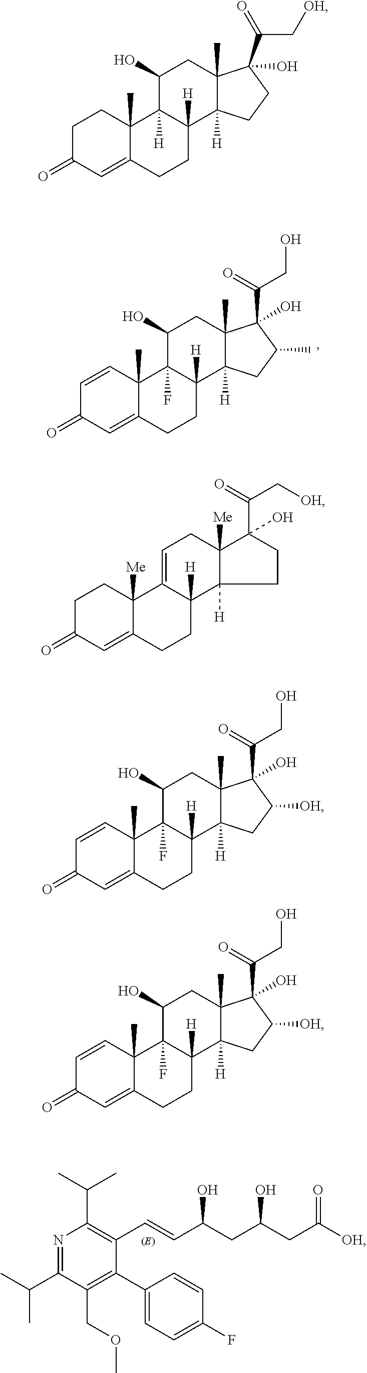

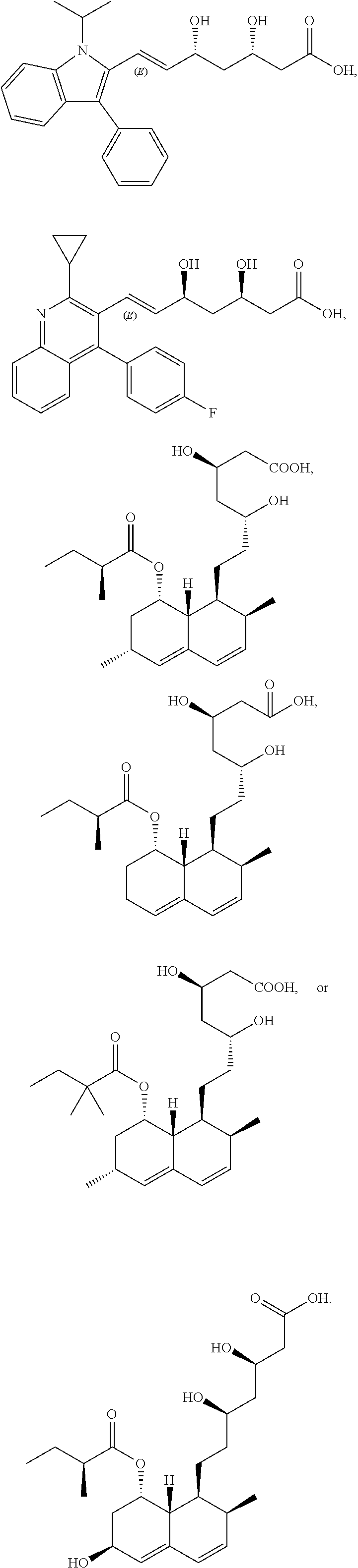

In some embodiments, D1 is a (e.g., hydroxyl) radical of a compound represented by a structure:

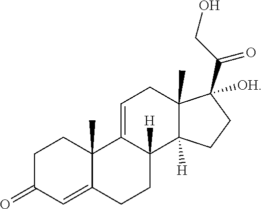

In some embodiments, D1 is a (e.g., carbon) radical of a compound represented by a structure:

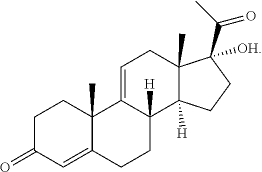

In some embodiments, D1 has the following structure:

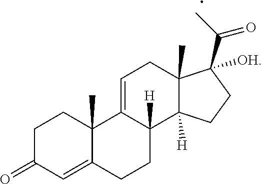

In some embodiments, D1 and D2 are each independently a radical of a compound represented by a structure:

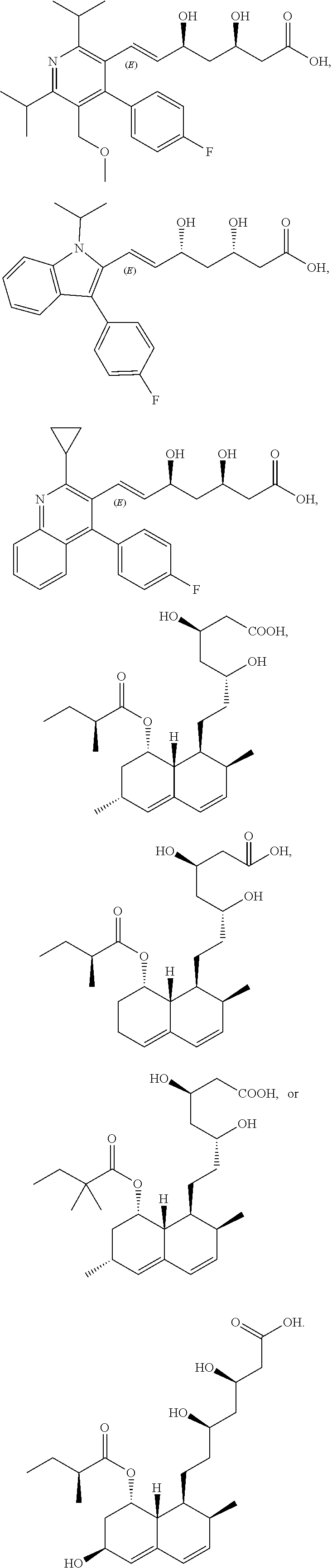

In some embodiments, D1 is a radical of a compound represented by a structure:

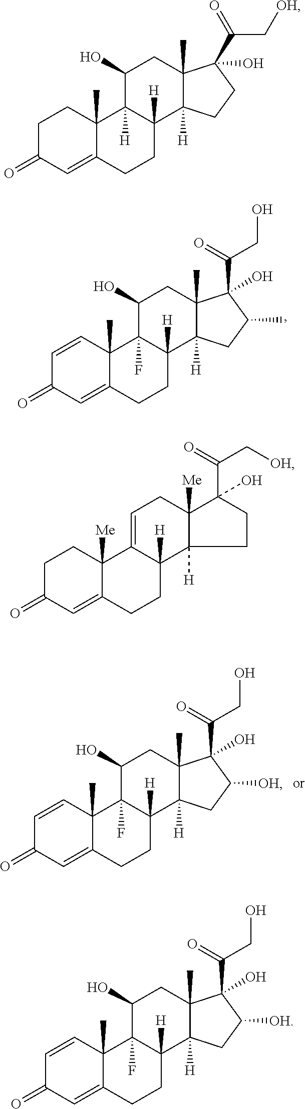

In some embodiments, D2 is a radical of a compound represented by a structure:

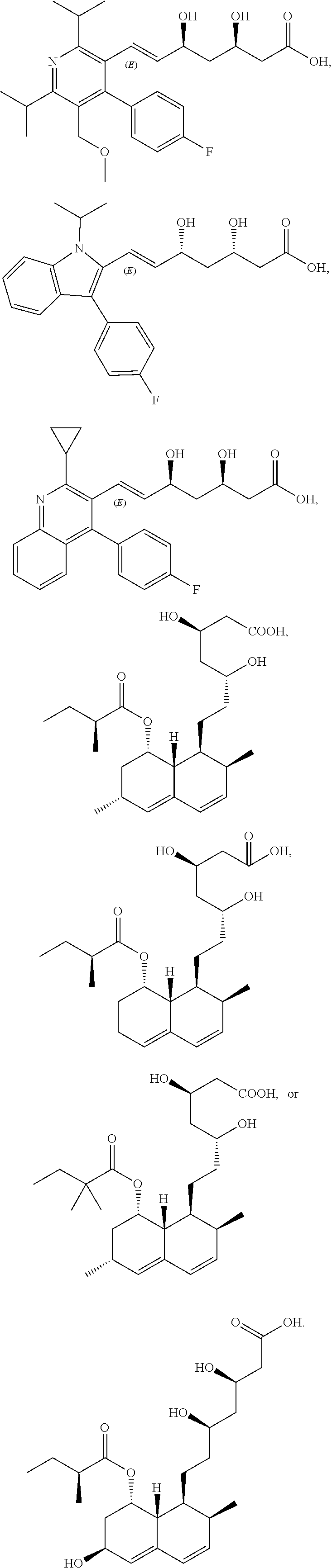

In some embodiments, D2 is a (e.g., hydroxyl) radical of a compound represented by a structure:

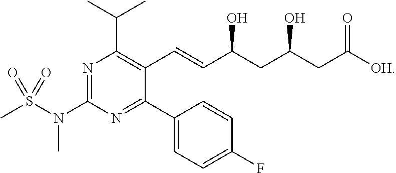

In some embodiments, D2 has the following structure:

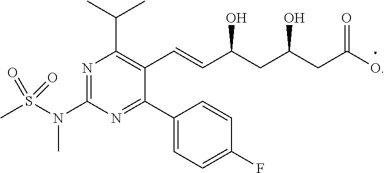

In some embodiments, D2 is a (e.g., carbonyl) radical of a compound represented by a structure:

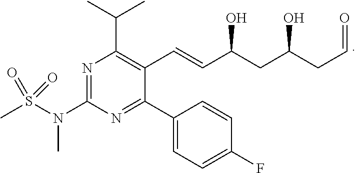

In some embodiments, D2 has the following structure:

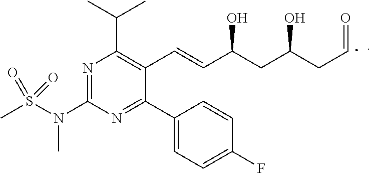

In some embodiments, the linker (e.g., L) comprises or is represented by a structure provided in Table 3. In some embodiments, the linker (e.g., L) comprises or is a diradical derived from a compound (e.g., compound name) provided in Table 3. The linkers in Table 3 are intended to provide examples of linkers described in the disclosure. Other linkers, such as described elsewhere here (a bond, —(O═C)CH₂—, etc.), are also contemplated.

TABLE 3

| Structure | Derived from [compound name] |
|---|---|
| 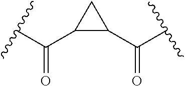 [SMILES: *C(=O)C1CC1C(*)=O] | cyclopropane-1,2-dicarboxylic acid |
| 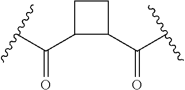 [SMILES: *C(=O)C1CCC1C(*)=O] | cyclobutane-1,2-dicarboxylic acid |
| 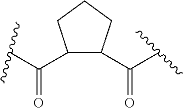 [SMILES: *C(=O)C1CCCC1C(*)=O] | cyclopentane-1,2-dicarboxylic acid |
| 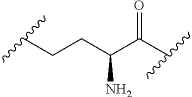 [SMILES: *CC[C@H](N)C(*)=O] | homoserine |
| 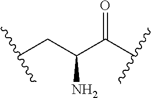 [SMILES: *C[C@H](N)C(*)=O] | serine |
| 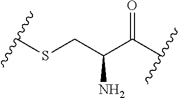 [SMILES: *SC[C@H](N)C(*)=O] | cysteine |
| 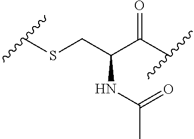 [SMILES: *SC[C@H](NC(C)=O)C(*)=O] | N-acetyl cysteine |
| 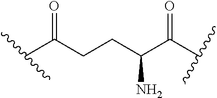 [SMILES: *C(=O)CC[C@H](N)C(*)=O] | glutamic acid |
| 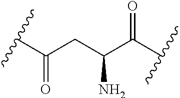 [SMILES: *C(=O)C[C@H](N)C(*)=O] | aspartic acid |
| 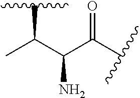 [SMILES: *C(=O)[C@@H](N)[C@H](*)C] | threonine |
| 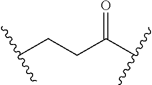 [SMILES: *CCC(*)=O] | 3-hydroxypropanoic acid |
| 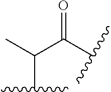 [SMILES: *C(=O)C(*)C] | lactic acid |
| 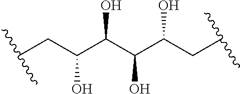 [SMILES: *C[C@@H](O)[C@@H](O)[C@H](O)[C@H](O)C*] | mannitol |
| 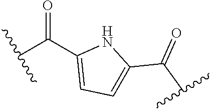 [SMILES: *C(=O)c1ccc(C(*)=O)[nH]1] | 2,5-pyrroledicarboxylic acid |
| 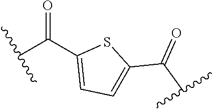 [SMILES: *C(=O)c1ccc(C(*)=O)s1] | 2,5-Thiophenedicarboxylic acid |
| 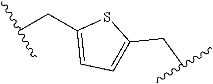 [SMILES: *Cc1ccc(C*)s1] | thiophene-2,5-diyldimethanol |
| 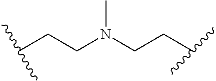 [SMILES: *CCN(C)CC*] | N-Methyldiethanolamine |
| 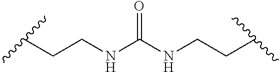 [SMILES: *CCNC(=O)NCC*] | 1,3-Bis(2-hydroxyethyl)urea |
| 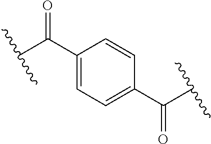 [SMILES: *C(=O)c1ccc(C(*)=O)cc1] | terephthalic acid |
| 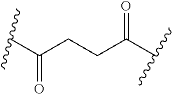 [SMILES: *C(=O)CCC(*)=O] | succinic acid |
| 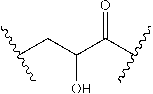 [SMILES: *CC(O)C(*)=O] | Glyceric acid |
| 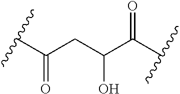 [SMILES: *C(=O)CC(O)C(*)=O] | Malic acid |
| 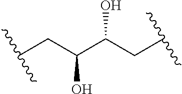 [SMILES: *C[C@@H](O)[C@@H](O)C*] | Erythritol |
| 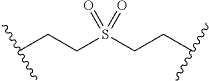 [SMILES: *CCS(=O)(=O)CC*] | BIS(2-HYDROXYETHYL)SULFONE |
| 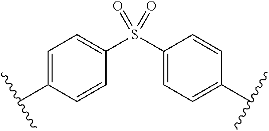 [SMILES: *c1ccc(S(=O)(=O)c2ccc(*)cc2)cc1] | 4,4′-Sulfonyldiphenol |
| 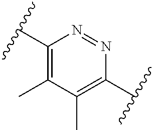 [SMILES: *c1nnc(*)c(C)c1C] | 4,5-Dimethylpyridazine-3,6-diol |
| 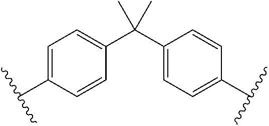 [SMILES: *c1ccc(C(C)(C)c2ccc(*)cc2)cc1] | Bisphenol A |
| 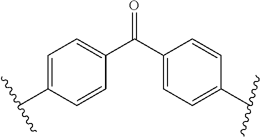 [SMILES: *c1ccc(C(=O)c2ccc(*)cc2)cc1] | 4,4′-Dihydroxybenzophenone |
| 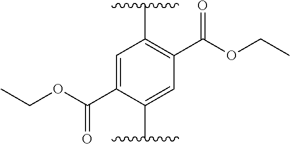 [SMILES: *c1cc(C(=O)OCC)c(*)cc1C(=O)OCC] | Diethyl 2,5-dihydroxyterephthalate |
| 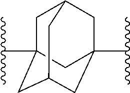 [SMILES: *C12CC3CC(C1)CC(*)(C3)C2] | 1,3-Adamantanediol |
| 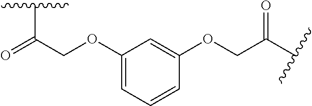 [SMILES: *C(=O)COc1cccc(OCC(*)=O)c1] | Resorcinol-o,o′-diacetic acid |
| 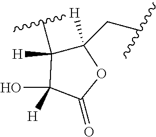 [SMILES: *C[C@H]1OC(=O)[C@H](O)[C@@H]1*] | D-(+)-Ribonic gamma-lactone |
| 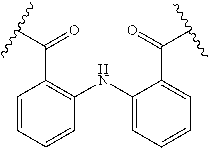 [SMILES: *C(=O)c1ccccc1Nc1ccccc1C(*)=O] | 2,2′-Iminodibenzoic acid |
| 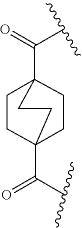 [SMILES: *C(=O)C12CCC(C(*)=O)(CC1)CC2] | Bicyclo[2.2.2]octane-1,4-dicarboxylic acid |
| 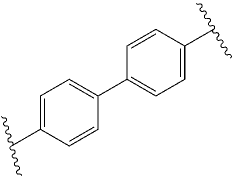 [SMILES: *c1ccc(-c2ccc(*)cc2)cc1] | 4,4′-Dihydroxybiphenyl |
| 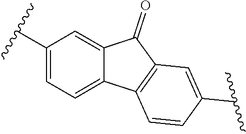 [SMILES: *c1ccc2c(c1)C(=O)c1cc(*)ccc1-2] | 2,7-Dihydroxy-9-fluorenone |
| 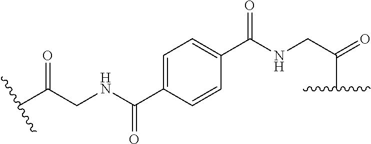 [SMILES: *C(=O)CNC(=O)c1ccc(C(=O)NCC(*)=O)cc1] | 2-[[4- (Carboxymethylcarbamoyl)benzoyl]amino] acetic acid |
| 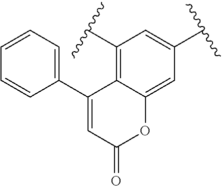 [SMILES: *c1cc(*)c2c(-c3ccccc3)cc(=O)oc2c1] | 5,7-Dihydroxy-4-phenylcoumarin |
| 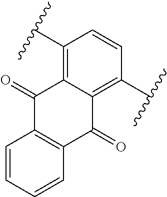 [SMILES: *c1ccc(*)c2c1C(=O)c1ccccc1C2=O] | Quinizarin |
| 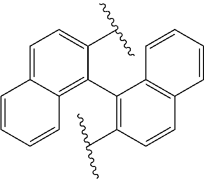 [SMILES: *c1ccc2ccccc2c1-c1c(*)ccc2ccccc12] | 1,1′-Bi-2-naphthol |
| 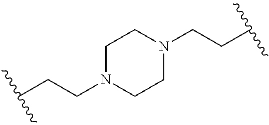 [SMILES: *CCN1CCN(CC*)CC1] | 1,4-Bis(2-hydroxyethyl)piperazine |
| 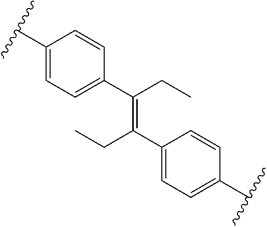 [SMILES: *c1ccc(/C(CC)=C(\CC)c2ccc(*)cc2)cc1] | Diethylstilbestrol, mixture of cis and trans |
| 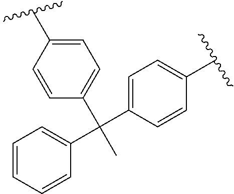 [SMILES: *c1ccc(C(C)(c2ccccc2)c2ccc(*)cc2)cc1] | 4,4′-(1-Phenylethylidene)bisphenol |
| 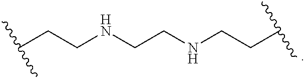 [SMILES: *CCNCCNCC* ; "."] | N,N′-Bis(2-hydroxyethyl)ethylenediamine |
| 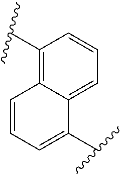 [SMILES: *c1cccc2c(*)cccc12] | 1,5-Dihydroxynaphthalene |
| 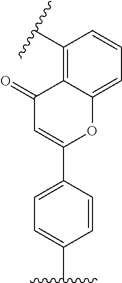 [SMILES: *c1ccc(-c2cc(=O)c3c(*)cccc3o2)cc1] | 41,5-Dihydroxyflavone |
| 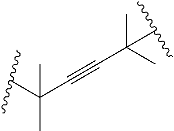 [SMILES: *C(C)(C)C#CC(*)(C)C] | 2,5-Dimethyl-3-hexyne-2,5-diol |
| 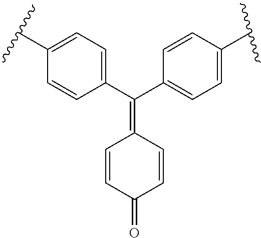 [SMILES: *c1ccc(C(=C2C=CC(=O)C=C2)c2ccc(*)cc2)cc1] | p-Rosolic acid |
| 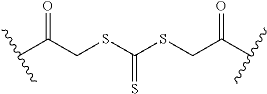 [SMILES: *C(=O)CSC(=S)SCC(*)=O] | Bis(carboxymethyl)trithiocarbonate |
| 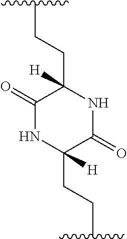 [SMILES: *CC[C@@H]1NC(=O)[C@H](CC*)NC1=O] | cis-3,6-Bis(2-hydroxyethyl)piperazine-2,5- dione |
| 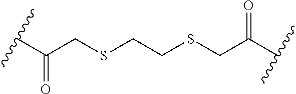 [SMILES: *C(=O)CSCCSCC(*)=O] | 2,2′-(Ethylenedithio)diacetic acid |
| 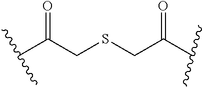 [SMILES: *C(=O)CSCC(*)=O] | 2,2′-Thiodiacetic acid |
| 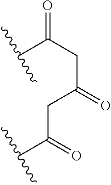 [SMILES: *C(=O)CC(=O)CC(*)=O] | 1,3-Acetonedicarboxylic acid |
| 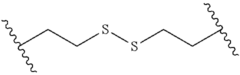 [SMILES: *CCSSCC*] | 2-Hydroxyethyl disulfide |

In some embodiments, D1 is a radical of any compound provided in Table 1 or Table 2, D2 is a radical of any compound provided in Table 2, and the linker (e.g., L) is or comprises one or more linker groups, each linker group being independently selected from any linker or linker group provided in Table 3.

In some embodiments, D1 is a radical of an angiostatic steroid (e.g., anecortave).

In some embodiments, D1 is a radical of a benign steroid (e.g., anecortave or cholesterol).

In some embodiments, D1 is a radical of a corticosteroid (e.g., glucocorticoid or mineral corticoid), a sex steroid, a neurosteroid, an amino steroid, or a secosteroid. In some embodiments, D1 is a radical of a corticosteroid.

In some embodiments, D1 is a radical of dexamethasone.

In some embodiments, D1 is a radical of hydrocortisone.

In some embodiments, D1 is not a radical of hydrocortisone.

In some embodiments, D1 is a radical of anecortave (e.g., anecortave desacetate).

In some embodiments, D2 is a radical of rosuvastatin, wherein the radical is on a carboxylate of rosuvastatin. In some embodiments, D2 is a radical of atorvastatin, wherein the radical is on a carbonyl carbon of rosuvastatin.

In some embodiments, D2 is a radical of a statin or an active form of a statin. In some embodiments, D2 is a radical of a statin. In some embodiments, D2 is a radical of an active form of a statin. In some embodiments, D2 is a radical of cerivastatin, fluvastatin, pitavastatin, lovastatin, mevastatin, simvastatin, pravastatin, or an active form thereof.

In some embodiments, D2 is not a radical of simvastatin, atorvastatin, or rosuvastatin (or an active form thereof).

In some embodiments, D2 is not a radical of simvastatin (or an active form thereof).

In some embodiments, D2 is not a radical of atorvastatin (or an active form thereof).

In some embodiments, D2 is not a radical of rosuvastatin (or an active form thereof).

In some embodiments, D2 is a radical of a statin or an active form thereof, wherein the radical is on a carboxylate of the statin or the active form thereof. In some embodiments, D2 is a radical of an active form of a statin, wherein the radical is on a carboxylate of the active form of the statin.

In some embodiments, L is a bond or a linker.

In some embodiments, L is a bond.

In some embodiments, L is a linker. In some embodiments, L is a hydrolyzable linker. In some embodiments, L is a linker described herein

In some embodiments, L comprises one or more linker group, each linker group being independently selected from the group consisting of a bond, substituted or unsubstituted alkyl, substituted or unsubstituted heteroalkyl, substituted or unsubstituted alkoxy, substituted or unsubstituted aryl, or L is substituted or unsubstituted heteroaryl. In some embodiments, L comprises one or more linker group, each linker group being independently selected from the group consisting of substituted or unsubstituted alkyl, substituted or unsubstituted heteroalkyl, substituted or unsubstituted alkoxy, substituted or unsubstituted aryl, or L is substituted or unsubstituted heteroaryl. In some embodiments, L is substituted or unsubstituted alkyl, substituted or unsubstituted heteroalkyl, substituted or unsubstituted alkoxy, or substituted or unsubstituted aryl. In some embodiments, L is a bond. In some embodiments, L is substituted or unsubstituted alkyl. In some embodiments, L is substituted or unsubstituted heteroalkyl. In some embodiments, L is substituted or unsubstituted alkoxy. In some embodiments, L is substituted or unsubstituted aryl. In some embodiments, L is substituted or unsubstituted heteroaryl.

In some embodiments, the linker (e.g., L) is a bond.

In some embodiments, the linker (e.g., L) is not a bond.

In some embodiments, the linker (e.g., L) is attached to any hydroxyl group of any agent (e.g., D1 or D2) provided herein, such as a hydroxyl, a carboxylate, a phosphate, or an enolizable ketone of any agent (e.g., D1 or D2) provided herein.

In some embodiments, the linker (e.g., L) is attached to any thiol or hydroxyl group of any agent (e.g., D1 or D2) provided herein, such as a thiol, a hydroxyl, or a carboxylate of any agent (e.g., D1 or D2) provided herein.

In some embodiments, the linker (e.g., L) is attached to hydroxyl group of any agent (e.g., D1 or D2) provided herein, such as a hydroxyl of any agent (e.g., D1 or D2) provided herein.

In some instances, the carboxylate (radical) of an agent (e.g., a radical of an agent) provided herein is a carboxylate (radical) of D1 or D2, such that the point of attachment of D1 or D2 to the linker (e.g., L) is through a carboxylate (radical).

In some instances, the hydroxyl (radical) of an agent (e.g., a radical of an agent) provided herein is a hydroxyl (radical) of D1 or D2, such that the point of attachment of D1 or D2 to the linker (e.g., L) is through a hydroxyl (radical).

In some embodiments, the linker (e.g., L) is attached to any thiol group of any agent (e.g., D1 or D2) provided herein.

In some instances, the thiol (radical) of an agent (e.g., a radical of an agent) provided herein is a thiol (radical) of D1 or D2, such that the point of attachment of D1 or D2 to the linker (e.g., L) is through a thiol (radical).

In some embodiments, either or both of D1 or D2 are attached to L through a hydroxyl radical of the D1 or D2.

In some embodiments, D1 and D2 are attached to L through a hydroxyl radical of the D1 or D2.

In some embodiments, D1 and D2 are attached to L through a hydroxyl radical of the D1 or D2.

Unless stated specifically otherwise herein, each instance of radical indicates that a hydrogen (i.e., a hydrogen radical (H•)) is removed from a free form of a compound provided herein, such as any agent described herein (steroid, statin, etc.). In some instances, the radical is a hydroxyl radical. In some instances, the removal of the hydrogen radical from the compound provided herein, such as any agent (steroid, statin, etc.) described herein, provides a radical of an agent (steroid, statin, etc.) that is taken together with any point of a linker provided herein (e.g., L) to form a bond (e.g., between the linker and the radical of the agent).

In some instances, the removal of the hydrogen radical from the compound provided herein, such as any steroid described herein, provides a radical of a steroid that is taken together with any point of a linker provided herein (e.g., L) to form a bond (e.g., between the linker and the steroid radical).

In some instances, the removal of the hydrogen radical from the compound provided herein, such as any statin (e.g., rosuvastatin) described herein, provides a radical of a statin (e.g., rosuvastatin) that is taken together with any point of a linker provided herein (e.g., L) to form a bond (e.g., between the linker and the statin (e.g., rosuvastatin) radical).

Provided in some embodiments herein is a compound having a structure represented by Formula (ID): D1-L-D2. In some embodiments, D1 is a steroid radical. In some embodiments, D2 is a statin radical. In some embodiments, L is a bond. In some embodiments, L is -(Q¹-M-Q²)-. In some embodiments, Q¹ and Q² are each independently absent or (C═X¹)X². In some embodiments, X¹ is O or S. In some embodiments, X² is O, S, or NR¹. In some embodiments, M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl). In some embodiments, the compound is a pharmaceutically-acceptable salt or solvate.

In some embodiments, D1 is described elsewhere herein.

In some embodiments, D2 is described elsewhere herein.

In some embodiments, L is described elsewhere herein.

In some embodiments, the linker (e.g., L) is attached to any hydroxyl group of any steroid provided herein, such as a hydroxyl (e.g., at the C11-, C17-, C21-position), a carboxylate, a phosphate, or an enolizable ketone (e.g., at the C1-position) of any steroid provided herein.

In some embodiments, the linker (e.g., L) is attached to the carboxylate group of any statin (or active form thereof) provided herein, such as a rosuvastatin radical provided herein.

In some embodiments, either or both of D1 or D2 are attached to L through a hydroxyl or carboxyl radical of the D1 or D2.

In some embodiments, D1 and D2 are attached to L through a hydroxyl radical of the D1 or D2.

In some embodiments, D1 and D2 are attached to L through a carboxyl radical of the D1 or D2.

In some embodiments, D1 and D2 are attached to L through a carbonyl radical of the D1 or D2.

In some embodiments, D1 and D2 are attached to L through a hydroxyl radical of the D1 or D2.

In some embodiments, D1 and D2 are attached to L through a carboxylate radical of the D1 or D2.

In some embodiments, provided herein is a compound comprising a steroid radical and a statin radical, the steroid radical comprising a structure of Formula (V):

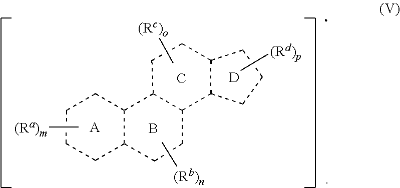

In some embodiments, [symbol] is a single bond or a double bond). In some embodiments, each R^{a}, R^{b}, R^{c}, and R^{d} are independently selected from the group consisting of oxo, halogen, —CN, —NO₂, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, or heterocycloalkyl is optionally substituted. In some embodiments, each of m, n, o, and p are independently 0-6. In some embodiments, any one of R^{a}, R^{b}, R^{c}, and R^{d} are taken together with another of R^{a}, R^{b}, R^{c}, and R^{d} to form a substituted or an unsubstituted cycloalkyl or heterocycloalkyl. In some embodiments, the statin radical is a radical of an active form of a statin described herein, such as a form of rosuvastatin described herein. Also provided in certain embodiments herein are pharmaceutical salts or solvates of a compound of Formula (V).

In some embodiments, Ring B of Formula (V) is an optionally substituted cycloalkyl. In some embodiments, Ring B of Formula (V) does not comprise a heteroatom within the ring (e.g., Ring B is optionally substituted cycloalkyl). In some embodiments, Ring B of Formula (V) comprises only single bonds. In some embodiments, Ring B of Formula (V) comprises at least one double bond. In some embodiments, Ring B of Formula (V) is attached to at least one ring (e.g., Ring A and/or Ring C) that comprises at least one double bond. In some embodiments, Ring A comprises at least one double bond. In some embodiments, Ring C comprises at least one double bond. In some embodiments, Ring A and Ring C each independently comprise at least one double bond. In some embodiments, Ring B of Formula (V) is attached to at least one ring (e.g., Ring A and or Ring C) that is aromatic.

In some embodiments, m is 4. In some embodiments, m is 3. In some embodiments, m is 2. In some embodiments, m is 1. In some embodiments, n is 3. In some embodiments, n is 2. In some embodiments, n is 1. In some embodiments, n is 0. In some embodiments, o is 5. In some embodiments, o is 4. In some embodiments, o is 3. In some embodiments, o is 2. In some embodiments, o is 1. In some embodiments, p is 3. In some embodiments, p is 2. In some embodiments, p is 1.

In some embodiments, each R^{a}, R^{b}, R^{c}, and R^{d} are independently selected from the group consisting of oxo, halogen, alkyl, heteroalkyl, cycloalkyl, heterocycloalkyl, alkoxy, amino (e.g., dihydroamino, alkylamino, or arylamino), hydroxy, or thiol, wherein the alkyl, heteroalkyl, cycloalkyl, alkoxy, amino (e.g., dihydroamino, alkylamino, or arylamino), hydroxy, thiol, or heterocycloalkyl is optionally substituted. In some embodiments, each R^{a} is independently selected from —OH, oxo, halogen, alkyl, or alkoxy, wherein the alkyl or alkoxy is optionally substituted. In some embodiments, each R^{b} is independently selected from —OH, oxo, halogen, or optionally substituted alkyl. In some embodiments, each R^{c} is independently selected from —OH, oxo, or optionally substituted alkyl. In some embodiments, each R^{d} is independently selected from —OH, oxo, alkyl (e.g., alkenyl or alkynyl), heteroalkyl, or each R^{d} is taken together to form an oxo, wherein the alkyl or heteroalkyl is optionally substituted. In some embodiments, the substituted alkyl of R^{d} is —COOH, —(C═O)alkyl, —(C═O)Oalkyl, —O(C═O)Oalkyl, —(C═O)S alkyl, wherein the alkyl is optionally substituted with —OH or halogen. In some embodiments, one R^{d} is taken together with another R^{d} to form a substituted or unsubstituted cycloalkyl or heterocycloalkyl.

In some embodiments, the alkyl of any one of R^{a}, R^{b}, R^{c}, or R^{d} is C₁-C₃ alkyl. In some embodiments, the alkyl of any one of R^{a}, R^{b}, R^{c}, or R^{d} is substituted with oxo and further optionally substituted with alkyl, hydroxy, halogen, heteroalkyl, alkoxy, thioether, wherein the alkyl, alkoxy, thioether, or heteroalkyl is further optionally substituted. In some embodiments, the alkoxy of any one of R^{a}, R^{b}, R^{c}, or R^{d} is C₁-C₃ alkoxy.

In some embodiments, Ring A is aromatic. In some embodiments, Ring A comprises at least one double bond. In some embodiments, Ring A comprises one double bond. In some embodiments, Ring A comprises two double bonds. In some embodiments, Ring B comprises at least one double bond. In some embodiments, Ring B comprises one double bond. In some embodiments, Ring C comprises one double bond. In some embodiments, Ring D comprises one double bond. In some embodiments, Ring A comprises at least one double bond and each of Ring B, Ring C, and Ring D consist of single bonds. In some embodiments, Ring A is aromatic and each of Ring B, Ring C, and Ring D consist of single bonds. In some embodiments, Ring A comprises at least one double bond and at least one of Ring B, Ring C, or Ring D comprises a double bond. In some embodiments, Ring A is aromatic and at least one of Ring B, Ring C, or Ring D comprises a double bond. In some embodiments, Ring A comprises at least one double bond and Ring B comprises a double bond. In some embodiments, Ring A comprises at least one double bond and Ring C comprises a double bond. In some embodiments, Ring A comprises at least one double bond and Ring D comprises a double bond.

In some embodiments, provided herein is a compound comprising a steroid radical and a statin radical, the steroid radical comprising a structure of Formula (V′):

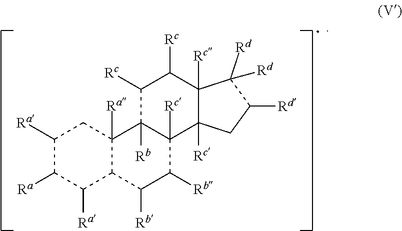

In some embodiments, [symbol] is a single bond or a double bond. In some embodiments, R^{a} [symbol] is hydrogen, —OH, or oxo. In some embodiments, each R^{a′} is independently selected from hydrogen, —OH, halogen, C₁-C₃ alkyl, and alkoxy. In some embodiments, R^{a″} is absent, hydrogen, or C₁-C₃ alkyl. In some embodiments, R^{b} is absent, hydrogen, halogen, or C₁-C₃ alkyl. In some embodiments, R^{b′} is hydrogen, halogen, —OH, oxo, or C₁-C₃ alkyl. In some embodiments, R^{b″} is hydrogen or —OH. In some embodiments, each R^{c} is independently hydrogen, —OH, oxo, or C₁-C₃ alkyl. In some embodiments, each R^{c′} is independently hydrogen or C₁-C₃ alkyl. In some embodiments, R^{c″} is hydrogen, —OH, C₁-C₃ alkyl, or —C(═O)H. In some embodiments, each R^{d} is independently hydrogen, —OH, —COOH, alkyl (e.g., alkylene, alkenyl, or alkynyl), heteroalkyl, or each R^{d} is taken together to form an oxo, wherein the alkyl or heteroalkyl is optionally substituted. In some embodiments, R^{d′} is hydrogen, —OH, C₁-C₃ alkyl (e.g., alkylene or alkenyl), or heteroalkyl. In some embodiments, one R^{d} is taken together with R^{d′} to form a substituted or unsubstituted cycloalkyl or heterocycloalkyl. In some embodiments, the statin radical is a radical of an active form of a statin described herein, such as a form of rosuvastatin described herein. Also provided in certain embodiments herein are pharmaceutical salts or solvates of a compound of Formula (V′).

In some embodiments, the structure of any one of Formula (V) or Formula (V′) consists of single bonds. In some embodiments, the structure of any one of Formula (V) or Formula (V′) comprises at least one double bond. In some embodiments, the structure of any one of Formula (V) or Formula (V′) comprises one double bond. In some embodiments, the structure of any one of Formula (V) or Formula (V′) comprises two double bonds. In some embodiments, the structure of any one of Formula (V) or Formula (V′) comprises three double bonds. In some embodiments, the structure of any one of Formula (V) or Formula (V′) comprises at least one aromatic ring. In some embodiments, the structure of any one of Formula (V) or Formula (V′) comprises one aromatic ring.

In some embodiments, R^{a} is —OH. In some embodiments, R^{a} is —OH and attached to a fully saturated cycloalkyl. In some embodiments, R^{a} is —OH and attached to an aryl. In some embodiments, R^{a} is oxo. In some embodiments, R^{a} is oxo and is adjacent to at least one double bond. In some embodiments, R^{a} is oxo and is adjacent to one double bond. In some embodiments, R^{a} is oxo and is adjacent to two double bonds.

In some embodiments, each R^{a′} is independently hydrogen or halogen (e.g., fluoro or chloro). In some embodiments, each R^{a′} is independently hydrogen or C₁-C₃ alkyl. In some embodiments, each R^{a′} is independently hydrogen or C₁-C₃ alkoxy. In some embodiments, each R^{a′} is attached to a single bond. In some embodiments, each R^{a′} is hydrogen. In some embodiments, at least one R^{a′} is attached to a double bond. In some embodiments, one R^{a′} is attached to a double bond. In some embodiments, each R^{a′} is attached to a double bond. In some embodiments, each R^{a′} is independently hydrogen or C₁-C₃ alkyl and attached to a single bond. In some embodiments, each R^{a′} is independently hydrogen or halogen, and one R^{a′} is attached to a double bond. In some embodiments, each R^{a′} is hydrogen and attached to a single bond. In some embodiments, each R^{a′} is hydrogen and attached to a double bond. In some embodiments, each R^{a′} is attached to an aryl and independently hydrogen or C₁-C₃ alkoxy. In some embodiments, each R^{a′} is hydrogen and attached to an aryl.

In some embodiments, R^{a″} is absent. In some embodiments, R^{a″} is hydrogen. In some embodiments, R^{a″} is C₁-C₃ alkyl. In some embodiments, R^{a} is —OH or oxo, each R^{a′}, is independently hydrogen or C₁-C₃ alkyl, and R^{a″} is C₁-C₃ alkyl. In some embodiments, R^{a} is —OH or oxo, each R^{a′} is independently hydrogen or C₁-C₃ alkoxy, and R^{a″} is C₁-C₃ alkyl. In some embodiments, R^{a} is oxo, each R^{a′} is independently hydrogen or halogen (e.g., fluoro or chloro), and R^{a″} is C₁-C₃ alkyl (e.g., methyl). In some embodiments, R^{a} is oxo, each R^{a′} is independently hydrogen or C₁-C₃ alkyl (e.g., methyl), and R^{a″} is C₁-C₃ alkyl (e.g., methyl). In some embodiments, R^{a} is oxo, each R^{a′} is hydrogen, and R^{a″} is C₁-C₃ alkyl (e.g., methyl). In some embodiments, R^{a} is —OH, each R^{a′} is independently hydrogen or C₁-C₃ alkoxy (e.g., methoxy), and R^{a″} is absent. In some embodiments, R^{a} is —OH, each R^{a′} is hydrogen, and R^{a″} is C₁-C₃ alkyl (e.g., methyl). In some embodiments, R^{a} is —OH, each R^{a′} is hydrogen, and R^{a″} is absent.

In some embodiments, R^{b} is absent. In some embodiments, R^{b} is hydrogen. In some embodiments, R^{b} is halogen (e.g., fluoro or chloro). In some embodiments, R^{b′} is hydrogen. In some embodiments, R^{b′} is halogen (e.g., fluoro or chloro). In some embodiments, R^{b′} is —OH. In some embodiments, R^{b′} is -oxo. In some embodiments, R^{b′} is C₁-C₃ alkyl (e.g., methyl). In some embodiments, R^{b′} is hydrogen, halogen (e.g., fluoro or chloro), or C₁-C₃ alkyl (e.g., methyl) and attached to a single bond. In some embodiments, R^{b′} is hydrogen or C₁-C₃ alkyl (e.g., methyl) and attached to a double bond. In some embodiments, R^{b″} is hydrogen. In some embodiments, R^{b}″ is —OH.

In some embodiments, R^{b} is hydrogen or halogen (e.g., fluoro or chloro), R^{b′} is hydrogen, halogen (e.g., fluoro or chloro), or C₁-C₃ alkyl (e.g., methyl), and R^{b″} is hydrogen. In some embodiments, R^{b} is hydrogen or halogen (e.g., fluoro or chloro), R^{b′} is hydrogen or halogen (e.g., fluoro or chloro), and R^{b″} is hydrogen. In some embodiments, R^{b} is halogen (e.g., fluoro or chloro), R^{b′} is halogen (e.g., fluoro or chloro), and R^{b″} is hydrogen. In some embodiments, R^{b} is halogen (e.g., fluoro or chloro), R^{b′} is hydrogen, and R^{b″} is hydrogen. In some embodiments, R^{b} is hydrogen, R^{b′} is halogen (e.g., fluoro or chloro), and R^{b″} is hydrogen. In some embodiments, R^{b} is hydrogen, R^{b′} is C₁-C₃ alkyl (e.g., methyl), and R^{b″} is hydrogen. In some embodiments, R^{b} is hydrogen, R^{b′} is hydrogen, and R^{b″} is —OH. In some embodiments, R^{b} is hydrogen, R^{b′} is oxo, and R^{b″} is hydrogen. In some embodiments, R^{b}, R^{b′}, and R^{b″} are each hydrogen.

In some embodiments, each R^{C} is independently hydrogen or —OH. In some embodiments, each R^{c} is independently hydrogen or oxo. In some embodiments, each R^{c} is hydrogen. In some embodiments, each R^{c′} is hydrogen. In some embodiments, each R^{c′} is C₁-C₃ alkyl. In some embodiments, R^{c″} is hydrogen. In some embodiments, R^{c″} is C₁-C₃ alkyl. In some embodiments, R″ is —C(═O)H.

In some embodiments, each R^{c} is hydrogen, each R^{c′} is hydrogen, and R^{c″} is C₁-C₃ alkyl. In some embodiments, each R^{x} is independently hydrogen or —OH, each R^{c′} is hydrogen, and R^{c″} is C₁-C₃ alkyl. In some embodiments, each R^{c} is independently hydrogen or oxo, each R^{c′} is hydrogen, and R^{c″} is C₁-C₃ alkyl. In some embodiments, each R^{c} is independently hydrogen or —OH, each R^{c′} is hydrogen, and R^{c″} is —C(═O)H. In some embodiments, each R^{c} is independently hydrogen or —OH, each R^{c′} is C₁-C₃ alkyl, and R^{c″} is hydrogen.

In some embodiments, one R^{d} is absent and R^{d′} and the other R^{d} are attached to a double bond. In some embodiments, the R^{d′} and the other R^{d} attached to a double bond are each hydrogen.

In some embodiments, one R^{d} is hydrogen and the other R^{d} is —OH, —COOH, alkyl (e.g., alkylene, alkenyl, or alkynyl), heteroalkyl, wherein the alkyl or heteroalkyl is optionally substituted. In some embodiments, one R^{d} is alkyl and the other R^{d} is —OH, —COOH, alkyl (e.g., alkylene, alkenyl, or alkynyl), heteroalkyl, wherein the alkyl or heteroalkyl is optionally substituted. In some embodiments, one R^{d} is optionally substituted alkoxy and the other R^{d} is —OH, —COOH, alkyl (e.g., alkylene, alkenyl, or alkynyl), heteroalkyl, wherein the alkyl or heteroalkyl is optionally substituted. In some embodiments, one R^{d} is —OH and the other R^{d} is —COOH, alkyl (e.g., alkylene, alkenyl, or alkynyl), heteroalkyl, wherein the alkyl or heteroalkyl is optionally substituted. In some embodiments, each R^{d} is independently hydrogen or —OH. In some embodiments, each R^{d} is independently optionally substituted alkyl or —OH. In some embodiments, each R^{d} is independently —COOH or —OH. In some embodiments, each R^{d} is independently —COOH or optionally substituted alkoxy. In some embodiments, each R^{d} is taken together to form an oxo. In some embodiments, each R^{d} is taken together to form an optionally substituted alkenyl. In some embodiments, the alkenyl is substituted with —COOH and alkyl. In some embodiments, the alkyl comprises saturated and unsaturated carbon bonds. In some embodiments, each R^{d} is independently optionally substituted alkyl or hydrogen. In some embodiments, the alkyl consists of saturated carbon bonds. In some embodiments, the alkyl is substituted with C₁-C₃ alkyl and alkyl further substituted with —COOH. In some embodiments, the alkyl is substituted with C₁-C₃ alkyl and alkyl further substituted with —OH.

In some embodiments, R^{d′} is hydrogen. In some embodiments, R^{d′} is —OH. In some embodiments, R^{d′} is C₁-C₃ alkyl (e.g., alkylene or alkenyl). In some embodiments, the C₁-C₃ alkyl is methyl. In some embodiments, the C₁-C₃ alkyl is CHCH. In some embodiments, R^{d′} is heteroalkyl. In some embodiments, the heteroalkyl is —O(C═O)C₁-C₃ alkyl.

In some embodiments, one R^{d} is taken together with R^{d′} to form an optionally substituted cycloalkyl or optionally substituted heterocycloalkyl. In some embodiments, one R^{d} is taken together with R^{d′} to form a heterocycloalkyl substituted with one or more alkyl groups. In some embodiments, one R^{d} is optionally substituted alkyl and the other R^{d} is taken together with R^{d′} to form a heterocycloalkyl substituted with one or more alkyl groups. In some embodiments, the alkyl is substituted with oxo and —OH. In some embodiments, the alkyl is substituted with oxo and alkyl further substituted with halogen (e.g., fluoro or chloro). In some embodiments, the heterocycloalkyl is an optionally substituted dioxolane. In some embodiments, the optionally substituted dioxolane is 2,2-dimethyl-1,3-dioxolane. In some embodiments, the optionally substituted dioxolane is 1,4-dioxaspiro[4.4]nonane.

In some embodiments, each R^{d} is independently hydrogen or optionally substituted alkyl and R^{d′} is hydrogen. In some embodiments, each R^{d} is independently hydrogen or optionally substituted alkyl and R^{d′} is C₁-C₃ alkyl. In some embodiments, each R^{d} is independently optionally substituted alkyl and R^{d′} is hydrogen. In some embodiments, each R^{d} is independently —OH or optionally substituted alkyl and R^{d′} is hydrogen. In some embodiments, each R^{d} is independently —COOH or optionally substituted alkoxy and R^{d′} is hydrogen. In some embodiments, each R^{d} is independently —OH or optionally substituted alkyl and R^{d′} is C₁-C₃ alkyl. In some embodiments, each R^{d} is independently —OH or optionally substituted alkyl and R^{d′} is —OH. In some embodiments, each R^{d} is independently —OH or optionally substituted alkyl and R^{d′} is alkyl (e.g., alkenyl). In some embodiments, each R^{d} is independently hydrogen or —OH and R^{d′} is hydrogen.

In some embodiments, each R^{d} is independently —OH or —COOH and R^{d′} is hydrogen. In some embodiments, each R^{d} and R^{d′} are hydrogen. In some embodiments, each R^{d} is optionally substituted alkenyl and R^{d′} is optionally substituted alkoxy. In some embodiments, each R^{d} is taken together to form an oxo and R^{d′} is hydrogen. In some embodiments, one R^{d} is optionally substituted alkyl and the other R^{d} is taken together with R^{d′} is to form an optionally substituted heterocycloalkyl.

In some embodiments, the alkyl or heteroalkyl of R^{d} or R^{d′} is substituted with one or more of the group consisting of —SH, —OH, —COOH, oxo, halogen, amino (e.g., dihydroamino, alkylamino, or arylamino), alkyl (e.g., alkenyl, alkynyl), heteroalkyl, ester, amide, sulfonic acid, and sulfone. In some embodiments, one R^{d} is taken together with R^{d′} to form substituted heterocycloalkyl.

In some embodiments, the alkyl of R^{d} is substituted with oxo and alkyl further substituted with hydroxyl. In some embodiments, the alkyl of R^{d} is substituted with oxo and alkyl further substituted with halogen (e.g., fluorine or chlorine). In some embodiments, the alkyl of R^{d} is substituted with oxo and C₁-C₃ alkyl. In some embodiments, the alkyl of R^{d} is substituted with oxo and alkyl further substituted with alkoxy further substituted with oxo and C₁-C₃ alkyl. In some embodiments, the alkyl of R^{d} is substituted with alkyl and alkyl further substituted with oxo and amino further substituted with alkyl further substituted with sulfonic acid. In some embodiments, the alkyl of R^{d} is substituted with oxo and thiol (e.g., thioether) further substituted with C₁-C₃ alkyl further substituted with halogen (e.g., fluorine or chlorine). In some embodiments, the alkyl of R^{d} is substituted with —OH. In some embodiments, the alkyl of R^{d} is substituted with oxo and hydroxyl (e.g., ether) further substituted with C₁-C₃ alkyl further substituted with halogen (e.g., fluorine or chlorine). In some embodiments, the alkoxy of R^{d} is substituted with oxo and alkoxy further substituted with alkyl.

In some embodiments, the C₁-C₃ alkyl is methyl, ethyl, propyl, isopropyl, butyl, or tert-butyl. In some embodiments, the C₁-C₃ alkyl is methyl. In some embodiments, the C₁-C₃ alkoxy is methoxy, ethyoxy, propyoxy, or isopropoxy. In some embodiments, the C₁-C₃ alkyl is methoxy.

In some embodiments, the steroid radical and the statin (e.g., rosuvastatin) radical described herein are joined by a linker (e.g., a bond). In some embodiments, the steroid radical described herein is joined to the statin (e.g., rosuvastatin) radical described herein through any one of R^{a}, R^{b}, R^{c}, or R^{d} of the steroid radical. In some embodiments, the steroid radical described herein is joined to the statin (e.g., rosuvastatin) radical described herein through any one of R^{a}, R^{b}, R^{c}, or R^{d}, and the R^{a}, R^{b}, R^{c}, or R^{d} through which the steroid radical is joined to the statin (e.g., rosuvastatin) radical comprises a hydroxyl radical (e.g., when together with the linker or second radical (where the linker is a bond), forms an ether), a thiol radical (e.g., when together with the linker or statin (e.g., rosuvastatin) radical (where the linker is a bond), forms a thioether), or a carboxylate radical (e.g., when taken together with the linker or second radical (where the linker is a bond), forms an ester or carbonate). In some embodiments, the connection between the thiol radical forms a thioester, a disulfide, or a thiocarbonate. In some embodiments, the connection between the carboxylate radical forms an anhydride. In some embodiments, the steroid radical is joined to the statin (e.g., rosuvastatin) radical through any one of R^{a}, R^{b}, R^{c}, or R^{d}, and the R^{a}, R^{b}, R^{c}, or R^{d} through which the statin (e.g., rosuvastatin) radical is joined to the statin (e.g., rosuvastatin) radical comprises an amino radical (e.g., when together with the linker or statin (e.g., rosuvastatin) radical (where the linker is a bond), forms an amide, carbamate, or thiocarbamate).

In some embodiments, the R^{a}, R^{b}, R^{c}, or R^{d} through which the steroid radical is joined to the statin (e.g., rosuvastatin) radical comprises a hydroxyl radical which together with the linker or with the statin (e.g., rosuvastatin) radical forms an ether. In some embodiments, the R^{a}, R^{b}, R^{C}, or R^{d} through which the steroid radical is joined to the statin (e.g., rosuvastatin) radical comprises a thiol radical which together with the linker or the statin (e.g., rosuvastatin) radical forms a thioether. In some embodiments, the R^{a}, R^{b}, R^{c}, or R^{d} through which the steroid radical is joined to the statin (e.g., rosuvastatin) radical comprises a carboxylate radical which together with the linker or the statin (e.g., rosuvastatin) radical forms an ester or a carbonate.

In some embodiments, the steroid radical has a structure of Formula (V) or Formula (V′) and the statin (e.g., rosuvastatin) radical does not have a structure of Formula (V) or Formula (V′). In some embodiments, the structure of Formula (V) or Formula (V′) has a melt and/or glass transition temperature at a temperature of at least 20° C. (e.g., at least 25° C., at least 30° C., at least 37° C., at least 40° C., at least 50° C., at least 100° C., or more) in its free form.

In some embodiments, the steroid radical is a solid (e.g., having a melting point of at least 30° C.) in its free form. In some embodiments, the statin (e.g., rosuvastatin) radical is a solid (e.g., having a melting point of at least 30° C.) in its free form. In some embodiments, the steroid radical is a corticosteroid (e.g., glucocorticoid or mineralcorticoid), a sex steroid, a neurosteroid, an aminosteroid, or a secosteroid.

In certain embodiments, provided herein is a compound comprising a steroid radical and a statin (e.g., rosuvastatin) radical, wherein the steroid radical has a structure of any one of Formula (V), Formula (V′), Formula C1, Formula 1C, Formula C2, Formula 2C, or Table 1 and the statin (e.g., rosuvastatin) radical has a structure of any one of Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2. In some embodiments, the steroid radical (e.g., having a structure of any one of Formula (V), Formula (V′), Formula C1, Formula 1C, Formula C2, Formula 2C, or Table 1) and the statin (e.g., rosuvastatin) radical (e.g., Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2) are joined by a linker (e.g., hydrolyzable linker). In some embodiments, the linker is a bond.

In certain embodiments, provided herein is a compound comprising a steroid. In some embodiments, provided herein is a compound comprising a statin (e.g., rosuvastatin). In some embodiments, provided herein is a compound comprising a linker (e.g., hydrolyzable linker). In some embodiments, the linker adjoins (e.g., covalently) the steroid and the statin (e.g., rosuvastatin). Also provided in certain embodiments herein are pharmaceutical salts or solvates of a compound.

In some embodiments, a compound provided herein comprises a steroid attached to a statin (e.g., rosuvastatin) through an optional linker (as such, forming a heteroalkyl bond (e.g., an ester, a carbonate, etc.), such as, whereby upon cleavage (e.g., hydrolysis) of the heteroalkyl bond, the steroid and/or statin (e.g., rosuvastatin) are released in their free form. In some embodiments, a steroid radical provided herein (e.g., a hydroxyl radical (e.g., anecortave desacetate radical)) is attached to an optional linker or a statin radical) (e.g., a hydroxyl radical, a carboxylic radical, a carbonyl radical, etc.) provided herein to form a compound provided herein.

Provided in some embodiments herein is a compound having a structure represented by Formula IC:

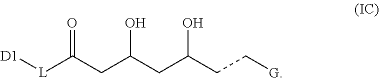

Provided in some embodiments herein is a compound having a structure represented by Formula IC′:

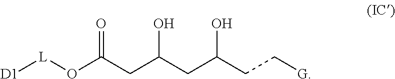

In some embodiments, [symbol] is a single bond or a double bond. In some embodiments, G is substituted heteroaryl or substituted carbocyclyl (e.g., as described elsewhere herein). In some embodiments, D1 is a radical of a steroid (e.g., a radical of a steroid described elsewhere herein).

In some embodiments, L is a bond or a linker (e.g., a linker described elsewhere herein). In some embodiments, the compound is a pharmaceutically acceptable salt or solvate.

In some embodiments, the hydroxyl groups of the structure represented by Formula IC or Formula IC′ are meso.

Provided in some embodiments herein is a compound having a structure represented by Formula IC1:

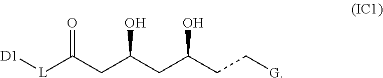

Provided in some embodiments herein is a compound having a structure represented by Formula IC1′:

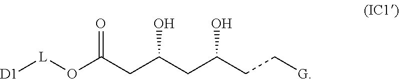

In some embodiments, G is substituted heteroaryl. In some embodiments, the substituted heteroaryl is selected from the group consisting of substituted pyrrolyl, substituted pyridinyl, substituted indolyl, substituted quinolinyl, and substituted pyrimidinyl.

In some embodiments, G is a substituted heteroaryl selected from the group consisting of substituted pyrrolyl, substituted pyridinyl, substituted indolyl, substituted quinolinyl, and substituted pyrimidinyl.

In some embodiments, G is heteroaryl substituted with one or more substituents, each substituent being independently selected from the group consisting of substituted or unsubstituted amide, substituted or unsubstituted alkyl, substituted or unsubstituted heteroalkyl, substituted or unsubstituted aryl, substituted or unsubstituted carbocyclyl, and substituted or unsubstituted sulfonamide.

In some embodiments, G is heteroaryl substituted with one or more substituents, each substituent being independently selected from the group consisting of substituted or unsubstituted alkyl (e.g., branched alkyl), substituted or unsubstituted heteroalkyl, substituted or unsubstituted aryl, substituted or unsubstituted carbocyclyl, and substituted or unsubstituted sulfonamide.

In some embodiments, G is heteroaryl substituted with substituted or unsubstituted alkyl (e.g., branched alkyl), substituted or unsubstituted heteroalkyl, and substituted or unsubstituted aryl.

In some embodiments, G is heteroaryl substituted with substituted or unsubstituted alkyl (e.g., branched alkyl) and substituted or unsubstituted aryl.

In some embodiments, G is heteroaryl substituted with substituted or unsubstituted carbocyclyl and substituted or unsubstituted aryl.

In some embodiments, G is heteroaryl substituted with branched alkyl, substituted or unsubstituted aryl, and substituted or unsubstituted sulfonamide. In some embodiments, G is heteroaryl substituted with branched alkyl (e.g., isopropyl), substituted aryl (e.g., phenyl substituted with fluoro), and sulfonamide substituted with alkyl (e.g., N-methylmethanesulfonamidyl). In some embodiments, G is heteroaryl substituted with branched alkyl (e.g., isopropyl), phenyl substituted with fluoro, and N-methylmethanesulfonamidyl.

In some embodiments, G is selected from the group consisting of substituted pyrrolyl, substituted pyridinyl, substituted indolyl, substituted quinolinyl, and substituted pyrimidinyl. In some embodiments, G is substituted pyrrolyl. In some embodiments, G is substituted pyridinyl.

In some embodiments, G is substituted indolyl. In some embodiments, G is substituted quinolinyl. In some embodiments, G is substituted pyrimidinyl.

In some embodiments, G is pyrinidinyl with branched alkyl, substituted or unsubstituted aryl, and substituted or unsubstituted sulfonamide. In some embodiments, G is pyrinidinyl substituted with branched alkyl (e.g., isopropyl), substituted aryl (e.g., phenyl substituted with fluoro), and sulfonamide substituted with alkyl (e.g., N-methylmethanesulfonamidyl). In some embodiments, G is pyrinidinyl substituted with branched alkyl (e.g., isopropyl), phenyl substituted with fluoro, and N-methylmethanesulfonamidyl.

In some embodiments, G is pyridinyl substituted with branched alkyl (e.g., isopropyl), phenyl substituted with fluoro, and unsubstituted heteroalkyl (e.g., CH₂OCH3).

In some embodiments, G is pyridinyl substituted with branched alkyl (e.g., isopropyl) and phenyl substituted with fluoro.

In some embodiments, G is quinolinyl substituted with cyclopropyl and phenyl substituted with fluoro.

In some embodiments, G is substituted carbocyclyl.

In some embodiments, a compound described herein has a structure represented by Formula D:

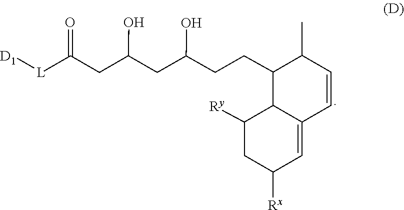

In some embodiments, a compound described herein has a structure represented by Formula D′:

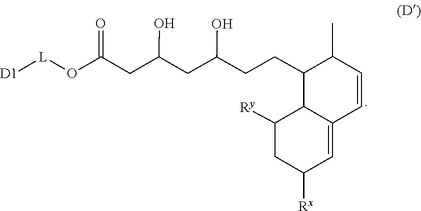

In some embodiments, [symbol] is a single bond or a double bond. In some embodiments, R^{x} is hydrogen, alkyl, or hydroxyl (e.g., as described elsewhere herein). In some embodiments, R^{y} is substituted alkoxy (e.g., as described elsewhere herein). In some embodiments, D1 is a radical of a steroid (e.g., a radical of a steroid described elsewhere herein). In some embodiments, L is a bond or a linker (e.g., a linker described elsewhere herein). In some embodiments, the compound is a pharmaceutically acceptable salt or solvate.

In some embodiments, the hydroxyl groups of the structure represented by Formula D or Formula D′ are meso.

Provided in some embodiments herein is a compound having a structure represented by Formula IIC:

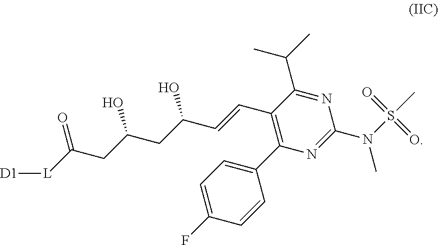

Provided in some embodiments herein is a compound having a structure represented by Formula IIC′:

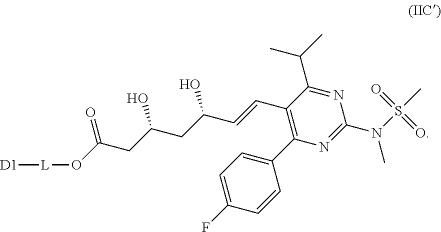

In some embodiments, D1 has a structure represented by Formula C1:

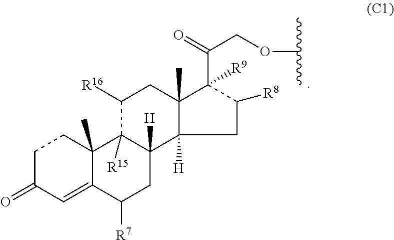

In some embodiments, D1 has a structure represented by Formula 1C:

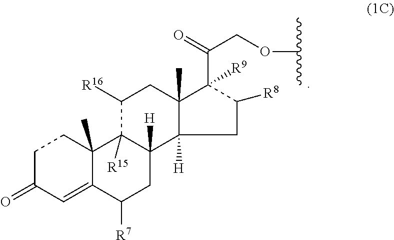

In some embodiments, [symbol] is a single bond or a double bond. In some embodiments, R⁷ is hydrogen or halogen. In some embodiments, R⁸ is hydrogen or C₁-C₄ alkyl. In some embodiments, R⁹ is absent, hydrogen, or hydroxyl. In some embodiments, R¹⁵ is absent, hydrogen, or halogen. In some embodiments, R¹⁶ is hydrogen or hydroxyl. In some embodiments, R⁸, R⁹, R¹⁵, and R¹⁶ are described elsewhere herein. In some embodiments, the compound is a pharmaceutically acceptable salt or solvate.

In some embodiments, D1 has a structure represented by Formula C2:

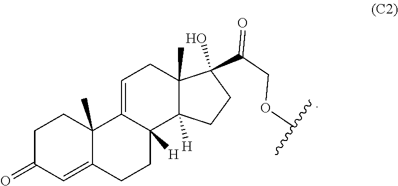 [SMILES: *OCC(=O)[C@@]1(O)CC[C@H]2[C@@H]3CCC4=CC(=O)CC[C@]4(C)C3=CC[C@@]21C]

In some embodiments, D1 has a structure represented by Formula 2C:

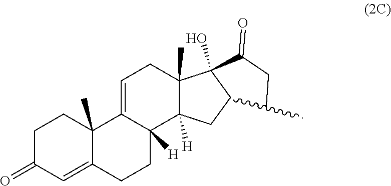 [SMILES: *CC(=O)[C@@]1(O)CC[C@H]2[C@@H]3CCC4=CC(=O)CC[C@]4(C)C3=CC[C@@]21C]

Provided in some embodiments herein is a compound having a structure represented by Formula IA:

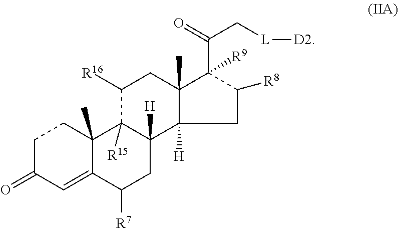

Provided in some embodiments herein is a compound having a structure represented by Formula IA′:

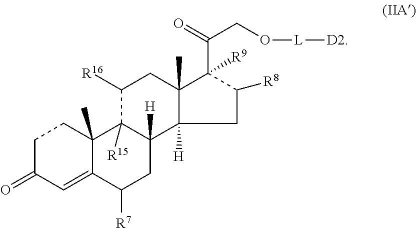

In some embodiments, [symbol] is a single bond or a double bond. In some embodiments, R⁷ is hydrogen or halogen. In some embodiments, R⁸ is hydrogen or C₁-C₄ alkyl. In some embodiments, R⁹ is absent, hydrogen, or hydroxyl. In some embodiments, R¹⁵ is absent, hydrogen, or halogen. In some embodiments, R¹⁶ is hydrogen or hydroxyl. In some embodiments, D2 is a radical of a statin (or an active form thereof), such as any statin or active form of a statin described herein (e.g., rosuvastatin). In some embodiments, L is a bond or a linker, such as any linker described herein. In some embodiments, L is a bond. In some embodiments, the compound having a structure of Formula IA or Formula IA′ is a pharmaceutically acceptable salt or solvate of the compound.

In some embodiments, R⁷ is hydrogen.

In some embodiments, R⁸ is methyl.

In some embodiments, R⁸ is hydrogen.

In some embodiments, R⁹ is hydroxyl.

In some embodiments, R¹⁵ is fluoro.

In some embodiments, R¹⁵ is absent.

In some embodiments, R¹⁵ is hydrogen.

In some embodiments, R¹⁶ is hydroxyl.

In some embodiments, R¹⁶ is hydrogen.

In some embodiments, a compound provided herein has a structure represented by Formula IIB:

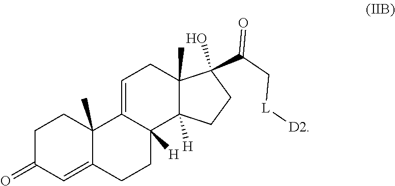

In some embodiments, a compound provided herein has a structure represented by Formula IIB′:

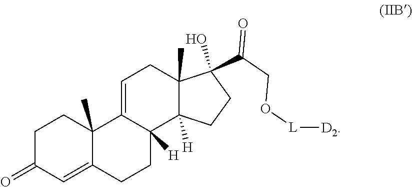

In some embodiments, D2 is a radical of a statin (or an active form thereof), such as any statin or active form of a statin described herein (e.g., rosuvastatin). In some embodiments, L is a bond or a linker, such as any linker described herein. In some embodiments, L is a bond. In some embodiments, the compound having a structure of Formula IB or Formula IB′ is a pharmaceutically acceptable salt or solvate of the compound.

In some embodiments, D2 is a radical of a statin or an active form of a statin (e.g., rosuvastatin). In some embodiments, D2 is a radical of a statin (e.g., rosuvastatin). In some embodiments, D2 is a radical of an active form of a statin (e.g., rosuvastatin). In some embodiments, D2 is a radical of atorvastatin, rosuvastatin, cerivastatin, fluvastatin, pitavastatin, lovastatin, mevastatin, simvastatin, pravastatin, or an active form thereof. In some embodiments, D2 is a radical of cerivastatin, fluvastatin, pitavastatin, lovastatin, mevastatin, simvastatin, pravastatin, or an active form thereof.

In some embodiments, D2 is a radical of atorvastatin.

In some embodiments, D2 is a radical of rosuvastatin.

In some embodiments, D2 is not a radical of simvastatin, atorvastatin, or rosuvastatin (or an active form thereof).

In some embodiments, D2 is not a radical of simvastatin (or an active form thereof).

In some embodiments, D2 is not a radical of atorvastatin (or an active form thereof).

In some embodiments, D2 is not a radical of rosuvastatin (or an active form thereof).

In some embodiments, D2 is a radical of a statin or an active form thereof, wherein the radical is on a carboxylate of the statin (e.g., rosuvastatin) or the active form thereof. In some embodiments, D2 is a radical of an active form of a statin (e.g., rosuvastatin), wherein the radical is on a carboxylate of the active form of the statin (e.g., rosuvastatin).

In some embodiments, D2 is a radical of rosuvastatin, wherein the radical resides on the carboxylate group of rosuvastatin.

In some embodiments, D2 is a radical of rosuvastatin, wherein the radical resides on a carbonyl carbon of rosuvastatin.

In some embodiments, D2 has a structure represented by Formula B1:

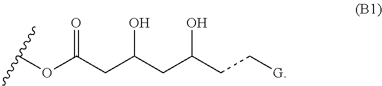

In some embodiments, D2 has a structure represented by Formula 1B:

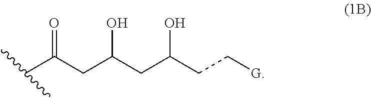

In some embodiments, [symbol] is a single bond or a double bond. In some embodiments, G is substituted heteroaryl or substituted carbocyclyl. In some embodiments, G is described elsewhere herein.

In some embodiments, the hydroxyl groups of the structure represented by Formula B1 or Formula 1B are meso.

In some embodiments, D2 has a structure represented by Formula B1′:

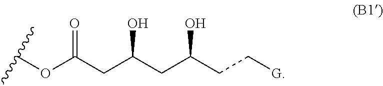

In some embodiments, D2 has a structure represented by Formula 1B′:

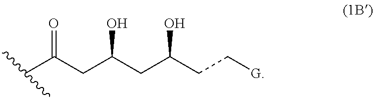

In some embodiments, D2 has a structure represented by Formula B1″:

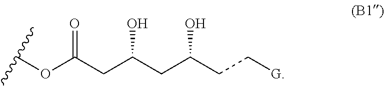

In some embodiments, D2 has a structure represented by Formula 1B″:

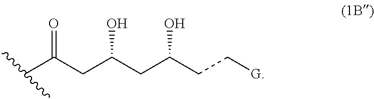

In some embodiments, G is described elsewhere herein. In some embodiments, G is substituted carbocyclyl (e.g., as described elsewhere herein). In some embodiments, G is substituted heteroaryl (e.g., as described elsewhere herein).

In some embodiments, D2 has a structure represented by Formula B2:

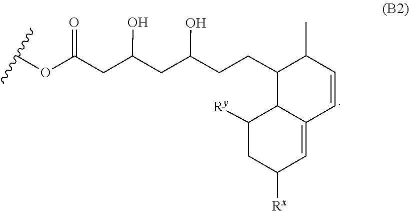

In some embodiments, D2 has a structure represented by Formula 2B:

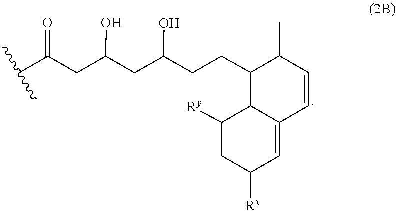

In some embodiments, the hydroxyl groups of the structure represented by Formula B2 or Formula 2B are meso.

In some embodiments, D2 has a structure represented by Formula B2′:

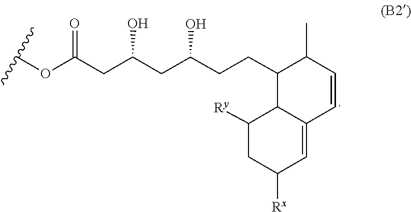

In some embodiments, D2 has a structure represented by Formula 2B′:

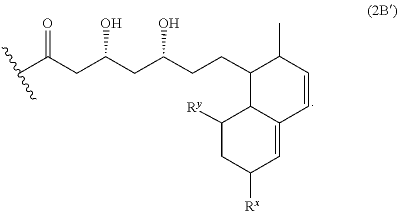

In some embodiments, D2 has a structure represented by Formula 2B″:

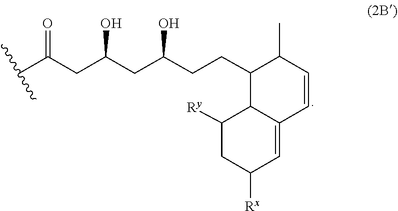

In some embodiments, R^{x} is hydrogen, alkyl, or hydroxyl. In some embodiments, R^{y} is substituted alkoxy.

In some embodiments, R^{x} is hydrogen.

In some embodiments, R^{x} is hydroxyl.

In some embodiments, R^{x} s alkyl. In some embodiments, R^{x} is methyl.

In some embodiments, R^{y} is branched alkoxy substituted with oxo. In some embodiments, R^{y} is —O(C═O)CH(CH₃)CH₂CH₃ or —O(C═O)C(CH₃)₂CH₂CH₃. In some embodiments, R^{y} is —O(C═O)CH(CH₃)CH₂CH₃. In some embodiments, R^{y} is —O(C═O)C(CH₃)₂CH₂CH₃.

In some embodiments, D2 is a carboxyl radical of an active form of lovastatin, a carboxyl radical of an active form of mevastatin, a carboxyl radical of an active form of simvastatin, or a carboxyl radical of pravastatin (e.g., such statins being shown in Table 2).

In some embodiments, D2 is a radical of rosuvastatin (or an active form thereof), such as any form of rosuvastatin described herein. In some embodiments, L is a bond or a linker, such as any linker described herein. In some embodiments, L is a bond. In some embodiments, the compound having a structure of Formula IIB or Formula IIB′ is a pharmaceutically acceptable salt or solvate of the compound.

In some embodiments, D2 is a radical of rosuvastatin, wherein the radical resides on the carboxylate group of rosuvastatin.

In some embodiments, D2 is a radical of rosuvastatin, wherein the radical resides on a carbonyl carbon of rosuvastatin.

In some embodiments, D2 has a structure represented by Formula A:

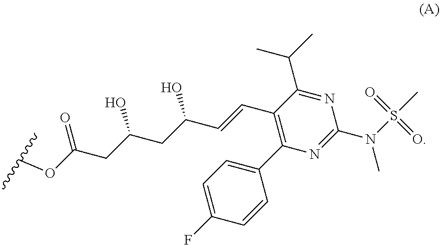 [SMILES: *OC(=O)C[C@H](O)C[C@H](O)/C=C/c1c(-c2ccc(F)cc2)nc(N(C)S(C)(=O)=O)nc1C(C)C]

In some embodiments, D2 has a structure represented by Formula B′:

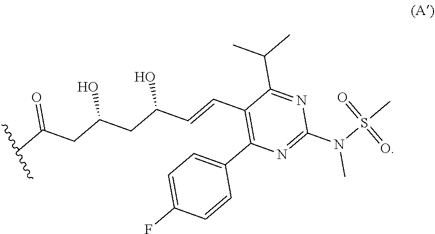 [SMILES: *C(=O)C[C@H](O)C[C@H](O)/C=C/c1c(-c2ccc(F)cc2)nc(N(C)S(C)(=O)=O)nc1C(C)C]

In some embodiments, either or both of D1 or D2 are attached to L through a hydroxyl radical of the D1 or D2. In some embodiments, D1 and D2 are attached to L through a hydroxyl radical of the D1 or D2. In some embodiments, D1 and D2 are attached to L through a hydroxyl radical of the D1 or D2.

In some embodiments, either or both of D1 or D2 are attached to L through a hydroxyl radical of the D1 or D2. In some embodiments, D1 and D2 are attached to L through a hydroxyl radical of the D1 or D2.

In some embodiments, D1 is a steroid (e.g., dexamethasone, anecortave (e.g., anecortave desacetate), etc.). In some embodiments, D1 is or is derived from anecortave (e.g., anecortave acetate or anecortave desacetate). In some embodiments, D1 is a steroid (e.g., dexamethasone, anecortave (e.g., anecortave desacetate), etc.). In some embodiments, the steroid is a corticosteroid (e.g., glucocorticoid or mineralcorticoid), a sex steroid, a neurosteroid, an aminosteroid, or a secosteroid. In some embodiments, D2 is not a steroid.

In some embodiments, the steroid is a glucocorticoid. In some embodiments, the glucocorticoid is selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, loprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol, or the like.

In some embodiments, the steroid is a mineralocorticoid. In some embodiments, the mineralocorticoid is selected from the group consisting of aldosterone, fludrocortisone, deoxycorticosterone, and corticosterone, or the like. In some embodiments, the mineralocorticoid is canrenone (e.g., potassium canrenoate), drospirenone, eplerenone, spirolactone, or a metabolite thereof (e.g., 7α-thiomethylspironolactone, canrenone, 6p-hydroxy-7α-thiomethylspironolactone, and 7α-thiospironolactone).

In some embodiments, the steroid is an anabolic steroid. In some embodiments, the anabolic steroid is selected from the group consisting of androisoxazole, androstenediol, bolandiol, bolasterone, clostebol, ethylestrenol, formyldienolone, 4-hydroxy-19-nortestosterone, methandriol, methenolone, methyltrienolone, nandrolone, norbolethone, oxymesterone, stenbolone, and trenbolone, or the like.

In some embodiments, the steroid is an androgenic steroid. In some embodiments, the androgenic steroid is selected from the group consisting of boldenone, fluoxymesterone, mestanolone, mesterolone, methandrostenolone, 17-methyltestosterone, 17-α-methyltestosterone 3-cyclopentyl enol ether, norethandrolone, normethandrone, oxandrolone, oxymesterone, oxymetholone, prasterone, stanlolone, stanozolol, testosterone, testosterone 17-chloral hemiacetal, testosterone proprionate, testosterone enanthate tiomesterone dehydroepiandrosterone (DHEA), androstenedione, androstenediol, androsterone, dihydrotestosterone (DHT), and androstanolone, or the like.

In some embodiments, the steroid is a progestin steroid. In some embodiments, the progestin steroid is selected from the group consisting of progesterone, norethisterone, norethisterone acetate, gestodene, levonorgestrel, allylestrenol, anagestone, desogestrel, dimethisterone, dydrogesterone, ethisterone, ethynodiol, ethynodiol diacetate, etonogestrel, gestodene, ethinylestradiol, haloprogesterone, 17-hydroxy-16-methylene-progesterone, 17-alpha-hydroxyprogesterone, lynestrenol, medroxyprogesterone, melengestrol, norethindrone, norethynodrel, norgesterone, gestonorone, norgestimate, norgestrel, levonorgestrel, norgestrienone, norvinisterone, pentagestrone, MENT (7-methyl-19-testosterone); norelgestromin, and trimigestone drospirenone, tibolone, and megestrol, or the like.

In some embodiments, the steroid is an estrogen steroid. In some embodiments, the estrogen steroid is selected from the group consisting of estradiol, estrone, eguilenin, equilin, estradiol benzoate, estriol, ethinyl estradiol, mestranol, moxestrol, mytatrienediol, quinestradiol, and quinestrol, or the like.

In some embodiments, the steroid is selected from the group consisting of abiraterone, cyproterone acetate, dutasteride, enzalutamide, finasteride, galeterone, fusidic acid, cholesterol, 11-deoxycortisol, 11-deoxycorticosterone, pregnenolone, cholic acid, chenodeoxycholic acid, ursodeoxycholic acid, obeticholic acid, tetrahydrocortisone, tetrahydrodeoxycortisol, tetrahydrocorticosterone, 5α-dihydrocorticosterone, 5α-dihydropregesterone, flugestone, prebediolone, chlormadinone acetate, medrogestone, and segesterone acetate, or the like.

In some embodiments, the steroid is an anti-angiogenic or an intraocular pressure (IOP) lowering steroid. In some embodiments, the angiostatic) lowering steroid is selected from the group consisting of anecortave acetate, anecortave (e.g., anecortave desacetate), 11-epicortisol, 17α-hydroxyprogesterone, tetrahydrocortexolone, and tetrahydrocortisol, or the like. In some embodiments, the angiostatic steroid is anecortave desacetate.

In some embodiments, the steroid is a cholic acid-related bile acid steroid. In some embodiments, the cholic acid-related bile acid steroid is selected from the group consisting of deoxycholic acid, apocholic acid, dehydrocholic acid, glycochenodeoxycholic acid, glycocholic acid, glycodeoxycholic acid, hyodeoxycholic acid, lithocholic acid, α-muricholic acid, β-muricholic acid, γ-muricholic acid, ω-muricholic acid, taurochenodeoxycholic acid, taurocholic acid, taurodeoxycholic acid, taurolithocholic acid, and tauroursodeoxycholic acid, or the like.

In some embodiments, the steroid is a neurosteroid. In some embodiments, the neurosteroid is selected from the group consisting of alphaxalone, alphadolone, hydroxydione, minaxolone, tetrahydrodeoxycorticosterone, allopregnanolone, pregnanolone, ganoxolone, 3α-androstanediol, epipregnanolone, isopregnanolone, and 24(S)-hydroxycholesterol, or the like.

In some embodiments, the steroid is a steroid pheromone. In some embodiments, the steroid pheromone is selected from the group consisting of androstadienol, androstadienone, androstenol, androstenone, estratetraenol, 5-dehydroprogesterone, 6-dehydro-retroprogesterone, allopregnanolone, and hydroxyprogesterone caproate, or the like.

In some embodiments, the steroid is a steroid metabolite. In some embodiments, the steroid metabolite is selected from the group consisting of tetrahydrotriamcinolone, cortienic acid, 11-dehydrocorticosterone, 11β-hydroxypregnenolone, ketoprogesterone, 17-hydroxypregnenolone, 17,21-dihydroxypregnenolone, 18-hydroxycorticosterone, deoxycortisone, 21-hydroxypregnenolone, and progesterone, or the like.

In some embodiments, the steroid is a progestin. In some embodiments, the progestin is selected from the group consisting of allopregnone-3α,20α-diol, allopregnone-3β,20β-diol, allopregnane-3β,21-diol-11,20-dione, allopregnane-3β,17α-diol-20-one, 3,20-allopregnanedione,3β,11β,17α,20β,21-pentol, allopregnane-3β,17α,20β,21-tetrol, allopregnane-3α,11β,17α,21-tetrol-20-one, allopregnane-3β,11β,17α,21-tetrol-20-one, allopregnane-3β,17α,20β-triol, allopregnane-3β,17α,21-triol-11,20-dione, allopregnane-3β,11β,21-triol-20-one, allopregnane-3β,17α,21-triol-20-one, allopregnane-3α-ol-20-one, allopregnane-3β-ol-20-one, pregnanediol, 3,20-pregnanedione, 4-pregnene-20,21-diol-3,11-dione, 4-pregnene-11β,17α,20β3,21-tetrol-3-one, 4-pregnene-17α,203,21-triol-3,11-dione, 4-pregnene-17α,203,21-triol-3-one, and pregnenolone, or the like.

In some embodiments, D1 is a (e.g., hydroxyl or carboxyl) radical of a compound selected from the group consisting of:

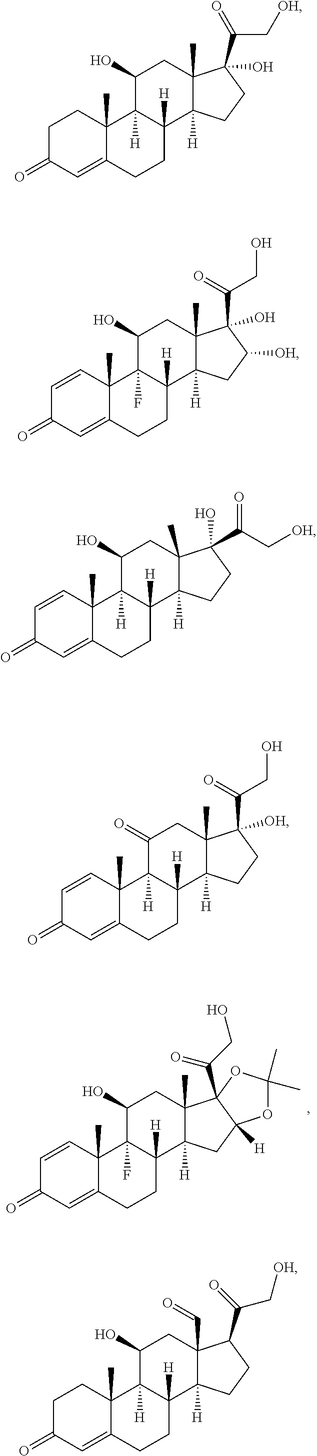

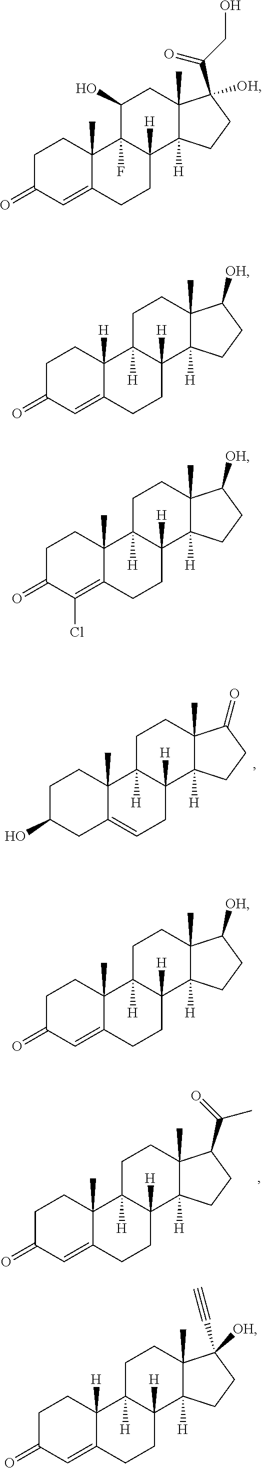

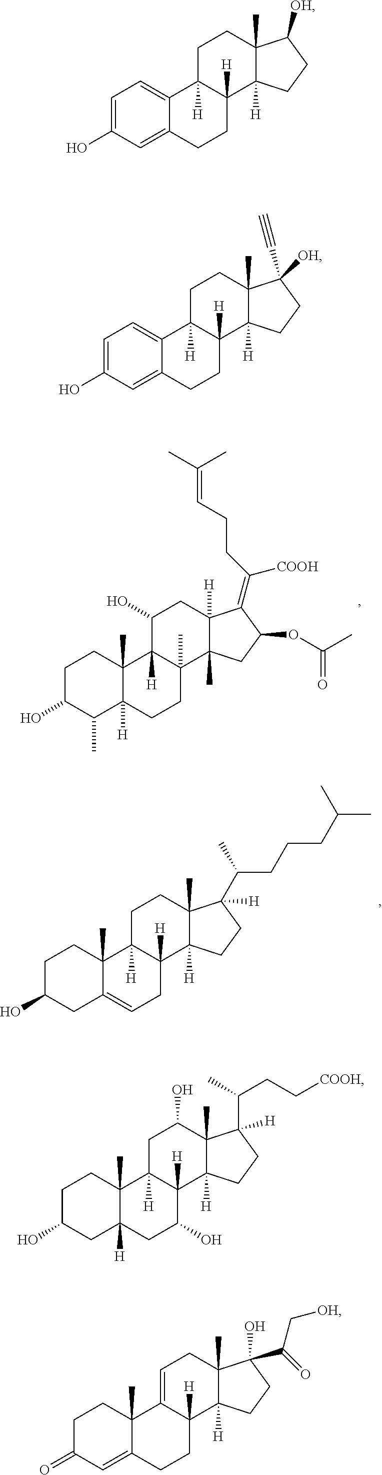

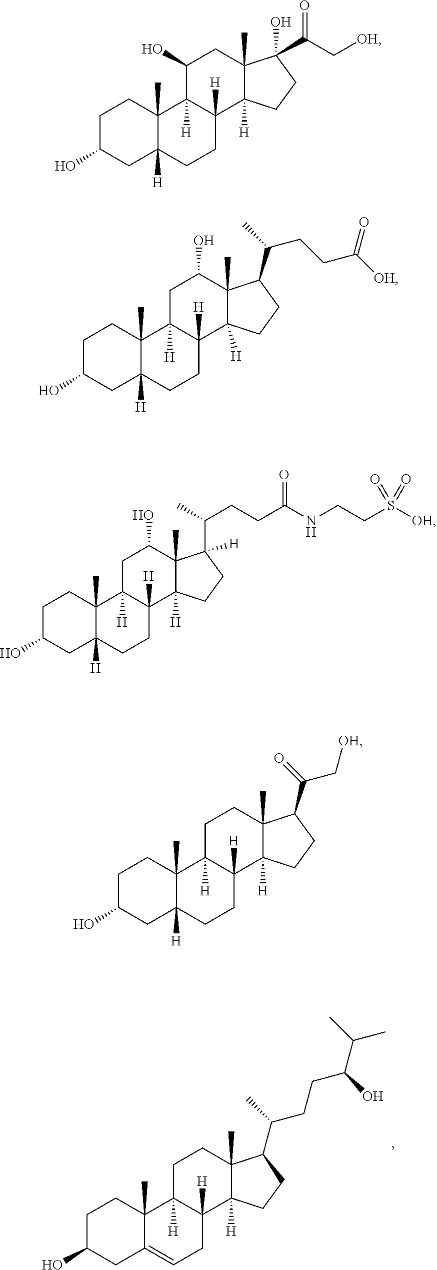

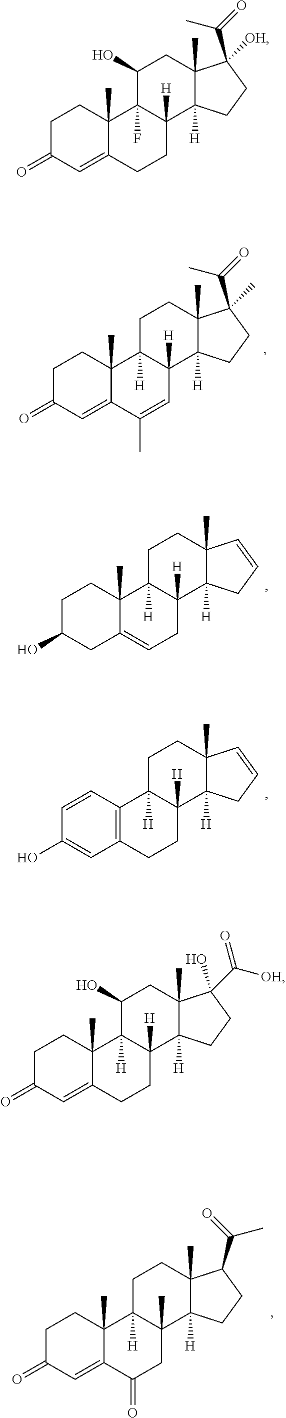

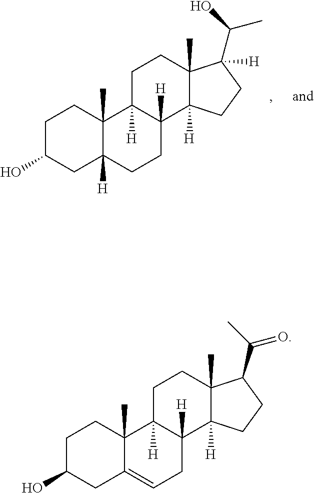

In some embodiments, the D1 is a (e.g., hydroxyl or carboxyl) radical of a compound selected from the group consisting of:

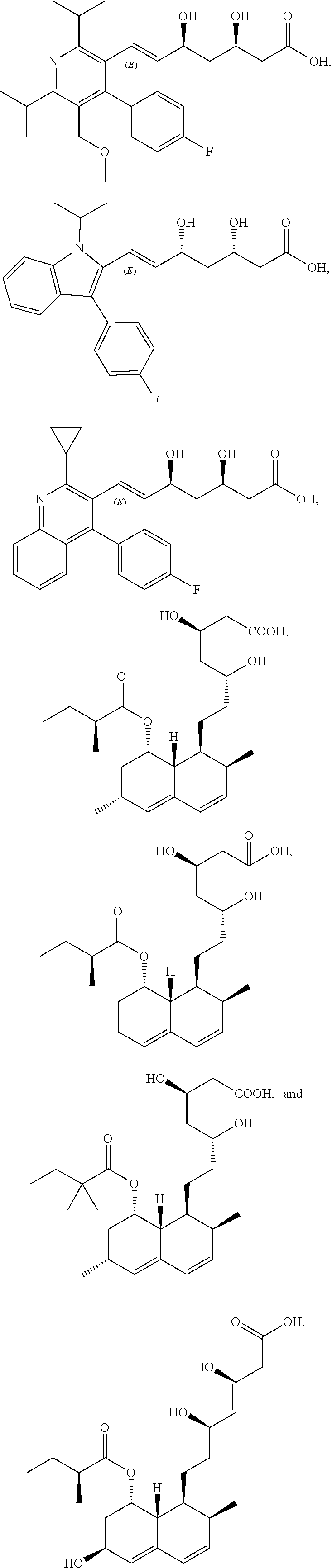

In some embodiments, the D2 is a (e.g., hydroxyl or carboxyl) radical of a compound selected from the group consisting of:

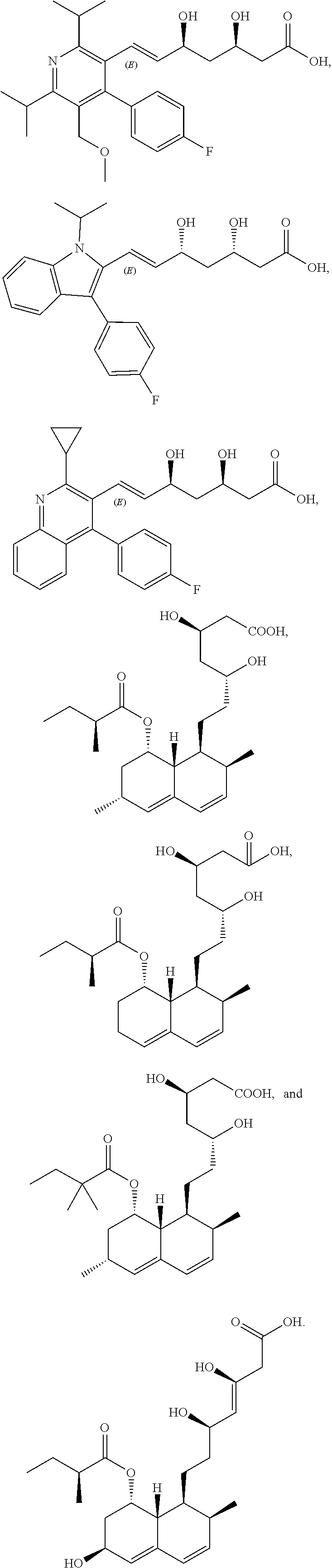

In some embodiments, D2 is a radical of a statin. In some embodiments, the statin is selected from the group consisting of atorvastatin, rosuvastatin, cerivastatin, fluvastatin, pitavastatin, lovastatin, mevastatin, simvastatin, and pravastatin, or a fragment, active form, or radical of any of the foregoing.

In some embodiments, D2 is a radical of a statin. In some embodiments, the statin is selected from the group consisting of cerivastatin, fluvastatin, pitavastatin, lovastatin, mevastatin, simvastatin, and pravastatin, or a fragment, active form, or radical of any of the foregoing.

In some embodiments, L (the linker) is a bond, substituted or unsubstituted alkyl, substituted or unsubstituted heteroalkyl, substituted or unsubstituted alkoxy, substituted or unsubstituted carbocyclyl, or substituted or unsubstituted heterocyclyl.

In some embodiments, L is a bond.

In some embodiments, L (the linker) is substituted or unsubstituted alkyl, substituted or unsubstituted heteroalkyl, substituted or unsubstituted alkoxy, substituted or unsubstituted carbocyclyl, or substituted or unsubstituted heterocyclyl.

In some embodiments, the linker is the linker (e.g., L) is or comprises -(Q¹-M-Q²)-. In some embodiments, Q¹ and Q² are each independently absent or (C═X¹)X². In some embodiments, X¹ is 0 or S. In some embodiments, X² is O, S, or NR¹. In some embodiments, R¹ is hydrogen or C₁-C₆ alkyl. In some embodiments, M comprises one or more linker group, each linker group being independently selected from the group consisting of substituted or unsubstituted alkyl (e.g., C₁-C₆ alkyl), substituted or unsubstituted aryl, substituted or unsubstituted heteroaryl, and substituted or unsubstituted heteroalkyl (e.g., C₁-C₆ heteroalkyl).

In some embodiments, Q¹ and Q² are each independently absent, C═O, C═S, (C═O)O, (C═O)S, (C═O)N, or (C═S)S. In some embodiments, Q¹ and Q² are each independently absent, C═O, or (C═O)O.

In some embodiments, Q¹ is absent, C═O, C═S, (C═O)O, (C═O)S, (C═O)N, or (C═S)S. In some embodiments, Q¹ is C═O or (C═O)O.

In some embodiments, Q¹ is absent.

In some embodiments, Q¹ is C═O.

In some embodiments, Q¹ is C═S.

In some embodiments, Q¹ is (C═O)O.

In some embodiments, Q¹ is (C═O)S.

In some embodiments, Q¹ is (C═O)N.

In some embodiments, Q¹ is (C═S)S.

In some embodiments, Q² is absent, C═O, C═S, (C═O)O, (C═O)S, (C═O)N, or (C═S)S. In some embodiments, Q² is C═O or (C═O)═O.

In some embodiments, Q² is absent.

In some embodiments, Q² is C═O.

In some embodiments, Q² is C═S.

In some embodiments, Q² is (C═)O.

In some embodiments, Q² is (C═O)S.

In some embodiments, Q² is (C═O)N.

In some embodiments, Q² is (C═S)S.

Unless stated specifically otherwise herein, each instance of (C═X¹)X² includes a specific and explicit recitation of (C═X¹)X² and X²(C═X¹). Likewise, unless stated specifically otherwise herein, each instance of (C═O)O includes a specific and explicit recitation of (C═O)O and O(C═O); each instance of (C═O)N includes a specific and explicit recitation of (C═O)N and N(C═O); each instance of (C═O)S includes a specific and explicit recitation of (C═O)S and S(C═O); and each instance of (C═S)S includes a specific and explicit recitation of (C═S)S and S(C═S). In some instances, X² (e.g., O, N, or S) taken together with any steroid, statin, or linker provided herein to form a C—X² bond (e.g., C—O, C—S, or C—N).

In some embodiments, M is substituted or unsubstituted alkyl (e.g., C₁-C₆ alkyl), substituted or unsubstituted heteroalkyl (e.g., C₁-C₆ heteroalkyl), or substituted or unsubstituted aryl.

In some embodiments, M is substituted or unsubstituted alkyl (e.g., C₁-C₆ alkyl). In some embodiments, M is substituted or unsubstituted C₁-C₆ alkyl. In some embodiments, M is substituted (e.g., C₁-C₆ alkyl) alkyl. In some embodiments, M is substituted alkyl (e.g., C₁-C₆ alkyl), the alkyl being substituted with one or more substituent, each substituent being independently selected from the group consisting of oxo, halo, alkyl, and heteroalkyl (e.g., —NHCOCH₃). In some embodiments, M is alkyl (e.g., C₁-C₆ alkyl) substituted with oxo. In some embodiments, M is unsubstituted alkyl (e.g., C₁-C₆ alkyl).

In some embodiments, M is substituted or unsubstituted heteroalkyl (e.g., C₁-C₆ heteroalkyl). In some embodiments, M is substituted or unsubstituted C₁-C₆ heteroalkyl. In some embodiments, M is unsubstituted heteroalkyl (e.g., C₁-C₆ heteroalkyl).

In some embodiments, M is substituted or unsubstituted aryl.

In some embodiments, M is alkyl (e.g., methyl) substituted with oxo and Q¹ and Q² are absent.

In some embodiments, Q¹ is C═O, Q² is absent, and M is —CH₂—, —CH₂CH₂—, —CHCH₃—, or —CH(NHCOCH₃)CH₂—.

In some embodiments, the linker (e.g., L) is a bond.

In some embodiments, a hydroxyl radical or a carboxylate radical of any one of Formula (V), Formula (V′), Formula C1, Formula 1C, Formula C2, Formula 2C, or Table 1 is attached to a hydroxyl radical or a carboxylate radical of another of any one of Formula A, Formula A′, Formula B1, Formula 1B, Formula B1′, Formula 1B′, Formula B1′″, Formula 1B′″, Formula B2, Formula 2B, Formula B2′, Formula 2B′, Formula B2″, Formula 2B′″, or a radical of a compound provided in Table 2 through a linker. In some embodiments, a hydroxyl radical of any one of Formula (V), Formula (V′), Formula C1, Formula 1C, Formula C2, Formula 2C, or Table 1 is attached to a hydroxyl radical of any one of Formula A, Formula A′, Formula B1, Formula 1B, Formula B1′, Formula 1B′, Formula B1″, Formula 1B′″, Formula B2, Formula 2B, Formula B2′, Formula 2B′, Formula B2″, Formula 2B″, or a radical of a compound provided in Table 2 through a linker. In some embodiments, a carboxylate radical of any one of Formula (V), Formula (V′), Formula C1, Formula 1C, Formula C2, Formula 2C, or Table 1 is attached to a hydroxyl radical of any one of Formula A, Formula A′, Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2 through a linker. In some embodiments, a hydroxyl radical of any one of Formula (V), Formula (V′), Formula C1, Formula 1C, Formula C2, Formula 2C, or Table 1 is attached to a carboxylate radical of any one of Formula A, Formula A′, Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2 through a linker. In some embodiments, a carboxylate radical of any one of Formula (V), Formula (V′), Formula C1, Formula 1C, Formula C2, Formula 2C, or Table 1 is attached to a carboxylate radical of any one of Formula A, Formula A′, Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2 through a linker.

In some embodiments, a carboxylate radical of any steroid described herein is attached to a carboxylate radical of any statin described herein through a linker.

In some embodiments, a hydroxyl radical of any steroid described herein is attached to a hydroxyl radical of any statin described herein through a linker.

In some embodiments, a carboxyl radical of any steroid described herein is attached to a hydroxyl radical of any statin described herein through a linker.

In some embodiments, a hydroxyl radical of any steroid described herein is attached to a carboxyl radical of any statin described herein through a linker.

In some embodiments, the linker is a bond. In some embodiments, the linker is not a bond. In some embodiments, the linker is —(O═C)CH₂—.

In some embodiments, a hydroxyl radical or a carboxylate radical of any one of Formula (V), Formula (V′), Formula C1, Formula 1C, Formula C2, Formula 2C, or a radical of a compound provided in Table 1 is attached to a hydroxyl radical or a carboxylate radical of another of any one of Formula A or Formula A′ through a linker. In some embodiments, a hydroxyl radical of any one of Formula (V′), Formula C1, Formula 1C, Formula C2, Formula 2C, or a radical of a compound provided in Table 1 is attached to a hydroxyl radical of any one of Formula B or Formula B′ through a linker. In some embodiments, a carboxylate radical of any one of Formula (V′), Formula C1, Formula 1C, Formula C2, Formula 2C, or a radical of a compound provided in Table 1 is attached to a hydroxyl radical of any one of Formula A or Formula A′ through a linker. In some embodiments, a hydroxyl radical of any one of Formula (V′), Formula C1, Formula 1C, Formula C2, Formula 2C, or a radical of a compound provided in Table 1 is attached to a carboxylate radical of any one of Formula A or Formula A′ through a linker. In some embodiments, a carboxylate radical of any one of Formula (V′), Formula C1, Formula 1C, Formula C2, Formula 2C, or a radical of a compound provided in Table 1 is attached to a carboxylate radical of any one of Formula B or Formula B′ through a linker.

In some embodiments, a carboxylate radical of any steroid described herein is attached to a carboxylate radical of rosuvastatin through a linker.

In some embodiments, a hydroxyl radical of any steroid described herein is attached to a hydroxyl radical of rosuvastatin through a linker.

In some embodiments, a carboxyl radical of any steroid described herein is attached to a hydroxyl radical of rosuvastatin through a linker.

In some embodiments, a hydroxyl radical of any steroid described herein is attached to a carboxyl radical of rosuvastatin through a linker.

In some embodiments, the linker is a bond. In some embodiments, the linker is not a bond.

In some embodiments, the linker is a bond, alkyl, heteroalkyl, or alkoxy, wherein the alkyl, heteroalkyl, or alkoxy is optionally substituted. In some embodiments, the alkyl, heteroalkyl, or alkoxy are each independently substituted with one or more substituent, each substituent being independently selected from the group consisting of oxo, —O— (e.g., hydroxyl or alkoxy), —S— (e.g., thiol or thioalkoxy), silicone, amino, alkyl, heteroalkyl, cycloalkyl, heterocycloalkyl, aryl or heteroaryl, wherein the alkyl, heteroalkyl, cycloalkyl, heterocycloalkyl, aryl or heteroaryl are optionally further substituted (e.g., with halogen or hydroxyl). In some embodiments, the linker is alkyl (alkylene) and the alkyl (alkylene) is substituted with one or more groups selected from —OH, halo, oxo, alkyl, heteroalkyl, cycloalkyl, and heterocycloalkyl.

In some embodiments, the linker comprises at least one oxo. In some embodiments, the linker comprises two oxo groups. In some embodiments, the linker comprises one or more ester, carbonate, anhydride, carbamate, ester, or any combination thereof. In some embodiments, the linker comprises at least one carbamate. In some embodiments, the linker comprises at least one carbonate. In some embodiments, the linker comprises at least one ester. In some embodiments, the linker comprises two or more esters.

In some embodiments, the linker comprises one or more linker groups, each linker group being independently selected from the group consisting of —O—, —S—, optionally substituted alkylene (e.g., alkenyl, alkynyl, branched (e.g., polypropylene), haloalkyl), optionally substituted heteroalkylene (e.g, polyTHF), and optionally substituted cycloalkylene. In some embodiments, the linker comprises one or more linker groups, each linker group being independently selected from the group consisting of alkyl, alkoxy, and cycloalkyl, wherein the alkyl, alkoxy, or cycloalkyl are optionally substituted.

In some embodiments, the linker comprises one or more linker groups selected from —O—S—, unsubstituted alkylene, (CH₂CH₂)n, (CHCH)n, O(CH₂CH₂O)n, (CH₂CH₂O)n, and (CH(CH₃)C(═O)O)ₙ, wherein n is 1-20. In some embodiments, the linker is unsubstituted alkylene, (CH₂CH₂)ₙ, (CHCH)ₙ, O(CH₂CH₂O)ₙ, (CH₂CH₂O)ₙ, (CH(CH₃)C(═O)O)ₙ, or (CH₂CH₂)ₙC═O(CH(CH₃)C(═O)O)ₙ, wherein n is 1-20.

In some embodiments, the linker is alkyl (alkylene) substituted with one or more groups selected from —OH, halo, oxo, alkyl, heteroalkyl, cycloalkyl, and heterocycloalkyl. In some embodiments, the linker is unsubstituted alkyl (alkylene). In some embodiments, the linker is heteroalkyl (heteroalkylene) substituted with one or more groups selected from halo or alkyl. In some embodiments, the linker is unsubstituted heteroalkyl (heteroalkylene). In some embodiments, the linker is selected from the group consisting of: —(CR₂)_{y}—, —O(CR₂)_{y}O—, —O(CR₂)_{y}—,—(CR₂)_{y}O—, and —O(CR₂CR₂O)_{y}—, wherein y is 1-10 and each R is independently selected from the group consisting of H, halogen, alkyl, or is taken together with another R to form an optionally substituted cycloalkyl. In some embodiments, each R is independently selected from H, alkyl, or is taken together with another R to form an optionally substituted cycloalkyl. In some embodiments, the one or more R is taken together with one or more other R to form a bridged cycloalkyl (e.g., a bridged cycloalkylene).

In some embodiments, a hydroxyl radical or a carboxylate radical of any one of Formula (V), Formula (V′), Formula C1, Formula 1C, Formula C2, Formula 2C, or Table 1 is attached to a hydroxyl radical or a carboxylate radical of another of any one of Formula A, Formula A, Formula B1, Formula 1B, Formula B1′, Formula 1B′, Formula B1′″, Formula 1B′″, Formula B2, Formula 2B, Formula B2′, Formula 2B′, Formula B2″, Formula 2B′″, or a radical of a compound provided in Table 2 through a linker. In some embodiments, a hydroxyl radical or a carboxylate radical of any one of Formula (V), Formula (V′), Formula C1, Formula 1C, Formula C2, Formula 2C, or Table 1 is attached to a hydroxyl radical or a carboxylate radical of any one of Formula A, Formula A, Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2 through a linker. In some embodiments, a hydroxyl radical of any one of Formula (V), Formula (V′), Formula C1, Formula 1C, Formula C2, Formula 2C, or Table 1 is attached to a hydroxyl radical of any one of Formula A, Formula A, Formula B1, Formula 1B, Formula B1′, Formula 1B′, Formula B1″, Formula 1B′″, Formula B2, Formula 2B, Formula B2′, Formula 2B′, Formula B2″, Formula 2B″, or a radical of a compound provided in Table 2 through a linker. In some embodiments, a hydroxyl radical of any one of Formula (V), Formula (V′), Formula C1, Formula 1C, Formula C2, Formula 2C, or Table 1 is attached to a carboxylate radical of any one of Formula A, Formula A, Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2 through a linker. In some embodiments, a hydroxyl radical of any one of Formula (V), Formula (V′), Formula C1, Formula 1C, Formula C2, Formula 2C, or Table 1 is attached to a carboxylate radical of any one of Formula A, Formula A, Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2 through a linker. In some embodiments, a carboxylate radical of any one of Formula (V), Formula (V′), Formula C1, Formula 1C, Formula C2, Formula 2C, or Table 1 is attached to a hydroxyl radical of any one of Formula A, Formula A, Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2 through a linker. In some embodiments, a carboxylate radical of any one of Formula (V), Formula (V′), Formula C1, Formula 1C, Formula C2, Formula 2C, or Table 1 is attached to a carboxylate radical of any one of Formula A, Formula A, Formula B1, Formula 1B, Formula B1′, Formula 1B′, Formula B1″, Formula 1B″, Formula B2, Formula 2B, Formula B2′, Formula 2B′, Formula B2″, Formula 2B″, or a radical of a compound provided in Table 2 through a linker. In some embodiments, the linker is a bond. In some embodiments, the linker is alkyl substituted with oxo.

In some embodiments, a hydroxyl radical or a carboxylate radical of any one of Formula (V), Formula (V′), Formula C1, Formula 1C, Formula C2, Formula 2C, or a radical of a compound provided in Table 1 is attached to a hydroxyl radical or a carboxylate radical of another of any one of Formula A or Formula A′ through a linker. In some embodiments, a hydroxyl radical or a carboxylate radical of any one of Formula (V), Formula (V′), Formula C1, Formula 1C, Formula C2, Formula 2C, or a radical of a compound provided in Table 1 is attached to a hydroxyl radical or a carboxylate radical of any one of Formula A or Formula A′ through a linker. In some embodiments, a hydroxyl radical of any one of Formula (V), Formula (V′), Formula C1, Formula 1C, Formula C2, Formula 2C, or a radical of a compound provided in Table 1 is attached to a hydroxyl radical of any one of Formula A or Formula A′ through a linker. In some embodiments, a hydroxyl radical of any one of Formula (V), Formula (V′), Formula C1, Formula 1C, Formula C2, Formula 2C, or a radical of a compound provided in Table 1 is attached to a carboxylate radical of any one of Formula A or Formula A′ through a linker. In some embodiments, a carboxylate radical of any one of Formula (V), Formula (V′), Formula C1, Formula 1C, Formula C2, Formula 2C, or a radical of a compound provided in Table 1 is attached to a hydroxyl radical of any one of Formula A or Formula A′ through a linker. In some embodiments, a carboxylate radical of any one of Formula (V), Formula (V′), Formula C1, Formula 1C, Formula C2, Formula 2C, or a radical of a compound provided in Table 1 is attached to a carboxylate radical of any one of Formula A or Formula A′ through a linker. In some embodiments, the linker is a bond. In some embodiments, the linker is alkyl substituted with oxo.

In some embodiments, the linker comprises at least one oxo. In some embodiments, the linker is oxo. In some embodiments, the linker comprises at least one carbamate. In some embodiments, the linker is a carbamate. In some embodiments, the linker comprises at least one ester. In some embodiments, the linker is an ester.

In some embodiments, the linker is hydrolyzed in a buffered solution. In some embodiments, the linker is hydrolytically labile. In some embodiments, the linker is hydrolyzed by water. In some embodiments, the linker is hydrolyzed by an enzyme. In some embodiments, the enzyme is a hydrolase (e.g., a protease or an esterase). In some embodiments, the enzyme is an esterase.

In some instances, longer linkers (e.g., at least 3-4 (e.g., carbon) atoms between an —O—) are preferred because, e.g., in some instances, shorter linkers result in increased melting point, increased T_{g}, increased crystallinity, decreased processability, or any combination thereof. In some instances, compounds comprising linkers having 7 or more carbon atoms (e.g., between an —O—) are not processable (e.g., because the compounds lack enough rigidity to form a sufficient crystal lattice).

In some embodiments, the linker is hydrolyzed in a buffered solution. In some embodiments, the linker is hydrolyzed by an enzyme. In some embodiments, the enzyme is a hydrolase (e.g., a protease or an esterase).

In some embodiments, a composition provided herein (e.g., an article or an implant) comprises a compound, or pharmaceutically acceptable salt thereof, having a structure provided herein, such as a structure provided in Table 4. In some embodiments, the compound comprises both a first radical and a second radical, which when combined are processable (e.g., wherein the radicals may or may not be processable themselves, when in their free form). In some embodiments, the compound, or pharmaceutically acceptable salt thereof, provided herein, such as a compound having a structure provided in Table 4, is a solid at a temperature of at least 20° C. (e.g., at least 30 C, at least 37 C, at least 40 C, at least 50 C, at least 70 C, at least 100 C, or the like). In some embodiments, the compound, or pharmaceutically acceptable salt thereof, provided herein, such as a compound having a structure provided in Table 4, is processable at a temperature of at least 20° C. (e.g., as described in the examples). In some embodiments, the compound, or pharmaceutically acceptable salt thereof, provided herein, such as a compound having a structure provided in Table 4, is processable into an article or implant (e.g., machined, molded, emulsion-processed, electrospun, electrosprayed, blow molded, or extruded to form a fiber, fiber mesh, woven fabric, non-woven fabric, film, surface coating, pellet, cylinder, rod, microparticle, nanoparticle, or another shaped article) at a temperature of at least 20° C.

In some embodiments, the compound, or pharmaceutically acceptable salt thereof, provided in Table 4 comprises a first radical and a second radical. In some embodiments, the first radical is a steroid radical and the second radical is a statin radical (or an active form thereof). In some embodiments, the compound in Table 4 is processable when the first radical (e.g., the steroid radical) and the second radical (e.g., the statin radical (or an active form thereof)) are joined by a linker. In some embodiments, the linker is a bond. In some embodiments, the linker is not a bond. In some embodiments, the linker is a linker described herein, such as a linker that is or comprises —(O═C)CH₂—.

TABLE 4

| Compound | | |
|---|---|---|
| Number | Structure | Name |
| 1 | 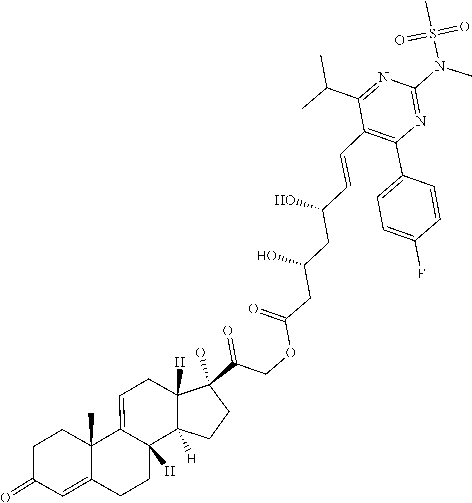 [SMILES: CC(C)c1nc(N(C)S(C)(=O)=O)nc(-c2ccc(F)cc2)c1/C=C/[C@@H](O)C[C@@H](O)CC(=O)OCC(=O)[C@@]1(O)CC[C@H]2[C@@H]3CCC4=CC(=O)CC[C@]4(C)C3=CC[C@@H]21] | 2-((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethyl (3R,5S,E)-7-(4-(4- fluorophenyl)-6- isopropyl-2-(N- methylmethylsulfonamido) pyrimidin-5-yl)-3,5- dihydroxyhept-6-enoate |
| 2 | 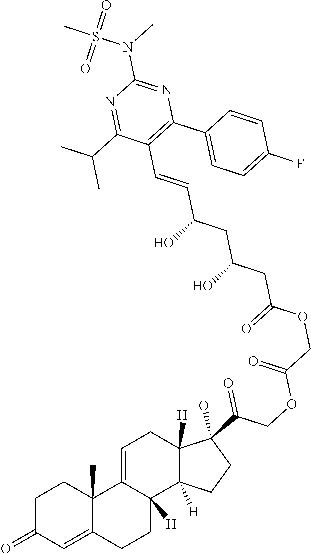 | 2-(2- ((8S,10S,13S,14S,17R)- 17-hydroxy-10,13- dimethyl-3-oxo- 2,3,6,7,8,10,12,13,14,15, 16,17-dodecahydro-1H- cyclopenta[a]phenanthren- 17-yl)-2-oxoethoxy)-2- oxoethyl (3R,5S,E)-7-(4- (4-fluorophenyl)-6- isopropyl-2-(N- methylmethylsulfonamido) pyrimidin-5-yl)-3,5- dihydroxyhept-6-enoate |

Provided in some embodiments herein are articles or implants that demonstrate unique properties for drug release applications. In some embodiments, the release of a (e.g., steroid or statin, or an active form thereof) radical in its free form or a compound provided herein from an article or implant provided herein is beneficial (e.g., for drug release applications). In some embodiments, a composition provided herein has near zero-order release kinetics for an extended period of time (e.g., 1 day or more, 2 days or more, 3 days or more, 4 days or more, 5 days or more, 6 days or more, 7 days or more, 14 days or more, or 30 days or more) (e.g., in an individual in need thereof). In some embodiments, a composition provided herein releases at least one radical in its free form or a compound provided herein by surface erosion from the article (e.g., pellet) or implant provided herein.

In certain instances, provided herein is an article or implant comprising any compound described herein, or pharmaceutically acceptable salt thereof, such as a compound having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, or a compound provided in Table 1 or Table 2.

In certain instances, provided herein is an article or implant comprising a compound, or pharmaceutically acceptable salt thereof, having a structure represented by any one of Formula II, Formula ID, Formula IIC, Formula IIC′, Formula IIA, Formula IIA′, Formula IIB, Formula IIB′, or a compound provided in Table 4.

In some embodiments, a compound, or pharmaceutically acceptable salt thereof, provided herein, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, or a compound provided in Table 4 is a symmetrical compound (e.g., the first radical and the second radical have the same biological function (e.g., alone or in combination treat the same indication (e.g., described herein)) in their free form). In some embodiments, the symmetrical compound comprises a first radical and a second radical that each have the same biological function (e.g., alone or in combination treat the same indication (e.g., described herein (e.g., AMD))) in their free form. In some embodiments, the symmetrical compound comprises a first radical and a second radical that are each a cholesterol-lowering agent in their free form. In some embodiments, the symmetrical compound comprises a first radical and a second radical that (in combination and in their free from) treat an indication described herein (e.g., AMD).

In some embodiments, a compound, or pharmaceutically acceptable salt thereof, provided herein, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, or a compound provided in Table 4 is an asymmetrical compound (e.g., the first radical and the second radical have a different structure, a different biological function, or a combination thereof). In some embodiments, the asymmetrical compound comprises a first radical and a second radical that each have the same biological function (e.g., alone or in combination treat the same indication (e.g., described herein (e.g., AMD))))) in their free form. In some embodiments, the asymmetrical compound comprises a first radical and a second radical that each have a different biological function in their free form. In some embodiments, the asymmetrical compound comprises a first radical or a second radical that is an anti-inflammatory agent in their free form. In some embodiments, the asymmetrical compound comprises a first radical and/or a second radical that are each independently a cholesterol-lowering agent in their free form. In some embodiments, the asymmetrical compound comprises a first radical and a second radical that (in combination and in their free from) treat an indication described herein. In some embodiments, the asymmetrical compound comprises a first radical that is a steroid in its free form and a second radical that is a cholesterol lowering agent in its free form. In some embodiments, the asymmetrical compound comprises a first radical and a second radical that are each a cholesterol lowering agent in their free form. In some embodiments, the first radical and the second radical have a different structure (e.g., as a radical or in their free form). In some embodiments, the asymmetrical compound comprises a first radical and a second radical that (in combination and in their free from) treat an indication described herein (e.g., AMD).

In some embodiments, a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, or a compound provided in Table 4 provided herein is a solid, such as at a physiological temperature (e.g., having a melting point (Tₘ) and/or glass transition temperature (T_{g}) of at least 37° C.). In some embodiments, the solid is a crystalline solid, a film, a glass, or an amorphous solid (e.g., at a temperature of at least 37° C.). In some embodiments, the compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, or Formula IB′, provided herein is not an oil.

In some embodiments, an article or implant provided herein, comprising a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, has a crystallinity of at most 15% (e.g., determined by PXRD, DSC, or polarized light microscopy). In some embodiments, an article or implant provided herein, comprising a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, is substantially crystalline (e.g., determined by PXRD, DSC, or polarized light microscopy). In some embodiments, an article or implant provided herein, comprising a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, is crystalline (e.g., determined by PXRD, DSC, or polarized light microscopy). In some embodiments, an article or implant provided herein, comprising a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, is amorphous (e.g., determined by PXRD, DSC, or polarized light microscopy).

In some embodiments, an article or implant provided herein, comprising a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, has a thermal melting point (Tₘ) that is greater than or equal to the glass transition temperature (T_{g}). In some embodiments, an article or implant provided herein, comprising a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, has a melting point of less than or equal to either one or both of the first and/or second radicals (e.g., in their free form) of the compound.

In some embodiments, an article or implant provided herein, comprising a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, has a melting point of at least 37° C. In some embodiments, an article or implant provided herein, comprising a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, has a melting point of at least 100° C. In some embodiments, an article or implant provided herein, comprising a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, has a melting point of at least 160° C. In some embodiments, an article or implant provided herein, comprising a compound, or pharmaceutically acceptable salt thereof, having a structure represented by any one of Formula 1, Formula I, Formula (ID), Formula IC, Formula IC′, Formula IC1, Formula IC1′, Formula D, Formula D′, Formula IA, Formula IA′, Formula IB, Formula IB′, Formula II, Formula ID, Formula IIC, Formula IIC′, Formula IIA, Formula IIA′, Formula IIB, Formula IIB′, has a melting point of at most 220° C.

In some embodiments, the composition (e.g., article) provided herein comprises less than 5 wt. %, less than 2 wt. %, or less than 1 wt. % of a controlled release excipient. In some embodiments, the composition (e.g., article) provided herein is free of a controlled release excipient.

In some embodiments, the implant or article comprises at least 50 wt. % (at least 60 wt. %, at least 70 wt. %, at least 80 wt. %, at least 90 wt. %, at least 95 wt. %, at least 98 wt. %, or the like) of a compound provided herein. In certain instances, the implant or article releases a (e.g., active) group therefrom, such as when implanted into or otherwise administered to an individual (or when placed into an aqueous medium (e.g., aqueous buffer solution), serum, or other physiological medium, such as at a physiological temperature, such as 37° C.). In some instances, a (e.g., active) group released is the free form of the first radical, the second radical, and/or the therapeutic agent. In certain instances, a (e.g., active) group released from article or implant is an active fragment or metabolite of the first and/or second radical. In some embodiments, the implant or article undergoes surface erosion to release a compound, the first radical, and/or the second radical (or an (e.g., active) fragment or radical thereof). In some embodiments, the first radical and the second radical are released from the pharmaceutical implant or article at near zero-order in solution (e.g., buffer solution, serum, biological environment, in vivo, or the like). In some embodiments, the first radical and the second radical (or an (e.g., active) fragment or metabolite thereof) are released from the pharmaceutical implant or article at 37° C. in 100% bovine serum or at 37° C. in phosphate buffered saline (PBS) at a rate such that t₁₀ is greater than or equal to 1/10 of t₅₀.

In some instances, t₅₀ is the time at which 50% of the releasable drug has been released from a composition (e.g., an article or an implant) provided herein. In some instances, time t₁₀ is, correspondingly, the time at which 10% of the releasable drug has been released from a composition (e.g., an article or an implant) provided herein. In some instances, such as when the release curve is perfectly linear, t₁₀=1/5 of t₅₀. In some instances, such as when there is an initial burst of released drug, t₁₀ is much less than 1/5 of t₅₀. In some instances, t₁₀ can be equal to or greater than 1/10 of t₅₀. Drug release from a composition (e.g., an article or an implant) provided herein can be measured at 37° C. in 100% bovine serum, or at 37° C. in PBS (phosphate buffered saline), as described in the Examples.

In some embodiments, the pharmaceutical implant, article, or composition is biodegradable.

In some embodiments, the pharmaceutical implant, article, or composition is at least partially biodegradable.

In some embodiments, the pharmaceutical implant, article, or composition is non-biodegradable.

In some embodiments, a compound (a compound having the structure of any one of Formula 1, Formula I, Formula (ID), Formula IC, Formula IC′, Formula IC1, Formula IC1′, Formula D, Formula D′, Formula IA, Formula IA′, Formula IB, or Formula IB′, or a pharmaceutically acceptable salt or solvate thereof) further comprises an amount of a free form of any radical provided herein, or a combination thereof, such as a free form of a radical having the (e.g., steroid and/or statins) structure of any one of Formula 1, Formula I, Formula (ID), Formula IC, Formula IC′, Formula IC1, Formula IC1′, Formula D, Formula D′, Formula IA, Formula IA′, Formula IB, or Formula IB′, wherein the free form (e.g., —COO— or —O— thereof) has a negative charge (e.g., as —O—) or an H (e.g., as —OH), rather than being connected to a linker and/or other (first or second) radical). In some embodiments, a compound provided herein comprises a (e.g., weight or molar) ratio of a compound provided herein to a free form of any radical provided herein, or a combination thereof, such as a free form of a radical having the structure of any one of Formula 1, Formula I, Formula (ID), Formula IC, Formula IC′, Formula IC1, Formula IC1′, Formula D, Formula D′, Formula IA, Formula IA′, Formula IB, or Formula IB′, or a pharmaceutically acceptable salt thereof (e.g., wherein the free form (e.g., —COO— or —O— thereof) has a negative charge (e.g., as —O—) or an H (e.g., as —OH)), rather than being connected to a linker and/or other (first or second) radical, of about 1:99 to about 100:0 (e.g., the amount of the free form of the radical relative to the overall amount of free form of the radical plus the conjugate is between 0% (weight or molar) and 99%). In some embodiments, the relative amount of the free form of the radical is 0% to about 50%, such 0% to about 20%, 0% to about 10%, about 0.1% to about 10%, about 0.1% to about 5%, less than 5%, less than 2.5%, less than 2%, or the like (percentages being weight/weight or mole/mole percentages). Further, in some instances, compounds provided herein release free form of any radical provided herein, or a combination thereof, such as a free form of a structure of a compound having the structure of any one of Formula 1, Formula I, Formula (ID), Formula IC, Formula IC′, Formula IC1, Formula IC1′, Formula D, Formula D′, Formula IA, Formula IA′, Formula IB, or Formula IB′ (e.g., wherein the free form (e.g., —COO— or —O— thereof) has a negative charge (e.g., as —O—) or H (e.g., as —OH), rather than being connected to a linker and/or other (first or second) radical)), such as when administered to an individual (e.g., intraocular administration).

In certain instances, a composition provided herein further comprises an amount of a free form of a radical, such as having the structure represented by any one of Formula 1, Formula I, Formula (ID), Formula IC, Formula IC′, Formula IC1, Formula IC1′, Formula D, Formula D′, Formula IA, Formula IA′, Formula IB, or Formula IB′, or the like (such as wherein the free form is the radical, wherein R is a negative charge or an H). In some embodiments, a composition provided herein comprises a (e.g., weight or molar) ratio of a compound provided herein to a free form of a radical having the structure represented by any one of Formula 1, Formula I, Formula (ID), Formula IC, Formula IC′, Formula IC1, Formula IC1′, Formula D, Formula D′, Formula IA, Formula IA′, Formula IB, or Formula IB′, or a pharmaceutically acceptable salt thereof (e.g., wherein R is a negative charge or an H) is about 1:99 to about 100:0 (e.g., the amount of the free form of the radical relative to the overall amount of free form of the radical plus the conjugate is between 0% (weight or molar) and 99%). In some embodiments, the relative amount of the free form of the radical is 0% to about 50%, such as about 0% to about 20%, 0% to about 10%, about 0.1% to about 10%, about 0.1% to about 5%, less than 5%, less than 2.5%, less than 2%, or the like (percentages being weight/weight or mole/mole percentages). Further, in some instances, compositions provided herein release free form of a radical of a compound having the structure represented by any one of Formula 1, Formula I, Formula (ID), Formula IC, Formula IC′, Formula IC1, Formula IC1′, Formula D, Formula D′, Formula IA, Formula IA′, Formula IB, or Formula IB′, (e.g., wherein R is a negative charge or H), such as when administered to an individual (e.g., ocular (e.g., intraocular) administration).

In some embodiments, a compound (a compound having the structure of any one of Formula II, Formula ID, Formula IIC, Formula IIC′, Formula IIA, Formula IIA′, Formula IIB, Formula IIB′, or a compound provided in Table 4, or a pharmaceutically acceptable salt or solvate thereof) further comprises an amount of a free form of any radical provided herein, or a combination thereof, such as a free form of a radical having the (e.g., steroid and/or statins) structure of any one of Formula II, Formula ID, Formula IIC, Formula IIC′, Formula IIA, Formula IIA′, Formula IIB, Formula IIB′, or a compound provided in Table 4, wherein the free form (e.g., —COO— or —O-thereof) has a negative charge (e.g., as —O—) or an H (e.g., as —OH), rather than being connected to a linker and/or other (first or second) radical). In some embodiments, a compound provided herein comprises a (e.g., weight or molar) ratio of a compound provided herein to a free form of any radical provided herein, or a combination thereof, such as a free form of a radical having the structure of any one of Formula II, Formula ID, Formula IIC, Formula IIC′, Formula IIA, Formula IIA′, Formula IIB, Formula IIB′, or a compound provided in Table 4, or a pharmaceutically acceptable salt thereof (e.g., wherein the free form (e.g., —COO— or —O— thereof) has a negative charge (e.g., as —O—) or an H (e.g., as —OH)), rather than being connected to a linker and/or other (first or second) radical, of about 1:99 to about 100:0 (e.g., the amount of the free form of the radical relative to the overall amount of free form of the radical plus the conjugate is between 0% (weight or molar) and 99%). In some embodiments, the relative amount of the free form of the radical is 0% to about 50%, such 0% to about 20%, 0% to about 10%, about 0.1% to about 10%, about 0.1% to about 5%, less than 5%, less than 2.5%, less than 2%, or the like (percentages being weight/weight or mole/mole percentages). Further, in some instances, compounds provided herein release free form of any radical provided herein, or a combination thereof, such as a free form of a structure of a compound having the structure of any one of Formula II, Formula ID, Formula IIC, Formula IIC′, Formula IIA, Formula IIA′, Formula IIB, Formula IIB′, or a compound provided in Table 4 (e.g., wherein the free form (e.g., —COO— or —O— thereof) has a negative charge (e.g., as —O—) or H (e.g., as —OH), rather than being connected to a linker and/or other (first or second) radical)), such as when administered to an individual (e.g., intraocular administration).

In certain instances, a composition provided herein further comprises an amount of a free form of a radical, such as having the structure represented by any one of Formula II, Formula ID, Formula IIC, Formula IIC′, Formula IIA, Formula IIA′, Formula IIB, Formula IIB′, or a compound provided in Table 4, or the like (such as wherein the free form is the radical, wherein R is a negative charge or an H). In some embodiments, a composition provided herein comprises a (e.g., weight or molar) ratio of a compound provided herein to a free form of a radical having the structure represented by any one of Formula II, Formula ID, Formula IIC, Formula IIC′, Formula IIA, Formula IIA′, Formula IIB, Formula IIB′, or a compound provided in Table 4, or a pharmaceutically acceptable salt thereof (e.g., wherein R is a negative charge or an H) is about 1:99 to about 100:0 (e.g., the amount of the free form of the radical relative to the overall amount of free form of the radical plus the conjugate is between 0% (weight or molar) and 99%).

In some embodiments, the relative amount of the free form of the radical is 0% to about 50%, such as about 0% to about 20%, 0% to about 10%, about 0.1% to about 10%, about 0.1% to about 5%, less than 5%, less than 2.5%, less than 2%, or the like (percentages being weight/weight or mole/mole percentages). Further, in some instances, compositions provided herein release free form of a radical of a compound having the structure represented by any one of Formula II, Formula ID, Formula IIC, Formula IIC′, Formula IIA, Formula IIA′, Formula IIB, Formula IIB′, or a compound provided in Table 4, (e.g., wherein R is a negative charge or H), such as when administered to an individual (e.g., ocular (e.g., intraocular) administration).

In some embodiments, less than or equal to 20% (w/w) (e.g., less than 20% w/w, less than 15% w/w, less than 10% w/w, or less than 5% w/w) of D1 and/or D2 (e.g., as a percentage of the total article or implant) are released from the article or implant in their free form at 37° C. in PBS over a period 5 days or more (e.g., more than 5 days, more than 7 days, or more than 10 days). In some embodiments, less than or equal to 20% (w/w) (e.g., less than 20% w/w, less than 15% w/w, less than 10% w/w, or less than 5% w/w) of the at least one therapeutic agent, D1, and/or D2 (e.g., as a percentage of the total article or implant) are released from the article or implant in their free form at 37° C. in PBS over a period 5 days or more (e.g., more than 5 days, more than 7 days, or more than 10 days).

In some embodiments, the article or implant has a melting point of at least 37° C. (e.g., at least 100° C., at least 160° C., or at least 200° C.). In some instances, the article or implant has a melting point of at most 220° C. In some instances, an article or implant with a melting point greater than 220° C. decomposes (e.g., and is not processable into an article (e.g., a pellet) or implant provided herein) subsequent to heat processing or solvent processing methods provided herein. In some instances, the article or implant provided herein has a melting point that is less than or equal to either one or both of the first and/or second radicals (e.g., in their free form) of the article or implant.

In some embodiments, the release profile of the article or implant provided herein is modified by the linker (e.g., L). For example, in certain instances, a linker that is less susceptible to hydrolysis (e.g., steric effects, electronic effects, etc.) provides an article or implant that releases (e.g., from the article or implant) a compound provide herein (e.g., a radical thereof in its free form) at a slower (e.g., extended-release) rate compared to an article or implant that is more susceptible to hydrolysis (e.g., providing an article or implant that releases (e.g., from the article or implant) compound provided herein (e.g., a radical thereof in its free form) at a faster (e.g., immediate-release) rate).

In some embodiments, either one or both of the first and/or second radicals of a composition provided herein (e.g., an implant or article), having a structure represented by any one of Formula 1, Formula I, Formula (ID), Formula IC, Formula IC′, Formula IC1, Formula IC1′, Formula D, Formula D′, Formula IA, Formula IA′, Formula IB, or Formula IB′, is released (e.g., in their free form) from the composition, the release being controlled release (e.g., near zero-order) and/or extended release. In some embodiments, either one or both of the first and/or second radicals of a composition provided herein (e.g., an implant or article), having a structure represented by any one of Formula 1, Formula I, Formula (ID), Formula IC, Formula IC′, Formula IC1, Formula IC1′, Formula D, Formula D′, Formula IA, Formula IA′, Formula IB, or Formula IB′, is released (e.g., in their free form) from the composition for at least 15 days (e.g., a buffer solution, serum, biological environment (e.g., in the eye), in vivo, or the like).

In some embodiments, either one or both of the first and/or second radicals of a composition provided herein (e.g., an implant or article), having a structure represented by any one of Formula II, Formula ID, Formula IIC, Formula IIC′, Formula IIA, Formula IIA′, Formula IIB, Formula IIB′, or a compound provided in Table 4, is released (e.g., in their free form) from the composition, the release being controlled release (e.g., near zero-order) and/or extended release. In some embodiments, either one or both of the first and/or second radicals of a composition provided herein (e.g., an implant or article), having a structure represented by any one of Formula II, Formula ID, Formula IIC, Formula IIC′, Formula IIA, Formula IIA′, Formula IIB, Formula IIB′, or a compound provided in Table 4, is released (e.g., in their free form) from the composition for at least 15 days (e.g., a buffer solution, serum, biological environment (e.g., in the eye), in vivo, or the like).

In some instances, a compound provided herein is administered as a pure (e.g., greater than 98 wt. %, greater than 99 wt. %, about 100 wt. %) chemical. In other embodiments, a composition described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

In some embodiments, the compound is formed into a surface coating, a drug depot, an article, an implant, or other material or form described herein.

In some embodiments, the compound is formed into an implantable article or an implant. In some instances, the implantable article or the implant consists essentially of the compound.

In some embodiments, the pharmaceutical composition is suitable for ophthalmic administration. In some embodiments, the pharmaceutical composition is suitable for intraocular administration. In some embodiments, intraocular administration is administration in the eye, such as intracameral, intravitreal, suprachoroidal, punctal, retrobulbar, or subconjunctival administration.

In some embodiments, the pharmaceutical composition is suitable for ocular administration. In some embodiments, the pharmaceutical composition is suitable for intraocular administration.

In certain instances, any compound and/or composition (e.g., article, implant) provided herein is substantially pure, in that it contains less than about 5%, or less than about 1%, or less than about 0.1%, of other organic small molecules, such as impurities, unreacted intermediates or (e.g., processing and/or synthesis) by-products that are created, for example, in one or more of the steps of a synthesis method and/or processing step (such as heat processing, solvent processing, and/or sterilization).

In some embodiments, an implant or article provided herein comprises more than or equal to 25 wt. % (50 wt. % or more, 75 wt. % or more, 90 wt. % or more, 95 wt. % or more, or 99 wt. % or more) of a compound, and/or pharmaceutically acceptable salt thereof, provided herein. In some embodiments, an implant or article provided herein comprises less than or equal to 25 wt. % of a compound, and/or pharmaceutically acceptable salt thereof, provided herein.

In some embodiments, an implant or article provided herein comprises more than or equal to 50 wt. % (at least 60 wt. %, at least 70 wt. %, at least 80 wt. %, at least 90 wt. %, at least 95 wt. %, at least 98 wt. %, or more) of a compound, and/or pharmaceutically acceptable salt thereof, provided herein. In some embodiments, an implant or article provided herein comprises less than or equal to 50 wt. % (at most 40 wt. %, at most 30 wt. %, at most 20 wt. %, at most 10 wt. %, at most 5 wt. %, at most 1 wt. %, or less) of a compound, and/or pharmaceutically acceptable salt thereof, provided herein.

In some embodiments, the implant or article comprises at least 50 wt. % (at least 60 wt. %, at least 70 wt. %, at least 80 wt. %, at least 90 wt. %, at least 95 wt. %, at least 98 wt. %, or the like) of the compound and/or pharmaceutically acceptable salt thereof. In some instances, an article or implant provided herein comprises at least 50 wt. % of a compound as described herein. In some instances, an article or implant provided herein comprises at least 70 wt. % of a compound as described herein. In some instances, an article or implant provided herein comprises at least 90 wt. % of a compound as described herein. In some instances, an article or implant provided herein comprises at least 95 wt. % of a compound as described herein. In some instances, an article or implant provided herein comprises at least 99 wt. % of a compound as described herein. In some instances, an article or implant provided herein comprises an additional component, such as up to 20 wt. %, 15 wt. %, 10 wt. %, 5 wt. %, 1 wt. %, 0.1 wt. %, 0.01 wt. %, or less of the additional component. In some embodiments, an article or implant provided herein comprises up to 5 wt. % (e.g., up to 1 wt. %, up to 0.1 wt. %, or less) a first radical in its free form (e.g., a steroid (such as described herein)), a second radical in its free form (e.g., a statin (such as described herein), or a combination thereof (e.g., as an impurity, such as residual from a manufacturing process, such as provided herein). In some embodiments, an article or implant provided herein comprises up to 5 wt. % (e.g., up to 1 wt. %, up to 0.1 wt. %, or less) of an impurity, such as residual from a manufacturing process, such as provided herein. In some embodiments, an article or implant provided herein comprises up to 5 wt. % (e.g., up to 1 wt. %, up to 0.1 wt. %, or less) of a steroid (as described herein).

In some embodiments, an implant or article provided herein comprises about 70 wt. % or more of a compound provided herein.

In some embodiments, an implant or article provided herein comprises about 80 wt. % or more of a compound provided herein.

In some embodiments, an implant or article provided herein comprises about 90 wt. % or more of a compound provided herein.

In some embodiments, an implant or article provided herein undergoes surface erosion to release a compound described herein. In some embodiments, a compound provided herein is released from the pharmaceutical implant or article at near zero-order in solution (e.g., buffer solution, serum, biological environment, in vivo, or the like). In some embodiments, a compound provided herein is released from the pharmaceutical implant or article at 37° C. in 100% bovine serum or at 37° C. in phosphate buffered saline (PBS) at a rate such that t₁₀ is greater than or equal to 1/10 of t₅₀.

In certain embodiments, the implant or article releases a (e.g., active) group therefrom, such as when implanted into or otherwise administered to an individual (or when placed into an aqueous medium (e.g., aqueous buffer solution), serum, or other physiological medium, such as at a physiological temperature, such as 37° C.). In some instances, a (e.g., active) group released is the free form of the first radical and/or the second radical. In certain instances, a (e.g., active) group released from the compound is an active fragment or metabolite of the first and/or second radical. In some embodiments, the implant or article undergoes surface erosion to release the compound, the first radical, and/or the second radical (or an (e.g., active) fragment or radical thereof). In some embodiments, first radical and the second radical are released from the pharmaceutical implant or article at near zero-order in solution (e.g., buffer solution, serum, biological environment, in vivo, or the like). In some embodiments, the first radical and the second radical (or an (e.g., active) fragment or metabolite thereof) are released from the pharmaceutical implant or article at 37° C. in 100% bovine serum or at 37° C. in phosphate buffered saline (PBS) at a rate such that t₁₀ is greater than or equal to 1/10 of t₅₀.

In certain aspects, a pharmaceutical composition provided herein includes an article or implant in the form of fibers, fiber meshes, woven fabrics, non-woven fabrics, pellets, cylinders, rods, hollow tubes, microparticles, nanoparticles, or other shaped articles. In some embodiments, a pellet is rounded, spherical, cylindrical, or a combination thereof. In some embodiments, the pellet has a mean diameter of 0.01 mm or more (e.g., 0.1 mm or more or 1 mm or more). In some embodiments, the pellet has a mean diameter of 5 mm or less (e.g., 5 mm or less, 0.5 mm or less, or 0.05 or less). In some embodiments, the pellet has a mean diameter from about 0.2 to 5 mm, e.g., from about 0.2 to 1 mm, from about 0.2 to 2 mm, from about 0.3 to 3 mm, from about 1.5 to 5 mm, from about 2 to 5 mm, from about 2.5 to 5 mm, from about 3 to 5 mm, from about 3.5 to 5 mm, from about 4 to 5 mm, or from about 4.5 to 5 mm. In some embodiments, a pharmaceutical composition provided herein has a non-circular shape that affects, e.g., increases, the surface area (e.g., extruded through star-shaped dye or any other form shaping process with or without a dye mold). In some embodiments, suitable pharmaceutical compositions for use with this disclosure are small regularly or irregularly shaped particles, which can be solid, porous, or hollow.

In some embodiments, certain forms of pharmaceutical compositions described herein (e.g., fibers, fiber meshes, woven fabrics, non-woven fabrics, pellets, cylinders, rods, hollow tubes, microparticles (e.g., microbeads), nanoparticles (e.g., nanobeads), or other shaped articles) provide a controllable surface area. In some embodiments, a pharmaceutical composition provided herein is injected into an individual (e.g., in need thereof) and does not require removal after completion of drug release. In certain instances, methods provided herein do not require (or comprise) removal of an article or implant, or residual materials or components thereof (e.g., because the implant is completely or almost completely (e.g., bio- or physiologically) degraded or degradable (e.g., at least 80 wt. %, at least 85 wt. %, at least 90 wt. %, at least 95 wt. %, at least 98 wt. %, at least 99 wt. %, or the like)).

In some embodiments, the implants, articles, or compositions described herein are amorphous. In some embodiments, the implants, articles, or compositions described herein are formed by heat-based and solvent based processing methods. Non-limiting examples of heat processing methods include heat molding, injection molding, extrusion, 3D printing, melt electrospinning, fiber spinning, fiber extrusion, and/or blow molding. Non-limiting examples of solvent processing include coating, micro printing, dot printing, micropatterning, fiber spinning, solvent blow molding, emulsion-based, electrospraying, and electrospinning. In some embodiments, processing methods to form an intermediate glassy state of any of the above heat and solvent based methods as well as heat and solvent based methods that lead to glassy state material with no defined shape (e.g. spray drying, lyophilization, powder melting, etc.).

In some embodiments, a glassy state is an amorphous solid including greater than 70%, 80%, 90%, 95%, 98%, or 99% (w/w) of compositions, articles, or implants described herein. In some embodiments, the compositions, articles, or implants described herein exhibit a glass transition temperature above 38° C. In the glassy state, as measured by differential scanning calorimetry (DSC), X-ray powder diffraction (XRD), or polarized light microscopy (PLM), the level of crystallinity is, for example, from 0-15% (e.g., less than 1%, 0-1%, 0-3%, 0-5%, 0-7%, 0-9%, 0-10%, or 0-13%). In some embodiments, glass formulations are formed using heat processing or solvent processing methods described herein (e.g., in the examples).

In some embodiments, the pharmaceutical compositions described herein are prepared by electrospinning. In some embodiments, the pharmaceutical compositions of the disclosure are dissolved in a solvent (e.g., acetone) at concentrations ranging from, e.g., 10-30% w/v, and are electrosprayed to form micro- and nanobeads. In some embodiments, the solution is loaded into a syringe and injected at a rate (e.g., 0.5 mL/h) onto a stationary collection plate. In some embodiments, a potential difference (e.g., 18 kV) is maintained between the needle and collecting surface. For example, in certain instances, a concentration of 10% w/v is used to obtain nanoparticles. In other embodiments, a concentration of 30% w/v is used to obtain microbeads.

The pharmaceutical compositions of the disclosure are dissolved in a solvent (e.g., THF, or 1:1 ratio of DCM/THF). In some embodiments, the solution is loaded into a syringe and injected at a rate (e.g., 0.5 mL/h) onto a cylindrical mandrel rotating at a particular rotational speed, e.g., 1150 rpm, to obtain aligned fibers, or onto a stationary collector surface to obtain unaligned fibers. In some embodiments, a potential difference (e.g., 18 kV or 17 kV) is maintained between the needle and collecting surface for aligned and random fibers.

In some embodiments, provided herein is a method of forming an article or implant provided herein comprising producing a glassy state (e.g., an intermediate glassy state or a melt) of a compound (e.g., a crystalline form (e.g., a solid or a powder)) provided herein using heat or solvent. In other embodiments, fibers are prepared from the melt or the glass at elevated temperatures, the glassy state intermediate, or from the solution by dissolving the pharmaceutical compositions described herein in a solvent (e.g., DCM, THF, or chloroform). In some instances, compositions provided herein are heat processed from the melt state. In some instances, compositions provided herein are heat processed by heat spinning from the glassy state.

In some embodiments, the viscous melt, intermediate, or solution is fed through a spinneret and fibers are formed upon cooling (melt or heat spinning) or following solvent evaporation with warm air as the compound exits the spinneret (dry spinning). In some embodiments, wet spinning and gel spinning are used to produce the fibers disclosed herein. In some embodiments, heat spinning is performed with a glassy state intermediate and heated above the glass transition temperature (T_{g}), obtaining the viscous fluid to extrude/spin instead of the melt. Alternatively, tweezers may be dipped into melted material or concentrated solutions and retracted slowly in order to pull fibers. The rate of pulling and distance pulled may be varied to yield fibers and columnar structures of different thickness.

In some embodiments, micro-particles or nano-particles made from the pharmaceutical composition are formed using an emulsion process. In some embodiments, the micro or nano-particles are made by recrystallization. In some embodiments, the pharmaceutical composition is dissolved in an organic solvent (e.g. DCM, THF, etc.). In some embodiments, a surfactant (e.g. SDS, PVA, etc.) is added (e.g. 1%) to the solution/mixture. In some embodiments, the resulting mixture is stirred for the appropriate time at room temperature to form an emulsion. In some embodiments, the emulsion is subsequently added to Milli-Q water under stirring for an appropriate time (e.g., 1 h) to remove residual solvent. The resulting micro- or nano-particles may be collected by centrifugation and dried.

In some embodiments, injectable cylinders made from a pharmaceutical composition described herein is formed by heat extrusion. In some embodiments, the pharmaceutical composition is loaded into a hot melt extruder, heated to a temperature above the melting point (e.g., for crystalline compositions) or glass transition temperature (e.g., for pre-melted or amorphous compositions), and extruded using (i) a compressive force to push the material through the nozzle and (ii) a tensile force (or gravity) to pull the material out of the extruder. The extrudate may be cut to the desired length for suitable drug dosing for a medical indication.

In some embodiments, a milling process is used to reduce the size of an article described to form sized particles, e.g., beads, in the micrometer (microbeads) to nanometer size range (nanobeads). The milling process may be performed using a mill or other suitable apparatus. In some embodiments, dry and wet milling processes, such as, for example, jet milling, cryo-milling, ball milling, media milling, sonication, and homogenization are used in methods described herein. In some embodiments, heating of the milled microparticle above the T_{g} is performed to achieve a spherical shape. In some embodiments, particles with non-spherical shapes are used as milled.

In certain instances, a composition described herein has a limited window (e.g., short timeframe of seconds to minutes) of thermal stability, whereby the purity of the dimer is affected (e.g., minimally) at elevated temperatures. In some embodiments, an intermediate glassy state form (e.g., film, surface coating, pellet, micro-particles, or other shaped article) is made to avoid decomposition. In some embodiments, heat or solvent processing is used to remove or reduce the crystallinity of the material to form a glassy state composition. In some embodiments, the glassy state composition is heat processed at a lower temperature (e.g., processing just above the glass transition temperature (T_{g}), and below the melt temperature (Tₘ)). In some embodiments, the lower temperature allows for a longer timeframe for heat processing the glassy state material into the final shaped article, while reducing the impact of processing conditions on the purity of the compound in the article.

In some embodiments, an article or implant provided herein is formulated for administration by injection. In some instances, the injection formulation is a solid formulation. In some instances, the injection formulation is a non-aqueous formulation.

In some embodiments, an article or implant provided herein is formulated for administration by instillation. In some instances, the instillation formulation is a solid formulation. In some instances, the instillation formulation is a non-aqueous formulation.

In certain embodiments, a pharmaceutical compositions described herein has a controlled release profile (e.g., by surface erosion). In some embodiments, the surface erosion allows the article or implant to maintain its physical form, while gradually decreasing in size as the surface erodes (e.g., at a constant rate), rather than by, for example, bulk erosion that is characteristic of some polymer-based drug release vehicles (e.g., polylactic/glycolic acid). In some embodiments, the surface erosion inhibits burst release and/or reduces the formation of inflammatory particulates (e.g., no or minimal crystalline particulates are formed or released from the articles or implants when drug is released as described herein). In some embodiments, compositions described herein are delivered over a period of time. For example, a slower and steadier rate of delivery (e.g., release of less than 10% of the first radical or the second radical in their free form (as a percentage of the total drug, the first radical or the second radical in their free form, present in the article or implant) at 37° C. in 100% bovine serum over 5 days) results in a reduction in the frequency with which the pharmaceutical composition is administered to a subject and/or improve the safety profile of the drug. In some embodiments, the drug release is tailored to avoid side effects of slower and longer release of the drug by engineering the article or implant to provide constant release over a comparatively shorter period of time. In some embodiments, the drug release is tailored for dose and duration suitable for the indication or administration method.

In some embodiments, the release rate is related to, for example, the drug configuration of the dimer. In some embodiments, the drug release rate from an article or implant described herein is modulated by the cleavage of dimer-linker bond through hydrolysis or enzymatic degradation. In some embodiments, the linking moiety (e.g., the linker) affects drug release rate. In some embodiments, the drug release rate is controlled by a functional group on the composition described herein to conjugate through to the linker, for example, a primary vs. a secondary hydroxyl group. In some embodiments, the release rate from a dimer is related to percentage of the loaded dimer compared to the final drug dimer formulation (e.g., by using a pharmaceutical excipient (e.g., bulking agent/excipient). In some embodiments, the release rate is controlled by the size of a microbead. In some embodiments, drug release is tailored based on the solubility of drug dimer (e.g., through selection of appropriate drug and/or linker) that will influence the rate of surface erosion (e.g., dissolution/degradation) from the article or implant. In other embodiments, drug release is affected by changes in surface area of the formulation, e.g., by changing the diameter of the microbeads. By adjusting the vide supra factors, dissolution, degradation, diffusion, and controlled release may be varied over wide ranges. For example, release may be designed to be initiated over minutes to hours, and may extend over the course of days, weeks, months, or years.

In some embodiments, an implant, article, or composition described herein is suitable for ophthalmic administration. In some embodiments, the ophthalmic administration is intraocular, subretinal, superciliary, forniceal, into Schlemm's canal, inside a bleb, intracameral, intravitreal, suprachoroidal, punctal, retrobulbar, or subconjunctival.

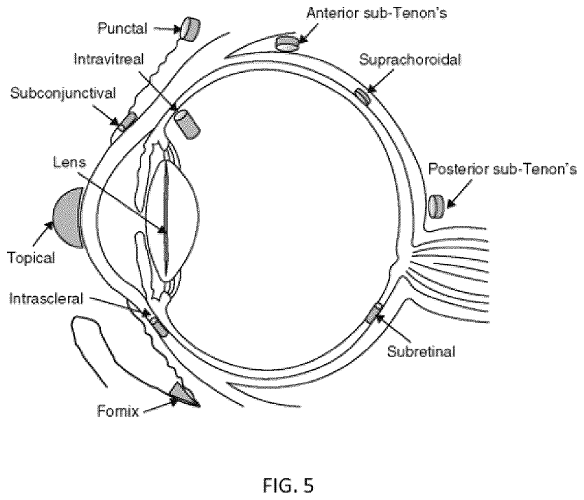

In some embodiments, an implant, article, or composition described herein is suitable for intravitreal administration, suprachoroidal administration, subretinal administration, anterior sub-tenon's administration, posterior sub-tenon's administration, posterior juxtascleral administration, anterior juxtascleral administration, subconjunctival administration, topical administration, intrascleral administration, or punctal administration. In some embodiments, an implant, article, or composition described herein is suitable for intravitreal administration. In some embodiments, an implant, article, or composition described herein is suitable for suprachoroidal administration. FIG. 5 illustrates exemplary insertion locations for various rotes of administration identified herein.

The dose of the composition comprising at least one compound as described herein differ, depending upon the individual's (e.g., human) condition, that is, general health status, age, and other factors.

Pharmaceutical compositions are administered in a manner appropriate to the disease to be treated (or prevented). An appropriate dose and a suitable duration and frequency of administration will be determined by such factors as the condition of the individual, the type and severity of the individual's disease, the particular form and/or potency of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity).

Optimal doses are generally determined using experimental models and/or clinical trials. The optimal dose depends upon the body mass, weight, or blood volume of the individual.

In other embodiments, the compositions described herein are combined with a pharmaceutically suitable or acceptable carrier (e.g., a pharmaceutically suitable (or acceptable) excipient, physiologically suitable (or acceptable) excipient, or physiologically suitable (or acceptable) carrier. Exemplary excipients are described, for example, in Remington: The Science and Practice of Pharmacy (Gennaro, 21^{st} Ed. Mack Pub. Co., Easton, PA (2005)).

Provided in some embodiments herein is a method of administering an implant described herein to an individual. In some embodiments, the method comprises inserting the implant into the individual. In some embodiments, the method comprises inserting the implant into an eye of the individual.

Provided in some embodiments herein is a method of treating a medical indication or abnormality (e.g., an ophthalmic disease and/or disorder), the method comprising administering a therapeutically effective amount of a compound or composition provided herein.

In some embodiments, the medical indication or abnormality is an inflammatory indication or abnormality.

In some embodiments, the medical indication or abnormality is an indication that is treated, prevented, managed, or the like with a drug described in Table 2.

In some embodiments, the medical indication or abnormality is an indication that is treated, prevented, managed, or the like with a therapeutic agent of a drug class described in Table 2.

In some embodiments, the medical indication or abnormality is an indication that is treated, prevented, managed, or the like with a therapeutic agent provided in Table 2.

In some embodiments, the medical indication or abnormality is an indication that is treated, prevented, managed, or the like with rosuvastatin.

In some embodiments, a composition provided herein (e.g., used in a method provided herein) comprises a compound provided herein in a therapeutically effective amount (e.g., at a concentration effective to treat an ophthalmic disease or disorder in an individual in need thereof, the method comprising administering to the individual a compound, pharmaceutically acceptable salt, implant, article, or composition having the structure of any one of Formula 1, Formula I, Formula (ID), Formula IC, Formula IC′, Formula IC1, Formula IC1′, Formula D, Formula D′, Formula IA, Formula IA′, Formula IB, or Formula IB′. In some embodiments, a composition provided herein (e.g., used in a method provided herein) comprises a compound provided herein in a therapeutically effective amount (e.g., at a concentration effective to age-related macular degeneration (AMD)) in the eye.

In some embodiments, provided herein is a method of treating an ophthalmic disease, disorder, or condition in an individual (e.g., in need of thereof), comprising administering to the individual a (e.g., pharmaceutical) composition provided herein, such as, comprising any article or implant containing any compound provided herein, or a pharmaceutically acceptable salt thereof, such as at least one therapeutic agent and a compound having a structure represented by any one of Formula 1, Formula I, Formula (ID), Formula IC, Formula IC′, Formula IC1, Formula IC1′, Formula D, Formula D′, Formula IA, Formula IA′, Formula IB, or Formula IB′.

In some embodiments, a composition provided herein (e.g., used in a method provided herein) comprises a compound provided herein in a therapeutically effective amount (e.g., at a concentration effective to treat an ophthalmic disease or disorder in an individual in need thereof, the method comprising administering to the individual a compound, pharmaceutically acceptable salt, implant, article, or composition having the structure of any one of Formula II, Formula ID, Formula IIC, Formula IIC′, Formula IIA, Formula IIA′, Formula IIB, Formula IIB′, or a compound provided in Table 4. In some embodiments, a composition provided herein (e.g., used in a method provided herein) comprises a compound provided herein in a therapeutically effective amount (e.g., at a concentration effective to age-related macular degeneration (AMD)) in the eye.

In some embodiments, provided herein is a method of treating an ophthalmic disease, disorder, or condition in an individual (e.g., in need of thereof), comprising administering to the individual a (e.g., pharmaceutical) composition provided herein, such as, comprising any article or implant containing any compound provided herein, or a pharmaceutically acceptable salt thereof, such as at least one therapeutic agent and a compound having a structure represented by any one of Formula II, Formula ID, Formula IIC, Formula IIC′, Formula IIA, Formula IIA′, Formula IIB, Formula IIB′, or a compound provided in Table 4.

Provided in some embodiments herein is a method of modulating lipids in an eye of an individual, the method comprising administering to the eye of the individual a compound, pharmaceutically acceptable salt, implant, article, or composition of any embodiments provided herein. In some embodiments, modulating lipids comprises reducing lipoprotein levels, reducing lipoprotein production, reducing drusen levels, reducing drusen formation, reducing cholesterol levels, reducing cholesterol synthesis, reducing cholesterol intermediates, reducing oxysterol synthesis, reducing oxysterol formation, reducing oxysterol levels, reducing lipid peroxidation, or a combination thereof (in the eye of the individual).

Provided in some embodiments herein is a method of reducing inflammation in an eye of an individual, the method comprising administering to the eye of the individual a compound, pharmaceutically acceptable salt, implant, article, or composition of any embodiments provided herein. In some embodiments, reducing inflammation comprises decreasing oxidative stress, or pro-inflammatory factors in the eye of the individual.

Provided in some embodiments herein is a method of reducing oxidative stress in an eye of an individual, the method comprising administering to the eye of the individual any compound, pharmaceutically acceptable salt, implant, article, or composition described herein. In some embodiments, a reduction in oxidative stress is determined by a change (e.g., decrease) in lipid peroxidation and/or pro-inflammatory markers.

Provided in some embodiments herein is a method of reducing lipid peroxidation in an eye of an individual, the method comprising administering to the eye of the individual any compound, pharmaceutically acceptable salt, implant, article, or composition described herein.

Provided in some embodiments herein is a method of reducing lipoprotein in an eye of an individual, the method comprising administering to the eye of the individual any compound, pharmaceutically acceptable salt, implant, article, or composition described herein. In some embodiments, the lipoprotein comprises a cholesterol or a cholesterol ester.

Provided in some embodiments herein is a method of reducing lipoprotein formation in an eye of an individual, the method comprising administering to the eye of the individual any compound, pharmaceutically acceptable salt, implant, article, or composition described herein. In some embodiments, the lipoprotein comprises a cholesterol or a cholesterol ester.

Provided in some embodiments herein is a method of reducing oxysterol in an eye of an individual, the method comprising administering to the eye of the individual any compound, pharmaceutically acceptable salt, implant, article, or composition described herein. In some embodiments, reducing oxysterol comprises reducing the rate of oxysterol formation. In some embodiments, reducing cholesterol comprises reducing the rate of oxysterol synthesis.

Provided in some embodiments herein is a method of reducing oxysterol synthesis in an eye of an individual, the method comprising administering to the eye of the individual any compound, pharmaceutically acceptable salt, implant, article, or composition described herein.

Provided in some embodiments herein is a method of reducing cholesterol in an eye of an individual, the method comprising administering to the eye of the individual any compound, pharmaceutically acceptable salt, implant, article, or composition described herein. In some embodiments, reducing cholesterol comprises reducing the rate of cholesterol formation. In some embodiments, reducing cholesterol comprises reducing the rate of cholesterol synthesis. In some embodiments, reducing cholesterol comprises reducing cholesterol intermediates. In some embodiments, reducing cholesterol comprises reducing rate of formation or synthesis of cholesterol intermediates.

Provided in some embodiments herein is a method of reducing cholesterol synthesis in an eye of an individual, the method comprising administering to the eye of the individual any compound, pharmaceutically acceptable salt, implant, article, or composition described herein.

Provided in some embodiments herein is a method of reducing drusen in an eye of an individual, the method comprising administering to the eye of the individual any compound, pharmaceutically acceptable salt, implant, article, or composition described herein. In some embodiments, drusen comprises lipoprotein, such as a lipoprotein comprising a cholesterol or a cholesterol ester.

Provided in some embodiments herein is a method of reducing drusen formation in an eye of an individual, the method comprising administering to the eye of the individual any compound, pharmaceutically acceptable salt, implant, article, or composition described herein. In some embodiments, reducing drusen formation comprises reducing the rate of drusen formation. In some embodiments, drusen comprises lipoprotein, such as a lipoprotein comprising a cholesterol or a cholesterol ester.

Provided in some embodiments herein is a method of inhibiting 3-hydroxy-3-methylglutaryl coenzyme A (HMG-CoA) in an eye of an individual, the method comprising administering to the eye of the individual any compound, pharmaceutically acceptable salt, implant, article, or composition described herein.

Provided in some embodiments herein is a method of reducing apoptosis (e.g., in an eye of) an individual, the method comprising administering to the eye of the individual a compound, pharmaceutically acceptable salt, implant, article, or composition of any embodiments provided herein.

Provided in some embodiments herein is a method of modulating complement-mediated damage (e.g., in an eye of) an individual, the method comprising administering to the eye of the individual a compound, pharmaceutically acceptable salt, implant, article, or composition of any embodiments provided herein.

Provided in some embodiments herein is a method of reducing endothelial dysfunction (e.g., in an eye of) an individual, the method comprising administering to the eye of the individual a compound, pharmaceutically acceptable salt, implant, article, or composition of any embodiments provided herein.

Provided in some embodiments herein is a method of reducing vascular remodeling (e.g., in an eye of) an individual, the method comprising administering to the eye of the individual a compound, pharmaceutically acceptable salt, implant, article, or composition of any embodiments provided herein.

Provided in some embodiments herein is a method of reducing extracellular remodeling (e.g., in an eye of) an individual, the method comprising administering to the eye of the individual a compound, pharmaceutically acceptable salt, implant, article, or composition of any embodiments provided herein.

Provided in some embodiments herein is a method of providing immunomodulatory effects (e.g., in an eye of) an individual, the method comprising administering to the eye of the individual a compound, pharmaceutically acceptable salt, implant, article, or composition of any embodiments provided herein.

Provided in some embodiments herein is a method of providing anti-oxidant effects (e.g., in an eye of) an individual, the method comprising administering to the eye of the individual a compound, pharmaceutically acceptable salt, implant, article, or composition of any embodiments provided herein.

Provided in some embodiments herein is a method of providing neuroprotective effects in an individual, the method comprising administering to the eye of the individual a compound, pharmaceutically acceptable salt, implant, article, or composition of any embodiments provided herein.

Provided in some embodiments herein is a method of increasing vascular perfusion in an eye of an individual, the method comprising administering to the eye of the individual any compound, pharmaceutically acceptable salt, implant, article, or composition described herein.

In some embodiment, any pharmaceutical composition, implant, or article provided herein or used in a method provided herein is in the form of a solid suitable for intraocular administration (e.g., injection or insertion). In some embodiments, intraocular administration is intraocular, subretinal, superciliary, forniceal, into Schlemm's canal, inside a bleb, intracameral, intravitreal, suprachoroidal, punctal, retrobulbar, or subconjunctival.

Methods involving treating an individual, in some instances, include preventing a disease, disorder or condition from occurring in the subject which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected (e.g., such as decreasing the ocular condition of an individual by administration of an agent even though such agent does not treat the cause of the ocular condition).

In some instances, provided herein is a method of treating one or more ophthalmic disease, disorder, or condition in an individual (e.g., in need of thereof).

In some embodiments, the ocular disease or disorder is a disease or disorder that causes (e.g., progressive) vison loss.

In some embodiments, the ocular disease or disorder is a disease or disorder of a macula, cornea, retina, choroid, eyelid, orbit, conjunctiva, vitreous, lens, optic nerve, or any combination thereof.

In some embodiments, the ocular disease or disorder is macular degeneration (e.g., age-related macular degeneration (AMD), such as wet AMD or dry AMD), dry eye, blepharitis, graft-versus-host disease, corneal wound healing, thyroid-associated ophthalmopathy, orbital myositis, ocular inflammatory disease (e.g., uveitis), cataracts, open angle glaucoma, vitreous and preretinal hemorrhage, proliferative vitreoretinal disease, diabetic retinopathy, retinal vein occlusion, choroideremia, choroidal vessel occlusive disease, or choroidal melanoma.

In some embodiments, the ocular disease or disorder is macular degeneration (e.g., age-related macular degeneration (AMD), such as dry AMD). In some embodiments, the ocular disease or disorder is intermediate AMD. In some embodiments, the ocular disease or disorder is geographic atrophy.

In some instances, an implant described herein releases into a local environment described herein in a controlled manner, such as having a zero order or near zero order release profile (e.g., at least while the surface area of the implant remains substantially similar to the original, implanted surface area of the implant). In some instances, such release facilitates treatment of a localized disease (e.g., ocular disease, such as described herein) or treatment of local effects or symptoms of a disease described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1: Analytical Methods

### Analytical Example 1: High Performance Liquid Chromatography (HPLC):

Samples (20.0 mg) are dissolved in acetonitrile (10.0 mL) to make 2 mg/mL solution. For the system: solvent A was Water+0.05% trifluoroacetic acid (TFA); solvent B was Acetonitrile+0.05% TFA; the flow rate was 1.0 mL/min; and the detection method was UV @242 nm and UV Spectra from 190 to 400 nm (Reference Wavelength Off). The samples were loaded onto an Agilent 1100 series HPLC with a Phenomenex Gemini-NX C18 Column (5 μm; 110 Å; 250×4.6 mm; 00G-4454-E0) and Phenomenex SecurityGuard Analytical Guard Column (KJO-4282) with Gemini C18 4×3.0 mm Guard Cartridge (AJO-7597) connected in-series. Column temperature was held at 25C. The solvent gradient profile is shown in Table 5.

TABLE 5

| Time (min) | % A Solvent | % B Solvent |
|---|---|---|
| 0 | 80 | 20 |
| 40 | 16 | 84 |
| 42 | 0 | 100 |
| 50 | 0 | 100 |

### Analytical Example 2: Nuclear Magnetic Resonance (NMR)

Compounds (10 mg) were dissolved in 666 μL of either CDCl3 or DMSO-d6 and loaded in an 8-inch length, 5 mm diameter NMR tube. The instrument was a Varian Mercury 400 NMR spectrometer. Proton NMR spectra were obtained with 16 scans using the default method. FIDs were processed with MestRe-C software.

### Analytical Example 3: Mass Spectrometry (MS)

Compounds were dissolved in acetonitrile at 1 mg/ml and used directly for analysis on an Agilent 6538 QTOF, using ESI MS+ as ion source.

### Analytical Example 4: Melting Point

Compound powder was prepared neat in a glass capillary tube, and melting temperature was measured manually with standard glass capillary tube melting point apparatus.

### Analytical Example 5: Differential Scanning Calorimetry (DSC)

5-10 mg of compounds were weighed in an aluminum pan. Using a Hitachi Differential Scanning calorimeter DSC7020, samples were heated from room temperature to 150-200° C. at 10° C./min, cooled to −30° C. at 10° C./min, and heated again to 150-200° C. at 10° C./min.

### Example 2: Chemical Synthesis

Solvents, reagents and starting materials were purchased from commercial vendors and used as received unless otherwise described. All reactions were performed at room temperature unless otherwise stated. Starting materials were purchased from commercial sources or synthesized according to the methods described herein or using literature procedures.

### Chemical Synthesis Example 1

### 2-((8S,10S,13S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethyl (3R,5S,E)-7-(4-(4-fluorophenyl)-6-isopropyl-2-(N-methylmethylsulfonamido)pyrimidin-5-yl)-3,5-dihydroxyhept-6-enoate (Compound 1)

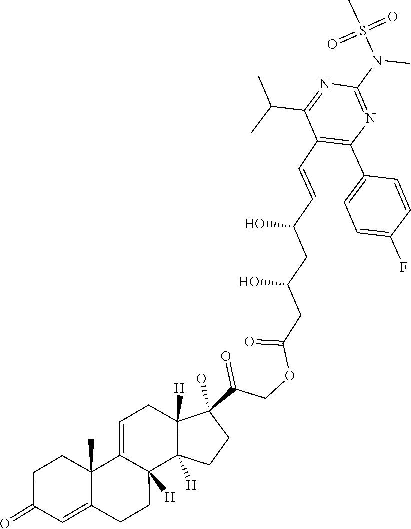

To a stirred solution of rosuvastatin sodium (200 mg, 0.397 mmol) in DMF (2 mL) was added anecortave mesylate (168 mg, 0.397 mmol) and the mixture stirred for 16 h at 37° C. The mixture was concentrated onto 1 g reverse phase silica and purified by reverse phase column chromatography using a 20-100% Water-MeCN gradient. Product containing fractions were combined and concentrated to give the product (184 mg, 57%) as an off-white solid. HPLC retention time: 32.5 min. ESI MS+ calculated for C₄₃H₅₄FN₃O₉S⁺; 808.3638, Found: 808.3626 1H NMR (400 MHz, DMSO) δ 7.80-7.66 (m, 2H), 7.36-7.18 (m, 2H), 6.50 (d, J=16.2 Hz, 1H), 5.65 (s, 1H), 5.62-5.42 (m, 3H), 5.06-4.80 (m, 3H), 4.75 (d, J=5.4 Hz, 1H), 4.29-4.14 (m, 1H), 3.88 (d, J=5.6 Hz, 1H), 3.54 (s, 3H), 3.44 (s, 4H), 2.75-2.39 (m, 6H), 2.39-2.11 (m, 3H), 2.12-1.91 (m, 3H), 1.81 (d, J=12.9 Hz, 2H), 1.57 (tt, J=11.8, 7.4 Hz, 3H), 1.29 (s, 4H), 1.21 (dd, J=6.7, 2.6 Hz, 6H), 0.99 (q, J=12.7 Hz, 1H), 0.44 (s, 3H). Tm: 120° C.

### Chemical Synthesis Example 2

### 2-((8S,10S,13S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethyl 2-chloroacetate (Anecortave Acetyl Chloride)

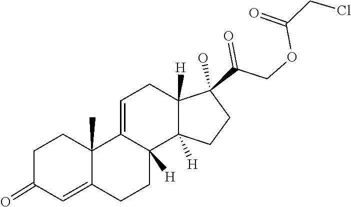

To a stirred solution of anecortave des acetate (344 mg, 1.0 mmol), triethylamine (279 uL, 2.0 mmol) and DMAP (244 mg, 2.0 mmol) in dry THE (50 mL) was added chloroacetyl chloride (320 uL, 4.0 mmol) dropwise and the mixture stirred at RT for 16 h. The mixture was concentrated onto 1 g reverse phase silica and purified by reverse phase column chromatography using a 20-100% Water-MeCN gradient. Product containing fractions were combined and concentrated to give the product (232 mg, 39%) as an off-white solid. HPLC retention time: 13.8 min. ESI MS+ calculated for C₂₃H₃₀ClO₅⁺; 421.1776, Found: 421.1783; 1H NMR (400 MHz, DMSO) δ 5.66 (s, 1H), 5.57 (s, 1H), 5.54 (dt, J=6.2, 1.8 Hz, 1H), 5.14 (d, J=17.5 Hz, 1H), 4.94 (d, J=17.5 Hz, 1H), 4.51 (s, 2H), 2.69-2.50 (m, 3H), 2.50-2.43 (m, 1H), 2.37-2.21 (m, 3H), 2.14-2.05 (m, 1H), 2.02-1.93 (m, 2H), 1.88-1.77 (m, 2H), 1.76-1.67 (m, 1H), 1.65-1.53 (m, 1H), 1.43-1.30 (m, 1H), 1.20 (s, 3H), 1.09-0.93 (m, 1H), 0.90 (s, 3H).

### Chemical Synthesis Example 3

### 2-(2-((8S,10S,13S,14S,17R)-17-hydroxy-10,13-dimethyl-3-oxo-2,3,6,7,8,10,12,13,14,15,16,17-dodecahydro-1H-cyclopenta[a]phenanthren-17-yl)-2-oxoethoxy)-2-oxoethyl (3R,5S,E)-7-(4-(4-fluorophenyl)-6-isopropyl-2-(N-methylmethylsulfonamido)pyrimidin-5-yl)-3,5-dihydroxyhept-6-enoate (Compound 2)

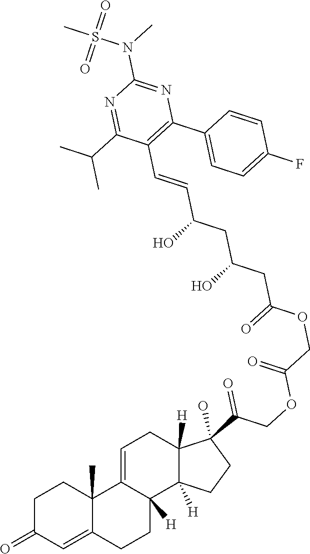

To a stirred solution of rosuvastatin sodium (333 mg, 0.69 mmol) in DMF (15 mL) was added anecortave acetyl chloride (291 mg, 0.69 mmol) and sodium iodide (207 mg, 1.38 mmol). The mixture was stirred at 37° C. for 3d. The mixture was concentrated onto 1 g reverse phase silica and purified by reverse phase column chromatography using a 20-100% water-MeCN gradient. Product containing fractions were combined and concentrated to give the product (291 mg, 69%) as an off-white solid. HPLC retention time: 13.8 min; ESI MS+ calculated for C₄₅H₅₇FN₃O₁₁S⁺; 866.3692, Found: 866.3708; 1H NMR (400 MHz, DMSO) δ 7.76-7.65 (m, 2H), 7.29 (dt, J=12.9, 8.9 Hz, 2H), 6.52 (dd, J=16.1, 1.3 Hz, 1H), 5.65 (s, 1H), 5.60-5.46 (m, 3H), 5.08 (t, J=17.5 Hz, 1H), 4.97-4.84 (m, 2H), 4.84-4.70 (m, 2H), 4.21 (s, 1H), 3.55 (d, J=3.1 Hz, 3H), 3.48-3.38 (m, 5H), 2.70-2.51 (m, 4H), 2.42 (dd, J=15.1, 8.0 Hz, 2H), 2.32 (d, J=14.5 Hz, 1H), 2.23 (s, 1H), 2.07 (s, 5H), 1.88-1.72 (m, 3H), 1.56 (dd, J=14.0, 6.8 Hz, 2H), 1.45 (dt, J=13.2, 6.0 Hz, 1H), 1.36-1.18 (m, 10H), 1.00 (d, J=13.5 Hz, 1H), 0.48 (d, J=13.7 Hz, 3H). Tm=128° C.

### Example 3: Formation and Evaluation of Processable Compounds

### Process Example 1: Heat Processing Pellets

A compound provided herein is formed into a pellet in the glassy state by heat molding. Crystalline powder of the compound is melted at a temperature of 155° C. to 225° C. and pressed into a cylindrical mold of ~1 mm height×1 mm diameter.

### Process Example 2: Heat Processing Rods

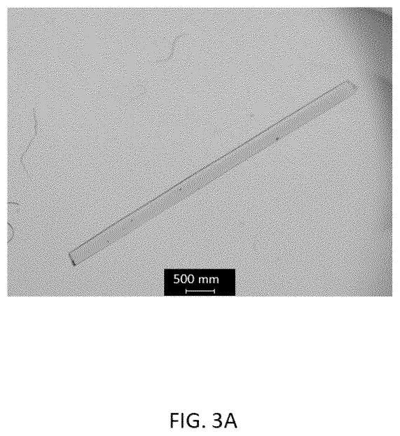

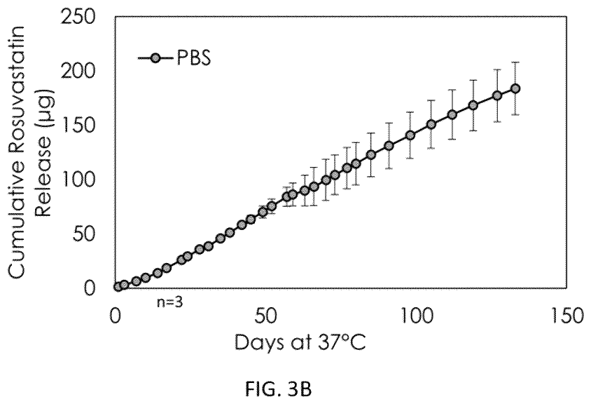

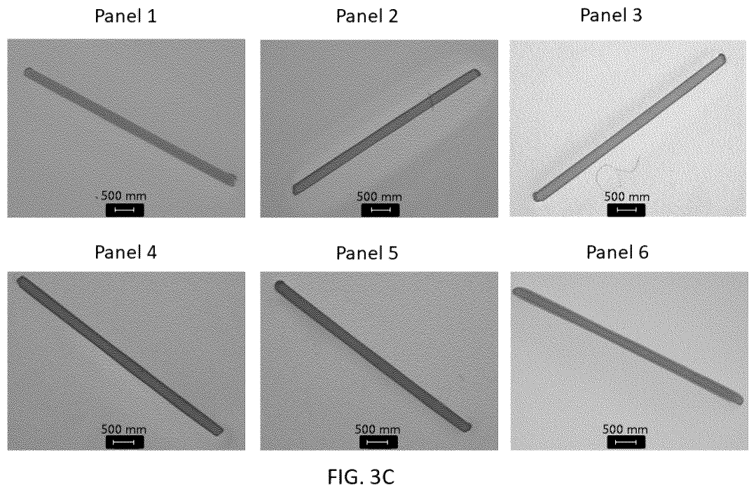

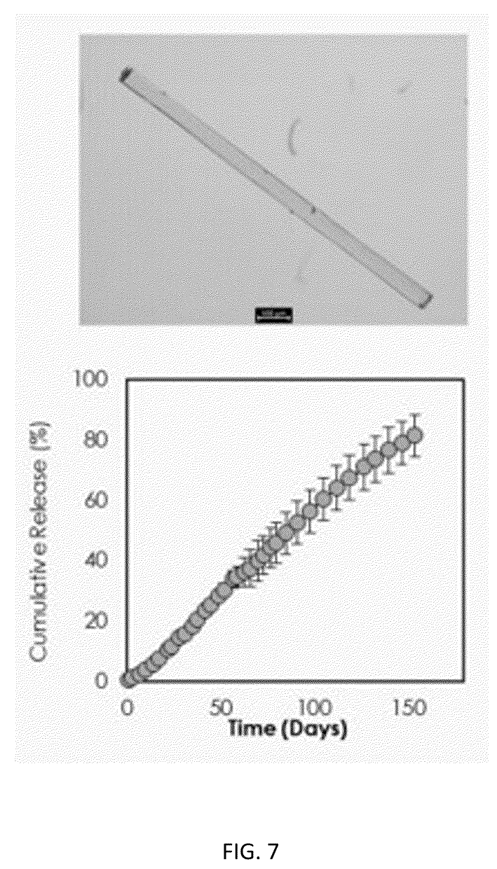

A compound of the disclosure was formed into a rod in the glassy state by heat extrusion. The conjugate compound was initially melted at a temperature up to 160° C. The resulting material was then loaded into a heat extruder with a 26G die head, heated at 95° C. to 115° C., and pressure was applied to a piston to form extrudate from the extruder. The extrudate was cut to different lengths (e.g., see FIG. 3A, FIG. 3C, and FIG. 7).

### Example 4: Drug Release Evaluation from Pellets or Extruded Rods

Drug release from heat-molded pellets or extruded rods of Compounds of the disclosure were assessed in either fetal bovine serum (FBS) or phosphate buffered saline (PBS). Heat-molded pellets or extruded rods were placed in 20 ml glass vials, to which is added 2 mL of release buffer. Samples are incubated at 37° C. with constant agitation at 115 rpm. At intervals up to 14 days in length, release buffer was assessed for released drug and then fully replaced with 2 mL of fresh buffer. For FBS release conditions, acetonitrile was added to precipitate proteins and extract drug release products. Samples were analyzed by high performance liquid chromatography (HPLC) to quantify drug products (e.g., see FIG. 3B and FIG. 7).

### Example 5: In Vivo Implantation and Evaluation

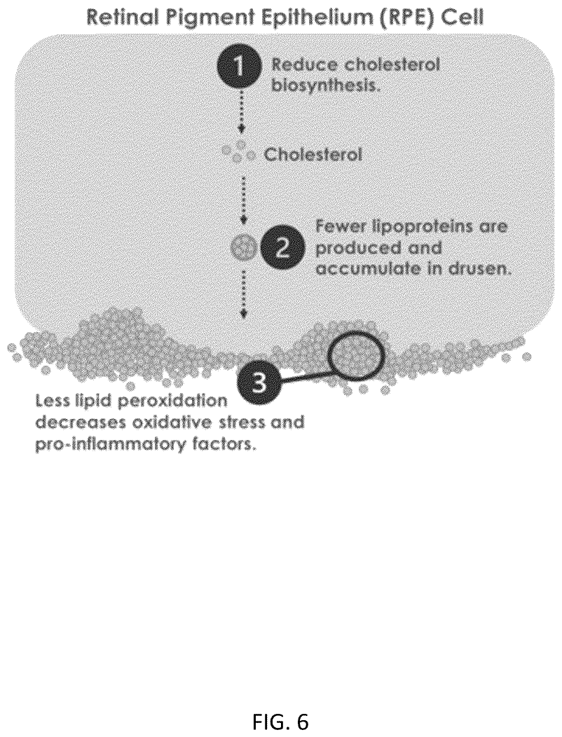

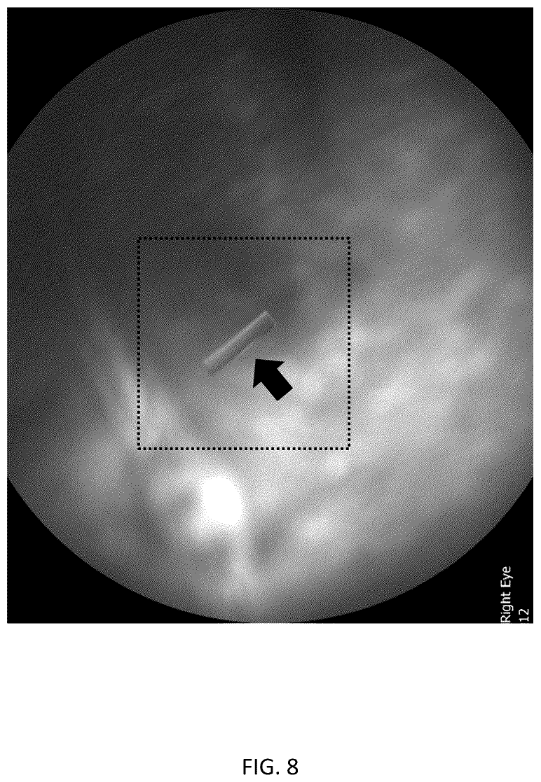

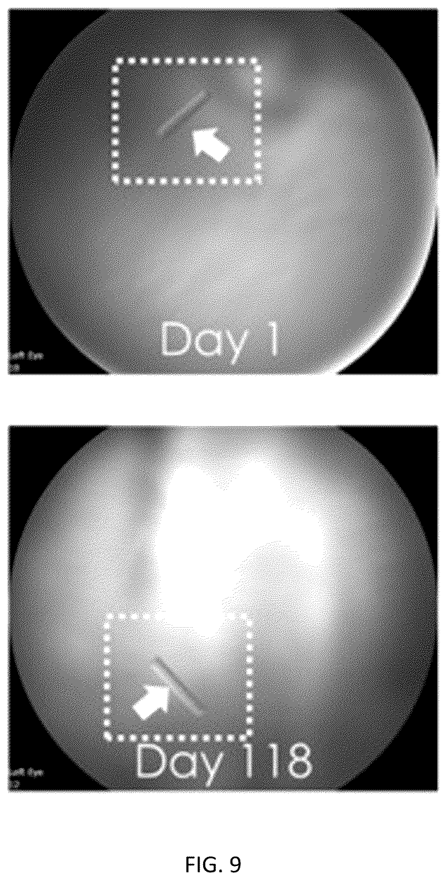

Compounds described herein are formed into rods (e.g., according to the methods described herein) and cut to have a certain length (e.g., 1- or 6-mm length implants). The implants are loaded in the lumen of needles, terminally sterilized, and injected into the vitreous of rabbits. Implants remain suspended in the vitreous and were visualized, such as by fundus imaging (e.g., see FIG. 8 and FIG. 9). Implants were tolerated in the eye for months (e.g., see FIG. 6).

### Example 6: Intravitreal Statin Impact on Cholesterol Biosynthesis Pathway

An extruded rod provided herein was cut to provide an intravitreal implant. The resulting implant was loaded in the lumen of needles and terminally sterilized. The implant was administered intravitreally to rabbits in one eye, with the contralateral eye receiving a sham intravitreal procedure. One-month post-implantation, sham and implant treated eyes were collected and retinal tissue was assessed for levels of different components of the cholesterol biosynthesis pathway, such as cholesterol, desmosterol, lanosterol, lathosterol, 7 dehydroxycholesterol, 8 dehydroxycholesterol, and 24-hydroxycholesterol.

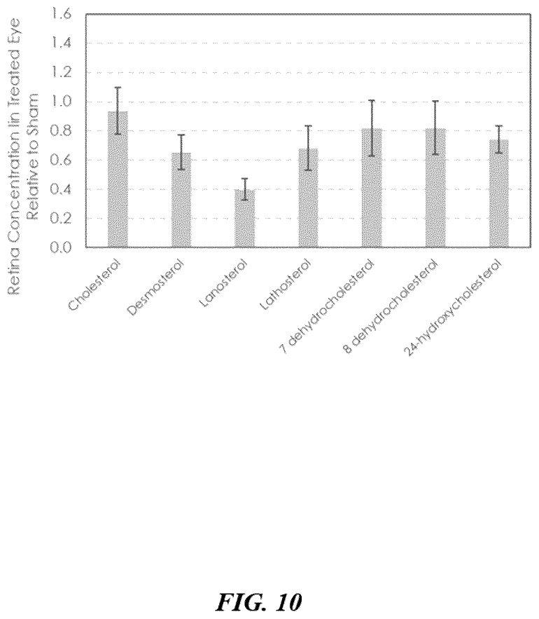

As illustrated in FIG. 10, the components of the cholesterol biosynthesis pathway evaluated in the collected eye samples were reduced compared to sham eyes when an extruded rod comprising Compound 1 was implanted into sham eyes. As such, FIG. 10 demonstrates that implants described herein can modulate the cholesterol biosynthesis pathway compared to sham eyes, such as to provide a decrease in components of the cholesterol biosynthesis pathway found in the eye. A decrease in levels of components of the cholesterol biosynthesis pathway (e.g., in diseased eyes), especially over a prolonged period of time with the use of implants described herein, may be advantageous for certain individuals who have elevated levels of components of the cholesterol biosynthesis pathway in one or more of their eyes.

We claim:

1. A compound having a structure represented by Formula IIA: 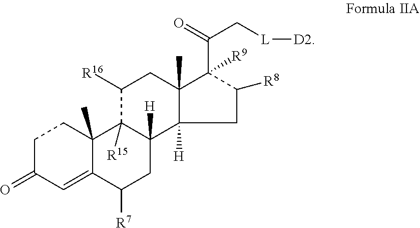
wherein: [symbol]
is a single bond or a double bond;
R⁷ is hydrogen or halogen;
R⁸ is hydrogen or C₁-C₄ alkyl;
R⁹ is absent, hydrogen, or hydroxyl;
R¹⁵ is absent, hydrogen, or halogen;
R¹⁶ is hydrogen or hydroxyl;
D2 is a radical of rosuvastatin; and
L is a linker,
or a pharmaceutically acceptable salt or solvate thereof.

2. The compound of claim 1, wherein R⁷ is hydrogen.

3. The compound of claim 1, wherein R⁸ is hydrogen.

4. The compound of claim 1, wherein R⁹ is hydroxyl.

5. The compound of claim 1, wherein R¹⁵ is absent.

6. The compound of claim 1, wherein R¹⁶ is hydrogen.

7. The compound of claim 1, wherein the compound has a structure represented by Formula IIB:

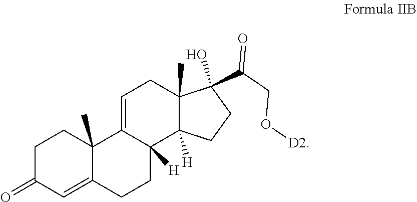

8. The compound of claim 1, wherein D2 has a structure represented by Formula A:

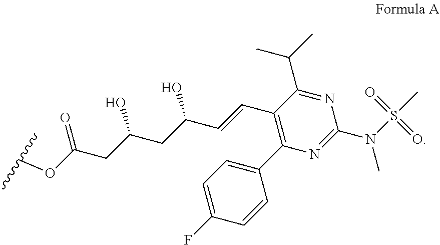 [SMILES: *OC(=O)C[C@H](O)C[C@H](O)/C=C/c1c(-c2ccc(F)cc2)nc(N(C)S(C)(=O)=O)nc1C(C)C]

9. The compound of claim 7, wherein D2 has a structure represented by Formula A:

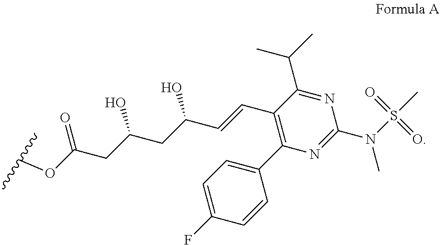 [SMILES: *OC(=O)C[C@H](O)C[C@H](O)/C=C/c1c(-c2ccc(F)cc2)nc(N(C)S(C)(=O)=O)nc1C(C)C]

10. The compound of claim 1, wherein the linker is a bond.

11. The compound of claim 1, wherein the linker is or comprises substituted or unsubstituted alkyl.

12. The compound of claim 11, wherein the linker is substituted alkyl.

13. The compound of claim 12, wherein the linker is or comprises alkyl substituted with oxo.

14. The compound of claim 13, wherein the linker is or comprises —(O═C)CH₂—.

15. A compound having the structure: 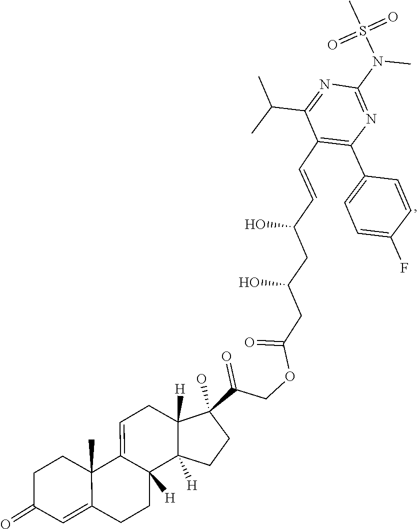
or a pharmaceutically acceptable salt thereof.

16. A compound having the structure: 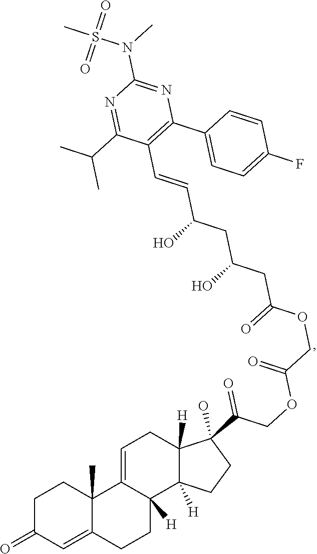
or a pharmaceutically acceptable salt thereof.

17. A pharmaceutical implant comprising a compound of the compound of claim 1, or a pharmaceutically-acceptable salt thereof.

18. The pharmaceutical implant of claim 17, wherein the implant comprises at least 70 wt. % of the compound or pharmaceutically acceptable salt thereof.

19. The pharmaceutical implant of claim 17, wherein the implant comprises at least 90 wt. % of the compound or pharmaceutically acceptable salt thereof.

20. A method of modulating lipids in an eye of an individual, the method comprising administering to the eye of the individual a compound, pharmaceutically acceptable salt, of claim 1.
